(19) United States
(12) Patent Application Publication
(10) Pub. No.: US 2021/0119135 A1
(43) Pub. Date: Apr. 22, 2021

(54) LUMINESCENCE DEVICE AND AMINE COMPOUND FOR LUMINESCENCE DEVICE

(71) Applicant: Samsung Display Co., LTD., Yongin-si (KR)

(72) Inventor: Takuya UNO, Yokohama (JP)

(21) Appl. No.: 17/030,269

(22) Filed: Sep. 23, 2020

(30) Foreign Application Priority Data
Oct. 21, 2019 (KR) 10-2019-0130480

(51) Int. Cl. H01L 51/00; C07D 333/76; C07D 409/12; C07D 307/91

(52) U.S. Cl. CPC H01L 51/0061; C07D 333/76; H01L 51/006; H01L 51/5012; C07D 307/91; H01L 51/0074; H01L 51/0073; C07D 409/12

(57) ABSTRACT

A luminescence device, includes: a first electrode; a hole transport region disposed on the first electrode; an emission layer disposed on the hole transport region; an electron transport region disposed on the emission layer; and a second electrode disposed on the electron transport region, wherein the hole transport region includes an amine compound of Formula 1, as defined herein.

### CROSS REFERENCE TO RELATED APPLICATIONS

This application claims priority from and the benefit of Korean Patent Application No. 10-2019-0130480, filed on Oct. 21, 2019, which is hereby incorporated by reference for all purposes as if fully set forth herein.

### BACKGROUND

### Field

Exemplary embodiments of the invention relate generally to a luminescence device and, and more particularly, a luminescence device including an amine compound.

### Discussion of the Background

As an image display, the development of an electroluminescence display is being actively conducted. As an example, the electroluminescence display may include an organic electroluminescence display. The organic electroluminescence display is different from a liquid crystal display and is a so-called self-luminescent display achieved by subjecting for a light-emitting material to emit light in an emission layer.

For a display having a luminescence device, an increase in the efficiency and life of the luminescence device is desirable, and there is continued interest in the development of materials for a luminescence device to stably accomplish these objectives.

The above information disclosed in this Background section is only for understanding of the background of the inventive concepts, and, therefore, it may contain information that does not constitute prior art.

### SUMMARY

Applicant discovered that by combining a hole transport region having an amine compound with one or more other regions and/or layers with different attributes unexpected synergistic improvements in the lifespan and efficiency of luminescence devices can be obtained.

Luminescence devices constructed according to the principles and exemplary implementations of the invention have high efficiency and long life. For example, the hole transport region having the amine compound may optionally be combined with an emission layer having a polycyclic compound to obtain improved high efficiency and long life in luminescence devices.

Additional features of the inventive concepts will be set forth in the description which follows, and in part will be apparent from the description, or may be learned by practice of the inventive concepts.

According to one aspect of the invention, a luminescence device includes:

a first electrode;

a hole transport region disposed on the first electrode;

an emission layer disposed on the hole transport region;

an electron transport region disposed on the emission layer; and

a second electrode disposed on the electron transport region,

wherein the hole transport region includes an amine compound of Formula 1:

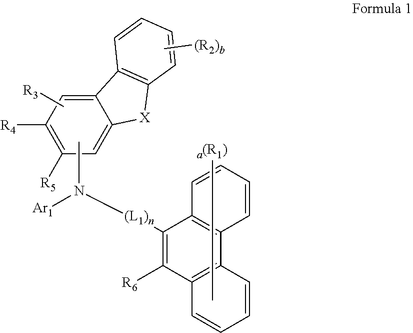

in Formula 1,

X is O or S;

R₁ to R₅ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;

R₆ is a hydrogen atom, or a deuterium atom;

L₁ is a substituted or unsubstituted arylene group of 6 to 40 carbon atoms for forming a ring, or a substituted or unsubstituted heteroarylene group of 2 to 40 carbon atoms for forming a ring;

Ar₁ is a substituted or unsubstituted aryl group of 6 to 40 carbon atoms for forming a ring, or a substituted or unsubstituted heteroaryl group of 2 to 12 carbon atoms for forming a ring;

if X is O, and Ar₁ is a substituted or unsubstituted aryl group with 16 to 40 carbon atoms forming a ring;

a is an integer of 0 to 8;

b is an integer of 0 to 4; and

n is an integer of 1 to 3.

The hole transport region may include a hole injection layer disposed on the first electrode, and a hole transport layer disposed on the hole injection layer, and the hole transport layer may include the amine compound.

The emission layer may include a polycyclic compound of Formula A:

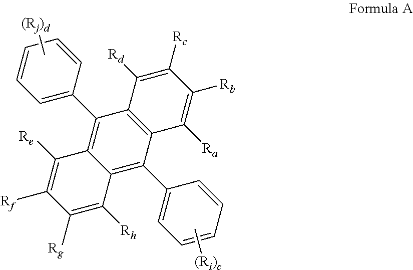

in Formula A, the variables are defined herein.

The variable Ar₁ may be a group of Formula 2, defined herein.

The variable Ar₁ may be a group of Formulae 1-1 to 1-10, defined herein.

The variable Ar₁ may be a group of Formulae 1-11 to 1-20, defined herein.

The variable L₁ may be a group of Formulae 2-1 to 2-4, defined herein.

The variable L₁ may be a group of Formulae 2-11 to 2-14, defined herein.

The amine compound of formula 1 may be a compound of Formula 1-1:

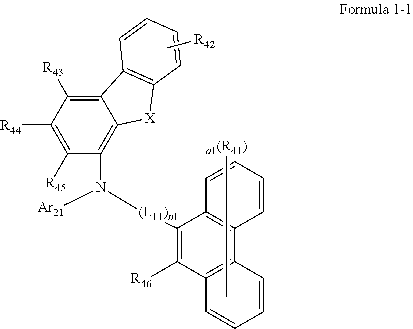

in Formula 1-1, the variables are defined herein.

The amine compound of formula 1 may be a compound represented by the following Formula 1-2:

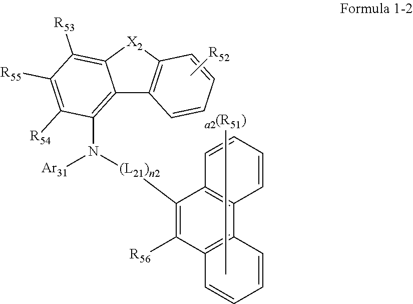

in Formula 1-2, the variables defined herein.

The amine compound of formula 1 may include at least one compound from Compound Groups A-D, as defined herein.

According to another aspect of the invention, an amine compound for use in a luminescence device is of the following Formula 1:

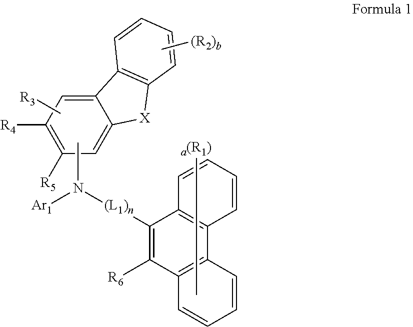

in Formula 1,

X is O or S;

R₁ to R₅ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;

R₆ is a hydrogen atom or a deuterium atom;

L₁ is a substituted or unsubstituted arylene group of 6 to 40 carbon atoms for forming a ring, or a substituted or unsubstituted heteroarylene group of 2 to 40 carbon atoms for forming a ring;

Ar₁ is a substituted or unsubstituted aryl group of 6 to 40 carbon atoms for forming a ring, or a substituted or unsubstituted heteroaryl group of 2 to 12 carbon atoms for forming a ring;

if X is O, and Ar₁ is a substituted or unsubstituted aryl group with 16 to 40 carbon atoms forming a ring;

a is an integer of 0 to 8;

b is an integer of 0 to 4; and

n is an integer of 1 to 3.

The variable Ar₁ may be a group of Formula 2, defined herein.

The variable Ar₁ may be a group of Formulae 1-1 to 1-10, as defined herein.

The variable Ar₁ may be represented by a group of Formulae 1-11 to 1-20, as defined herein.

The variable L₁ may be a group of Formulae 2-1 to 2-4, as defined herein.

The variable L₁ may be a group of Formulae 2-11 to 2-14, as defined herein.

The amine compound of formula 1 may be a compound of the following Formula 1-1:

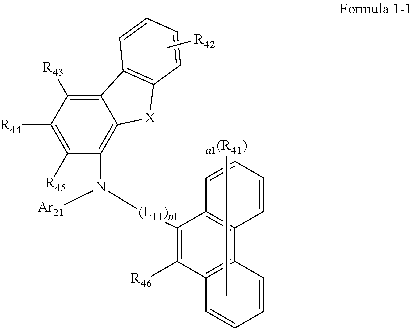

in Formula 1-1, the variables defined herein.

The amine compound of formula 1 may be a compound from the following Formula 1-2:

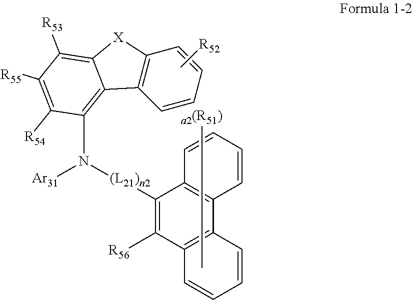

in Formula 1-2, the variables defined herein.

The amine compound of formula 1 may include at least one compound from Compound Groups A-D, as defined herein.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate exemplary embodiments of the invention, and together with the description serve to explain the inventive concepts.

FIG. 1 is a schematic cross-sectional diagram of an exemplary embodiment of a luminescence device constructed according to principles of the invention.

FIG. 2 is a schematic cross-sectional diagram of another exemplary embodiment of a luminescence device constructed according to principles of the invention.

FIG. 3 is a schematic cross-sectional diagram of yet another exemplary embodiment of a luminescence device constructed according to principles of the invention.

FIG. 4 is a schematic cross-sectional diagram of a further exemplary embodiment of a luminescence device constructed according to principles of the invention.

### DETAILED DESCRIPTION

In the following description, for the purposes of explanation, numerous specific details are set forth in order to provide a thorough understanding of various exemplary embodiments or implementations of the invention. As used herein “embodiments” and “implementations” are interchangeable words that are non-limiting examples of devices or methods employing one or more of the inventive concepts disclosed herein. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments. Further, various exemplary embodiments may be different, but do not have to be exclusive. For example, specific shapes, configurations, and characteristics of an exemplary embodiment may be used or implemented in another exemplary embodiment without departing from the inventive concepts.

Unless otherwise specified, the illustrated exemplary embodiments are to be understood as providing exemplary features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an exemplary embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, a film, a region, a plate, is referred to as being “on,” “above,” “below,” “under,” “connected to,” or “coupled to” another element, layer, film, region, or plate, it may be directly on, connected to, or coupled to the other element, layer, film, region, or plate, or intervening elements, layers, films, regions, or plates may be present. When, however, an element, layer, film, region, or plate is referred to as being “directly on,” “directly below,” “directly under,” “directly connected to,” or “directly coupled to” another element or layer, there are no intervening elements, layers, films, regions, or plates present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, numerals, integers, steps, operations, elements, parts, components, and/or groups thereof, but do not preclude the presence or addition of one or more other features, numerals, integers, steps, operations, elements, parts, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

As used herein, the term “substituted” means at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkoxy group, a hydrocarbon ring group, an aryl group, and a heterocyclic group. In addition, each of the substituents may be substituted or unsubstituted. For example, a biphenyl group may be interpreted as an aryl group or a phenyl group substituted with a phenyl group. If a compound or a group unsubstituted, then the compound or group lacks a substituent.

As used herein, the term “adjacent group” may mean a any two functional groups or substituents bonded to two adjacent atoms optionally formed into a ring, two adjacent functional groups or substituents bonded to a single atom, or a group or substituent sterically positioned at the nearest position to a corresponding group or substituent. For example, in 1,2-dimethylbenzene, two methyl groups may be interpreted as “adjacent groups” to each other, and in 1,1-diethylcyclopentane, two ethyl groups may be interpreted as “adjacent groups” to each other.

As used herein, the halogen atom may be a fluorine atom, a chlorine atom, a bromine atom or an iodine atom, or a corresponding radical.

As used herein, the term “atom” may mean an element or its corresponding radical bonded to one or more other atoms.

As used herein, the term “alkyl” may mean a paraffinic hydrocarbon group optionally derived from an alkane by dropping one hydrogen from the formula, and may be linear, branched or cyclic. The number of carbon atoms of the alkyl may be 1 to 50, 1 to 30, 1 to 20, 1 to 10, or 1 to 6. Examples of the alkyl may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

As used herein, the hydrocarbon ring group means an optional functional group or substituent derived from an aliphatic hydrocarbon ring. The hydrocarbon ring group may be a saturated hydrocarbon ring group of 5 to 20 carbon atoms for forming a ring.

As used herein, the aryl group means an optional functional group or substituent whose ring structure may have the characteristics of benzene, naphthalene, phenanthrene, anthracene, etc. The aryl group may be a monocyclic aryl group or a polycyclic aryl group. The number of carbon atoms for forming a ring of the aryl group may be 6 to 40, 6 to 30, 6 to 20, 6 to 15, 16 to 40, 16 to 30, or 15 to 20. Examples of the aryl group may include phenyl, naphthyl, anthracenyl, phenanthryl, biphenyl, terphenyl, quaterphenyl, quinqphenyl, sexiphenyl, triphenylenyl, pyrenyl, benzofluoranthenyl, chrysenyl, etc., without limitation. As used herein, a fluorenyl group including SP³ hybrid carbon as ring-forming carbon is not defined as an aryl group. As used herein, the abbreviation “Ph” means a phenyl group.

As used herein, the heterocyclic group may be a closed-ring structure usually of five or six members that one or more of the atoms in the ring is an element other than carbon and may include one or more among B, O, N, P, Si and S as heteroatoms. If the heterocyclic group includes two or more heteroatoms, two or more heteroatoms may be the same or different. The heterocyclic group may be a monocyclic heterocyclic group or a polycyclic heterocyclic group, including a heteroaryl group. The number of carbons for forming a ring of the heteroaryl group may be 2 to 30, 2 to 20, 2 to 12, or 2 to 10.

As used herein, the alicyclic heterocyclic group may be characterized by closed ringed structures typically carbon atoms and saturated, and may include one or more among B, O, N, P, Si and S as heteroatoms. The number of carbon atoms for forming a ring of the aliphatic heterocyclic group may be 2 to 30, 2 to 20, or 2 to 15, 2 to 12, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

As used herein, the number of carbon atoms for forming a ring of the heteroaryl group may be 2 to 30, 2 to 20, 2 to 15, 2 to 12, or 2 to 10. Examples of the heteroaryl group may include thiophene, furan, pyrrole, imidazole, thiazole, oxazole, oxadiazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, isooxazole, thiadiazole, phenothiazine, dibenzosilole, dibenzofuran, etc., without limitation.

As used herein, the definition of the aryl group may also be applicable to an arylene group except that the arylene group is a divalent group. The definition of the heteroaryl group may be also applicable to an heteroarylene group except that the heteroarylene group is a divalent group.

As used herein, the silyl group includes an alkyl silyl group and an aryl silyl group. Examples of the silyl group may include trimethylsilyl, triethylsilyl, t-butyldimethylsilyl, vinyldimethylsilyl, propyldimethylsilyl, triphenylsilyl, diphenylsilyl, phenylsilyl, etc. However, the exemplary embodiments are not limited thereto.

As used herein, the number of the carbon atoms of the amino group is not specifically limited, but may be 1 to 30. The amino group may include an alkyl amino group, an aryl amino group, or a heteroaryl amino group. Examples of the amino group include a methylamino group, a dimethylamino group, a phenylamino group, a diphenylamino group, a naphthylamino group, a 9-methyl-anthracenylamino group, a triphenylamino group, etc., without limitation.

As used herein, the alkenyl group may be a linear chain or a branched chain. The number of carbon atoms is not specifically limited but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

As used herein, the number of carbon atoms of the amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation.

As used herein, an alkyl group or substituent of the alkylthio group, alkylsulfoxy group, alkylaryl group, alkylamino group, alkylboron group, alkyl silyl group, and alkyl amine group may have the same definition as above-described alkyl group.

As used herein, the aryl group or substituent of the aryloxy group, arylthio group, arylsulfoxy group, aryl amino group, arylboron group, and aryl silyl group may have the same definition as above-described aryl group.

As used herein, the direct linkage may mean a single bond.

As used herein, “ [symbol] ” means a connected position.

Various exemplary embodiments are described herein with reference to sectional and/or exploded illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

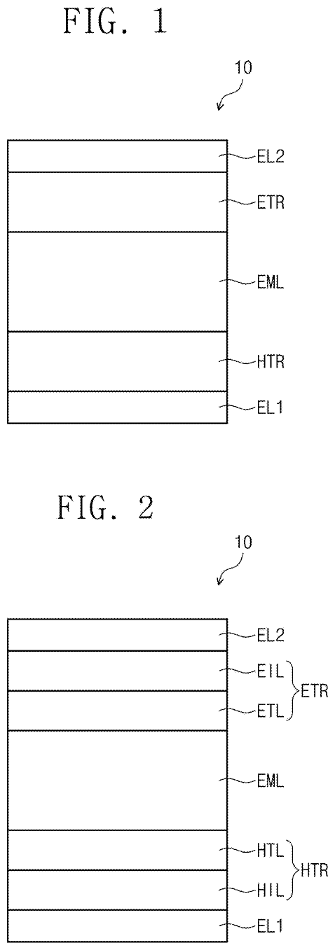

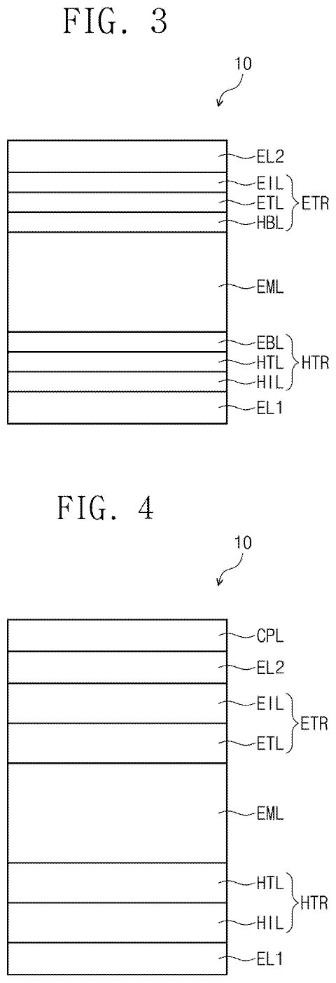

FIG. 1 is a schematic cross-sectional diagram of an exemplary embodiment of a luminescence device constructed according to principles of the invention. FIG. 2 is a schematic cross-sectional diagram of another exemplary embodiment of a luminescence device constructed according to principles of the invention. FIG. 3 is a schematic cross-sectional diagram of yet another exemplary embodiment of a luminescence device constructed according to principles of the invention. FIG. 4 is a schematic cross-sectional diagram of a further exemplary embodiment of a luminescence device constructed according to principles of the invention.

The luminescence device 10 according to some exemplary embodiments may include a first electrode EL1, a hole transport region HTR, an emission layer EML, an electron transport region ETR, and a second electrode EL2 stacked in order.

Referring to FIGS. 1 to 4, in a luminescence device 10 according to some exemplary embodiments, a first electrode EL1 and a second electrode EL2 are oppositely disposed, and between the first electrode EL1 and the second electrode EL2, an emission layer EML may be disposed.

In addition, the luminescence device 10 further includes a plurality of functional groups between the first electrode EL1 and the second electrode EL2 in addition to the emission layer EML. The plurality of the functional groups may include a hole transport region HTR and an electron transport region ETR. That is, the luminescence device 10 may include the first electrode EL1, the hole transport region HTR, the emission layer EML, the electron transport region ETR, and the second electrode EL2, stacked in order. In addition, the luminescence device 10 may include a capping layer CPL that may be disposed on the second electrode EL2.

The luminescence device 10 may include exemplary embodiments of an amine compound, which will be explained below, in the hole transport region HTR disposed between the first electrode EL1 and the second electrode EL2. However, the exemplary embodiments are not limited thereto, and the luminescence device 10 may include an amine compound in the emission layer EML or the electron transport region ETR, which are functional groups disposed between the first electrode EL1 and the second electrode EL2, or include an amine compound in the capping layer CPL disposed on the second electrode EL2, in addition to the hole transport region HTR. The emission layer EML may include an organic light-emitting material and/or an inorganic light-emitting material such as a quantum dot. Hereinafter, an organic electroluminescence device in which the emission layer EML includes an organic light-emitting material will be explained for convenience of illustration.

In comparison to FIG. 1, FIG. 2 shows the cross-sectional view of a luminescence device 10 of an exemplary embodiment, where a hole transport region HTR includes a hole injection layer HIL and a hole transport layer HTL, and an electron transport region ETR includes an electron injection layer EIL and an electron transport layer ETL. In addition, FIG. 3 shows the cross-sectional view of a luminescence device 10 of an exemplary embodiment, where a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison with FIG. 2, FIG. 4 shows the cross-sectional view of a luminescence device 10 of an exemplary embodiment, including a capping layer CPL disposed on the second electrode EL2.

The first electrode EL1 has conductivity. The first electrode EL1 may be formed using a metal alloy or a conductive compound. The first electrode EL1 may be an anode. Also, the first electrode EL1 may be a pixel electrode. The first electrode EL1 may be a transmissive electrode, a transflective electrode, or a reflective electrode. If the first electrode EL1 is the transmissive electrode, the first electrode EL1 may include a transparent metal oxide, for example, an indium tin oxide (ITO), an indium zinc oxide (IZO), a zinc oxide (ZnO), and an indium tin zinc oxide (ITZO), etc. If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca, LiF/Al, Mo, and Ti, a compound thereof, or a mixture thereof (for example, a mixture of Ag and Mg). Alternatively, the first electrode EL1 may have a structure including a plurality of layers including a reflective layer or a transflective layer formed using the above materials, and a transmissive conductive layer formed using an ITO, an IZO, a ZnO, and/or an ITZO, etc. For example, the first electrode EL1 may include a three-layer structure of ITO/Ag/ITO. However, the exemplary embodiments are not limited thereto. The thickness of the first electrode EL1 may be from about 1,000 Å to about 10,000 Å, for example, from about 1,000 Å to about 3,000 Å.

The hole transport region HTR is disposed on the first electrode EL1. The hole transport region HTR may include at least one of a hole injection layer HIL, a hole transport layer HTL, a hole buffer layer, or an electron blocking layer EBL.

The hole transport region HTR may have a single layer formed using a single material, a single layer formed using a plurality of different materials, or a multilayer structure including a plurality of layers formed using a plurality of different materials.

For example, the hole transport region HTR may have a single layer structure of the hole injection layer HIL or the hole transport layer HTL, or a single layer structure formed using a hole injection material and a hole transport material. In addition, the hole transport region HTR may have a structure of a single layer formed using a plurality of different materials, or a structure stacked from the first electrode EL1 of hole injection layer HIL/hole transport layer HTL, hole injection layer HIL/hole transport layer HTL/hole buffer layer, hole injection layer HIL/hole buffer layer, hole transport layer HTL/hole buffer layer, or hole injection layer HIL/hole transport layer HTL/electron blocking layer EBL, without limitation.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may include an amine compound represented by the following Formula 1:

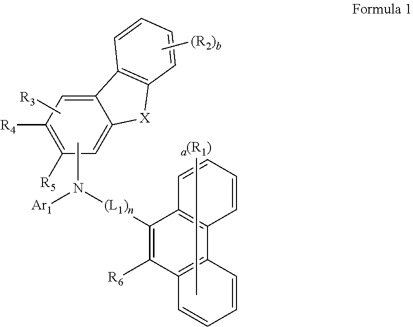

In Formula 1, X may be O or S.

R1 to R5 may be each independently a hydrogen atom, a deuterium atom, a halogen atom, a silyl group, an alkyl group, an aryl group, or a heteroaryl group. The halogen atom may be a fluorine atom or a chlorine atom. The silyl group may be a substituted or unsubstituted silyl group. The substituted silyl group may be an alkyl silyl group or an aryl silyl group. The alkyl group may be a substituted or unsubstituted alkyl group of 1 to 20 carbon atoms. The aryl group may be a substituted or unsubstituted aryl group of 6 to 40 carbon atoms for forming a ring. The heteroaryl group may be a substituted or unsubstituted heteroaryl group of 2 to 40 carbon atoms for forming a ring.

For example, R1 to R₅ may be each independently a hydrogen atom, a deuterium atom, a methyl group, an ethyl group, a n-propyl group, an iso-propyl group, a n-butyl group, an iso-butyl group, a tert-butyl group, a fluorine atom, a triphenylsilyl group, a substituted or unsubstituted phenyl group.

R4 and R5 may be hydrogen atoms or deuterium atoms.

R6 may be a hydrogen atom, or a deuterium atom,

L1 may be an arylene group, or a heteroarylene group. The arylene group may be a substituted or unsubstituted arylene group of 6 to 40 carbon atoms for forming a ring. The heteroarylene group may be a substituted or unsubstituted heteroarylene group of 2 to 40 carbon atoms for forming a ring.

Ar1 may be an aryl group, or a heteroaryl group. The aryl group may be a substituted or unsubstituted aryl group of 6 to 40 carbon atoms for forming a ring. The heteroaryl group may be a substituted or unsubstituted heteroaryl group of 2 to 12 carbon atoms for forming a ring.

If X is O and Ar1 is a substituted or unsubstituted aryl group, carbon number for forming a ring of Ar1 may be 16 to 40.

The variables “a” may be an integer of 0 to 8, “b” may be an integer of 0 to 4, and/or “n” may be an integer of 1 to 3. For example, the variables “a” may be 0, 1, or 2, “b” may be 1, and/or “n” may be 1. As a further example, “a” may be an integer of 0 to 2 and/or “b” may be 0 or 1. If “a” is an integer of 2 or more, a plurality of R1 groups may be the same or different. If “a” is 2, R1 groups may be substituted at carbon atoms of positions 2 and 7 of a phenanthryl skeleton. If “b” is an integer of 2 or more, a plurality of R2 groups may be the same or different. If “n” is an integer of 2 or more, a plurality of L1 groups may be the same or different.

Ar1 may be represented by the following Formula 2:

[symbol]

ₚ(Ar₁₁)—Ar₁₂ Formula 2

In Formula 2, Ar11 may be an arylene group or a heteroarylene group. The arylene group may be a substituted or unsubstituted arylene group of 6 to 40 carbon atoms for forming a ring, and the heteroarylene group may be a substituted or unsubstituted heteroarylene group of 2 to 12 carbon atoms for forming a ring.

Ar12 may be an aryl group or a heteroaryl group. The aryl group may be a substituted or unsubstituted aryl group of 6 to 40 carbon atoms for forming a ring, or a substituted or unsubstituted aryl group of 16 to 40 carbon atoms for forming a ring. If X is O and Ar12 is a substituted or unsubstituted aryl group, the carbon number for forming a ring of Ar12 may be 16 to 40.

The heteroaryl group may be a substituted or unsubstituted heteroaryl group of 2 to 12 carbon atoms for forming a ring. The variable “p” may be 0 or 1.

Ar1 may be represented by the following Formulae 1-1 to 1-10:

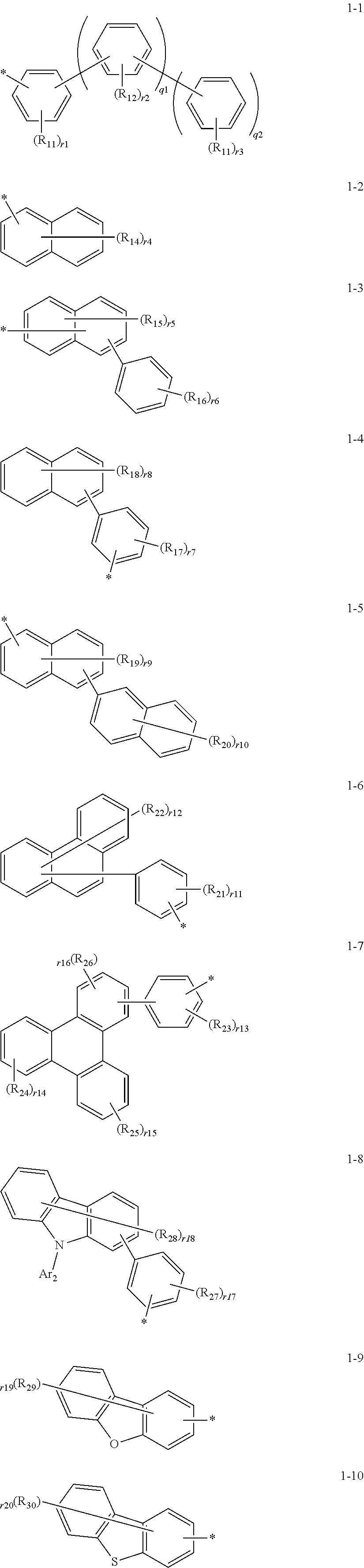

In Formulae 1-1 to 1-10, R₁₁ to R₃₀ may be each independently a hydrogen atom, a deuterium atom, a halogen atom, a silyl group, an alkyl group, an aryl group, or a heteroaryl group. The halogen atom may be a fluorine atom or a chlorine atom. The silyl group may be a substituted or unsubstituted silyl group. The substituted silyl group may be an alkyl silyl group or an aryl silyl group. The alkyl group may be a substituted or unsubstituted alkyl group of 1 to 20 carbon atoms. The aryl group may be a substituted or unsubstituted aryl group of 6 to 40 carbon atoms for forming a ring. The heteroaryl group may be a substituted or unsubstituted heteroaryl group of 2 to 40 carbon atoms for forming a ring.

Ar₂ may be an alkyl group, an aryl group, or a heteroaryl group. The alkyl group may be a substituted or unsubstituted alkyl group of 1 to 20 carbon atoms. The aryl group may be a substituted or unsubstituted aryl group of 6 to 40 carbon atoms for forming a ring. The heteroaryl group may be a substituted or unsubstituted heteroaryl group of 2 to 40 carbon atoms for forming a ring.

The variables q1 and q2 may be each independently 0 or 1.

The variables r1, r7, r11, and r13 to r17 may be each independently an integer of 0 to 4. The variables r2, r3, and r6 may be each independently an integer of 0 to 5. The variables r4, r8, r10, and r18 to r20 may be each independently an integer of 0 to 7. The variables r5 and r9 may be each independently an integer of 0 to 6, and/or r12 may be an integer of 0 to 9. If r1 is an integer of 2 or more, a plurality of Ru groups may be the same or different. The same explanation on r1 may be applied to r12 to r20.

Ar₁ may be represented by the following Formulae 1-11 to 1-20:

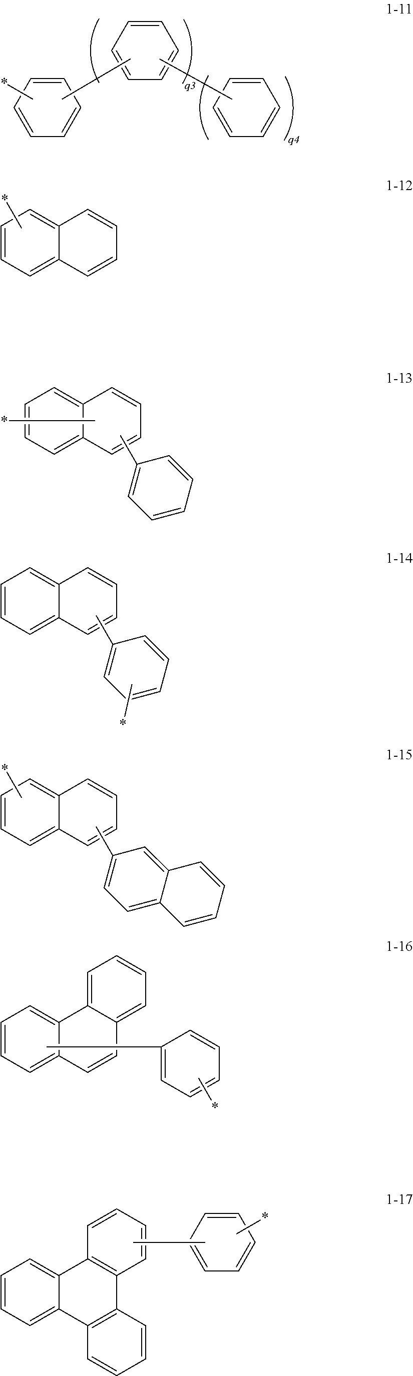

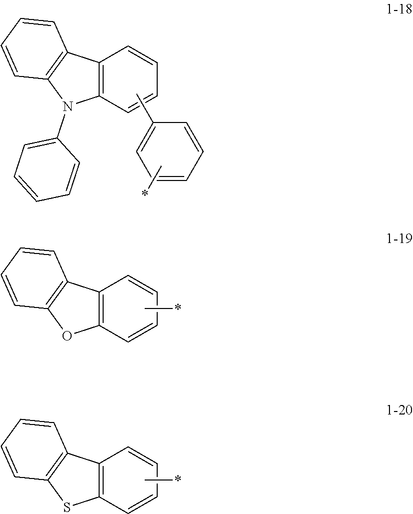

Formulae 1-11 to 1-20 are embodied chemical formulae of 1-1 to 1-10. In Formulae 1-11, q3 and q4 may be each independently 0 or 1.

The variable L₁ may be represented by the following Formulae 2-1 to 2-4:

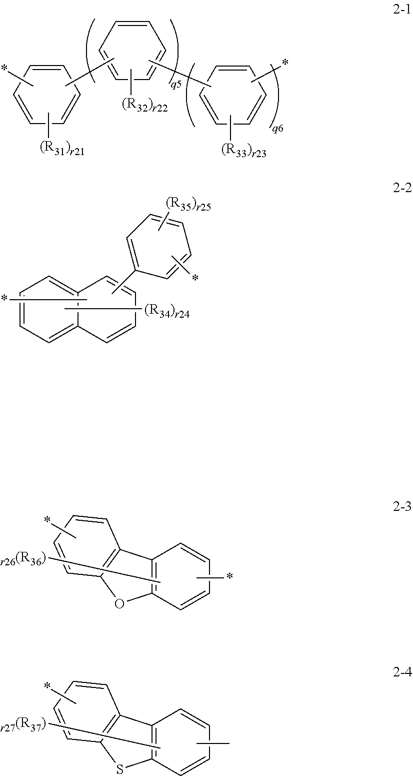

In Formulae 2-1 to 2-4, R₃₁ to R₃₇ may be each independently a hydrogen atom, a deuterium atom, a halogen atom, a silyl group, an alkyl group, an aryl group, or a heteroaryl group. The silyl group may be a substituted or unsubstituted silyl group. The silyl group may be an alkyl silyl group or an aryl silyl group. The alkyl group may be a substituted or unsubstituted alkyl group of 1 to 20 carbon atoms. The aryl group may be a substituted or unsubstituted aryl group of 6 to 40 carbon atoms for forming a ring. The heteroaryl group may be a substituted or unsubstituted heteroaryl group of 2 to 40 carbon atoms for forming a ring.

The variables q5 and q6 may be each independently 0 or 1. The variables r21 to r23, and r25 may be each independently an integer of 0 to 4. The variables r24, r26, and r27 may be each independently an integer of 0 to 6. If the variable r21 is an integer of 2 or more, a plurality of R₃₁ groups may be the same or different. The same explanation on r1 may be applied to r22 to r27.

The variable L₁ may be represented by the following Formulae 2-11 to 2-14:

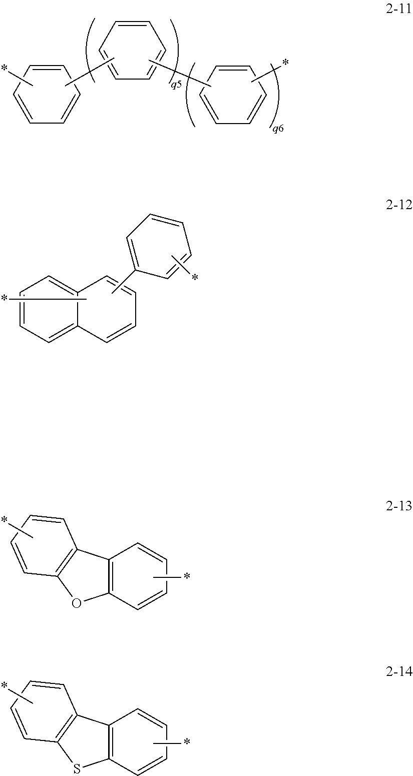

In Formulae 2-11, q5 and q6 may be each independently 0 or 1.

Formula 1 may be represented by the following Formula 1-1:

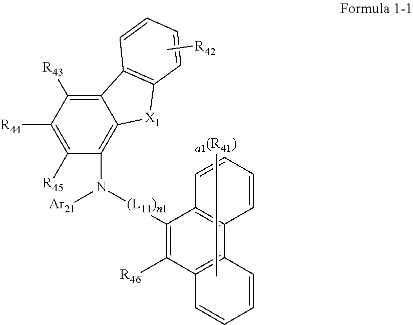

In Formula 1-1, X₁ may be O or S.

R₄₁, and R₄₂ may be each independently a hydrogen atom, a deuterium atom, a halogen atom, a silyl group, an alkyl group, an aryl group, or a heteroaryl group. The silyl group may be a substituted or unsubstituted silyl group. The alkyl group may be a substituted or unsubstituted alkyl group of 1 to 20 carbon atoms. The aryl group may be a substituted or unsubstituted aryl group of 6 to 40 carbon atoms for forming a ring. The heteroaryl group may be a substituted or unsubstituted heteroaryl group of 2 to 40 carbon atoms for forming a ring.

R₄₃ to R₄₆ may be each independently a hydrogen atom, or a deuterium atom.

L₁₁ may be an arylene group or a heteroarylene group. The arylene group may be a substituted or unsubstituted arylene group of 6 to 40 carbon atoms for forming a ring. The heteroarylene group may be a substituted or unsubstituted heteroarylene group of 2 to 40 carbon atoms for forming a ring.

Ar₂₁ may be an aryl group or a heteroaryl group. The aryl group may be a substituted or unsubstituted aryl group of 6 to 40 carbon atoms for forming a ring. The heteroaryl group may be a substituted or unsubstituted heteroaryl group of 2 to 12 carbon atoms for forming a ring.

If X₁ is O and Ar₂₁ is a substituted or unsubstituted aryl group, carbon number for forming a ring of Ar₂₁ may be 16 to 40.

The variable “a1” may be an integer of 0 to 2, and/or “n1” may be an integer of 1 to 3.

Formula 1 may be represented by the following Formula 1-2:

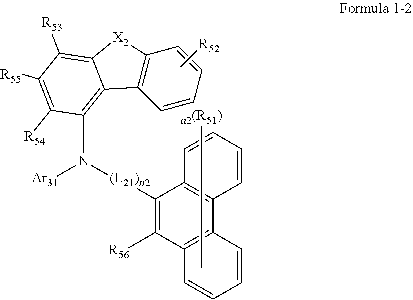

In Formula 1-2, X₂ may be O or S.

R₅₁, and R₅₂ may be each independently a hydrogen atom, a deuterium atom, a halogen atom, a silyl group, an alkyl group, an aryl group, or a heteroaryl group. The silyl group may be a substituted or unsubstituted silyl group. The alkyl group may be a substituted or unsubstituted alkyl group of 1 to 20 carbon atoms. The aryl group may be a substituted or unsubstituted aryl group of 6 to 40 carbon atoms for forming a ring. The heteroaryl group may be a substituted or unsubstituted heteroaryl group of 2 to 40 carbon atoms for forming a ring.

R₅₃ to R₅₆ may be each independently a hydrogen atom or a deuterium atom.

L₂₁ may be an arylene group or a heteroarylene group. The arylene group may be a substituted or unsubstituted arylene group of 6 to 40 carbon atoms for forming a ring. The heteroarylene group may be a substituted or unsubstituted heteroarylene group of 2 to 40 carbon atoms for forming a ring.

Ar₃₁ may be an aryl group or a heteroaryl group. The aryl group may be a substituted or unsubstituted aryl group of 6 to 40 carbon atoms for forming a ring. The heteroaryl group may be a substituted or unsubstituted heteroaryl group of 2 to 12 carbon atoms for forming a ring.

If X₂ is O and Ar₃₁ is a substituted or unsubstituted aryl group, carbon number for forming a ring of Ar₃₁ may be 16 to 40.

The variable “a2” may be an integer of 0 to 2 and/or “n2” may be an integer of 1 to 3.

The above-described amine compound of some exemplary embodiments may be a monoamine compound. The amine compound may further include a cyclic amine. If the amine compound is a diamine compound, one acyclic amine and one cyclic amine may be included. For example, if the amine compound of some exemplary embodiments is a diamine compound, a substituted or unsubstituted carbazole group may be included.

Formula 1 may be any one selected among the compounds represented in the following Compound Group A to Compound Group D:

Compound Group A

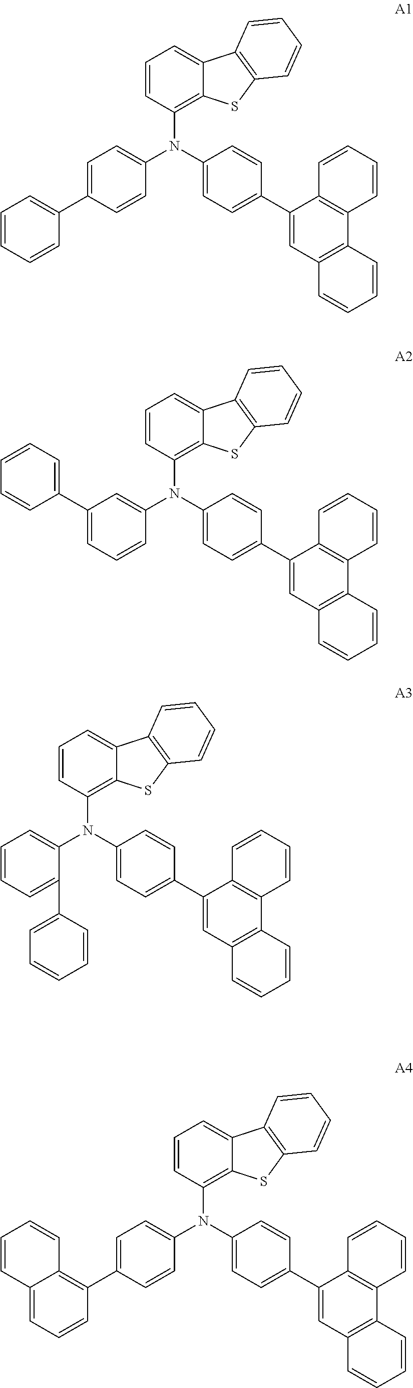 [SMILES: c1ccc(-c2ccc(N(c3ccc(-c4cc5ccccc5c5ccccc45)cc3)c3cccc4c3sc3ccccc34)cc2)cc1 ; c1ccc(-c2cccc(N(c3ccc(-c4cc5ccccc5c5ccccc45)cc3)c3cccc4c3sc3ccccc34)c2)cc1 ; c1ccc(-c2ccccc2N(c2ccc(-c3cc4ccccc4c4ccccc34)cc2)c2cccc3c2sc2ccccc23)cc1 ; c1ccc2c(-c3ccc(N(c4ccc(-c5cc6ccccc6c6ccccc56)cc4)c4cccc5c4sc4ccccc45)cc3)cccc2c1]

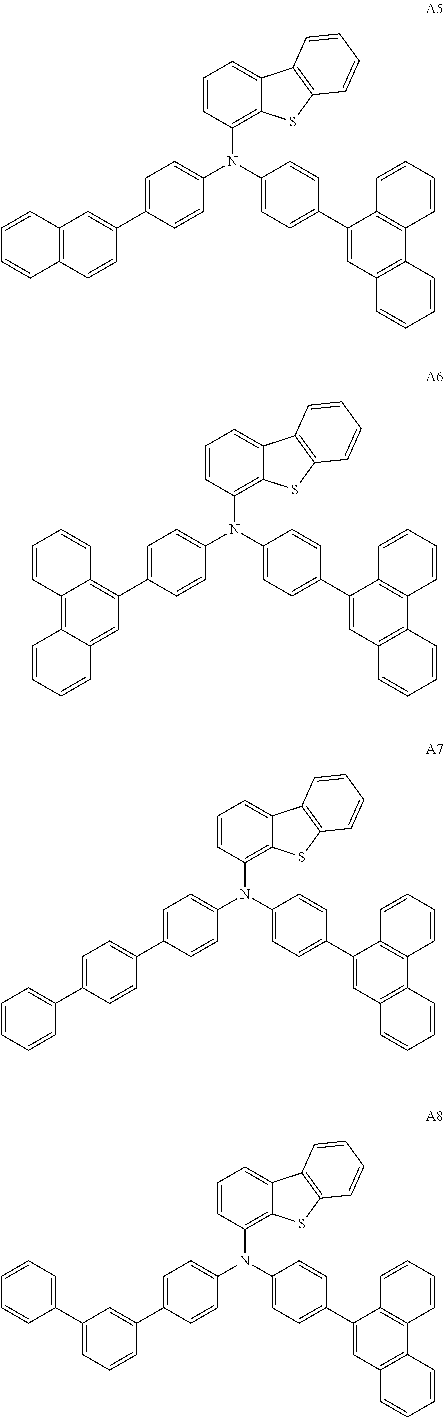 [SMILES: c1ccc2cc(-c3ccc(N(c4ccc(-c5cc6ccccc6c6ccccc56)cc4)c4cccc5c4sc4ccccc45)cc3)ccc2c1 ; c1ccc2c(c1)cc(-c1ccc(N(c3ccc(-c4cc5ccccc5c5ccccc45)cc3)c3cccc4c3sc3ccccc34)cc1)c1ccccc12 ; c1ccc(-c2ccc(-c3ccc(N(c4ccc(-c5cc6ccccc6c6ccccc56)cc4)c4cccc5c4sc4ccccc45)cc3)cc2)cc1 ; c1ccc(-c2cccc(-c3ccc(N(c4ccc(-c5cc6ccccc6c6ccccc56)cc4)c4cccc5c4sc4ccccc45)cc3)c2)cc1]

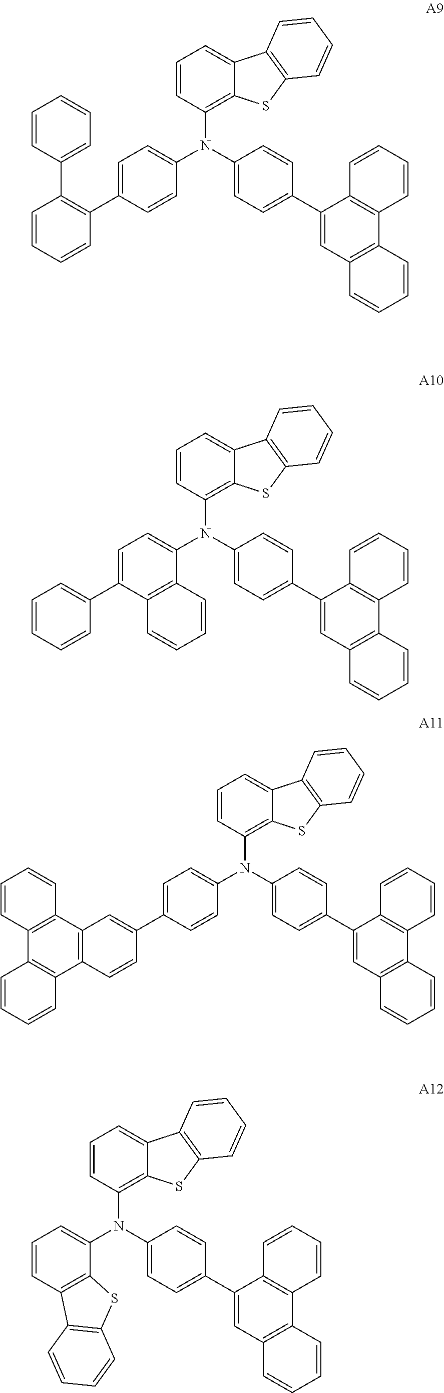 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)c3ccccc23)cc1 ; c1ccc2c(c1)cc(-c1ccc(N(c3ccc(-c4ccc5c6ccccc6c6ccccc6c5c4)cc3)c3cccc4c3sc3ccccc34)cc1)c1ccccc12 ; c1ccc2c(c1)cc(-c1ccc(N(c3cccc4c3sc3ccccc34)c3cccc4c3sc3ccccc34)cc1)c1ccccc12]

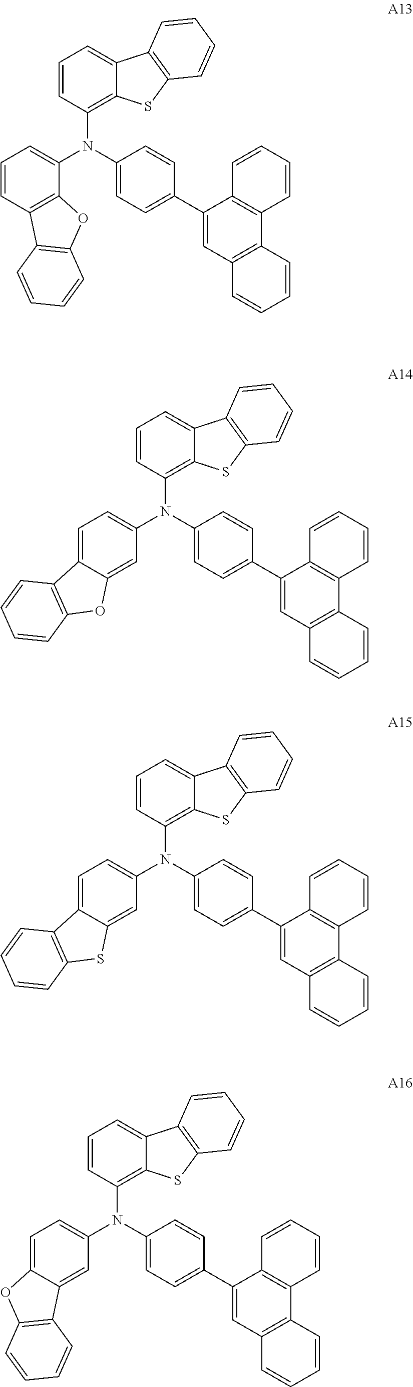 [SMILES: c1ccc2c(c1)cc(-c1ccc(N(c3cccc4c3oc3ccccc34)c3cccc4c3sc3ccccc34)cc1)c1ccccc12 ; c1ccc2c(c1)cc(-c1ccc(N(c3ccc4c(c3)oc3ccccc34)c3cccc4c3sc3ccccc34)cc1)c1ccccc12 ; c1ccc2c(c1)cc(-c1ccc(N(c3ccc4c(c3)sc3ccccc34)c3cccc4c3sc3ccccc34)cc1)c1ccccc12 ; c1ccc2c(c1)cc(-c1ccc(N(c3ccc4oc5ccccc5c4c3)c3cccc4c3sc3ccccc34)cc1)c1ccccc12]

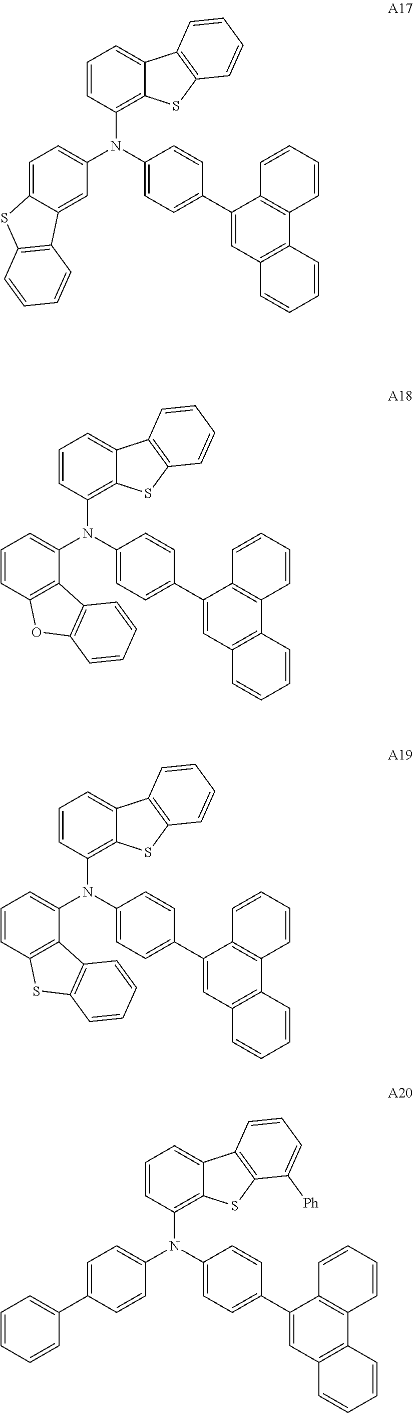 [SMILES: c1ccc2c(c1)cc(-c1ccc(N(c3ccc4sc5ccccc5c4c3)c3cccc4c3sc3ccccc34)cc1)c1ccccc12 ; c1ccc2c(c1)cc(-c1ccc(N(c3cccc4c3sc3ccccc34)c3cccc4oc5ccccc5c34)cc1)c1ccccc12 ; c1ccc2c(c1)cc(-c1ccc(N(c3cccc4c3sc3ccccc34)c3cccc4sc5ccccc5c34)cc1)c1ccccc12 ; c1ccc(-c2ccc(N(c3ccc(-c4cc5ccccc5c5ccccc45)cc3)c3cccc4c3sc3c(-c5ccccc5)cccc34)cc2)cc1]

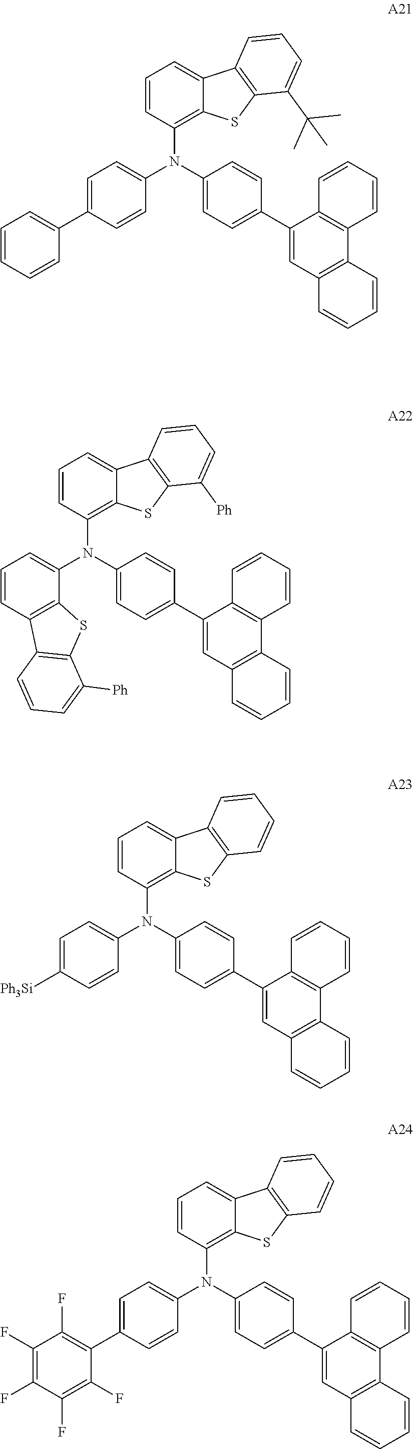

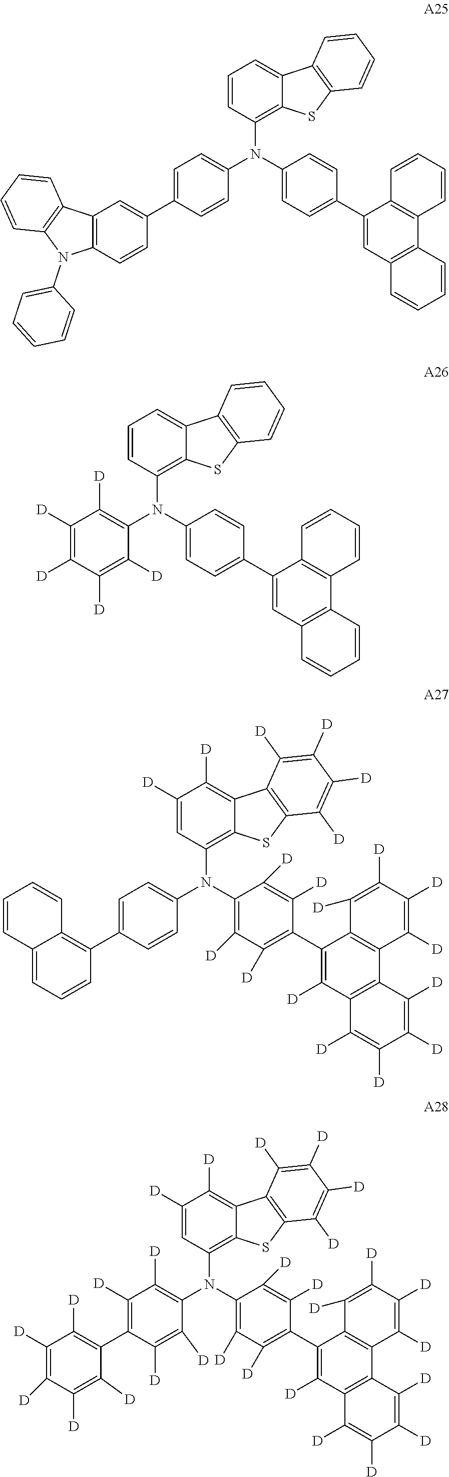

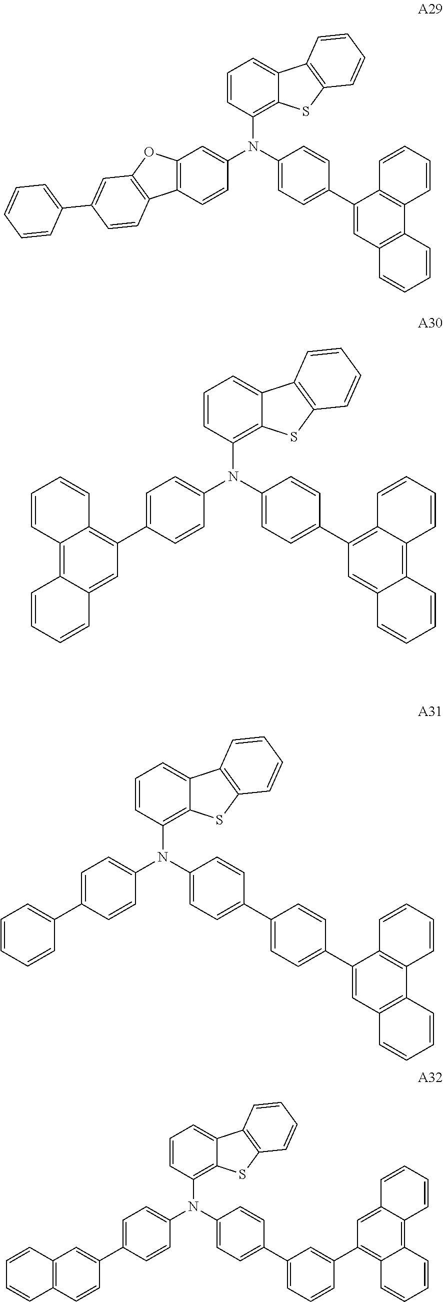 [SMILES: c1ccc(-c2ccc3c(c2)oc2cc(N(c4ccc(-c5cc6ccccc6c6ccccc56)cc4)c4cccc5c4sc4ccccc45)ccc23)cc1 ; c1ccc2c(c1)cc(-c1ccc(N(c3ccc(-c4cc5ccccc5c5ccccc45)cc3)c3cccc4c3sc3ccccc34)cc1)c1ccccc12 ; c1ccc(-c2ccc(N(c3ccc(-c4ccc(-c5cc6ccccc6c6ccccc56)cc4)cc3)c3cccc4c3sc3ccccc34)cc2)cc1 ; c1cc(-c2ccc(N(c3ccc(-c4ccc5ccccc5c4)cc3)c3cccc4c3sc3ccccc34)cc2)cc(-c2cc3ccccc3c3ccccc23)c1]

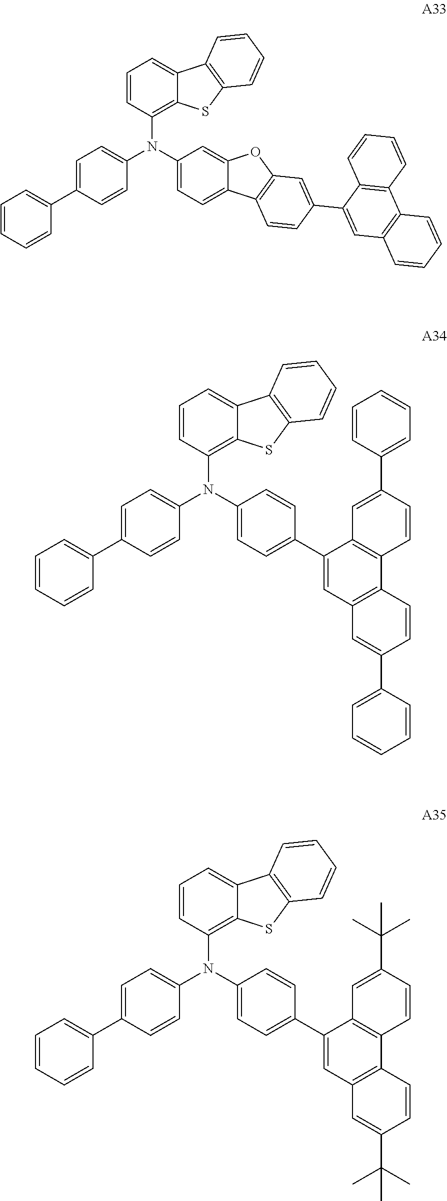 [SMILES: c1ccc(-c2ccc(N(c3ccc4c(c3)oc3cc(-c5cc6ccccc6c6ccccc56)ccc34)c3cccc4c3sc3ccccc34)cc2)cc1 ; c1ccc(-c2ccc(N(c3ccc(-c4cc5cc(-c6ccccc6)ccc5c5ccc(-c6ccccc6)cc45)cc3)c3cccc4c3sc3ccccc34)cc2)cc1 ; CC(C)(C)c1ccc2c(c1)cc(-c1ccc(N(c3ccc(-c4ccccc4)cc3)c3cccc4c3sc3ccccc34)cc1)c1cc(C(C)(C)C)ccc12]

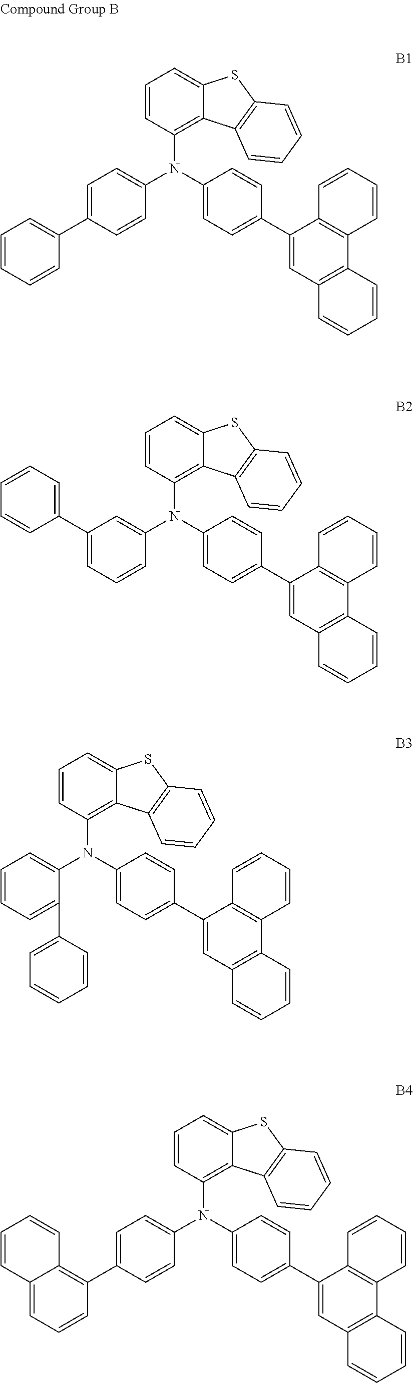 [SMILES: c1ccc(-c2ccc(N(c3ccc(-c4cc5ccccc5c5ccccc45)cc3)c3cccc4sc5ccccc5c34)cc2)cc1 ; c1ccc(-c2cccc(N(c3ccc(-c4cc5ccccc5c5ccccc45)cc3)c3cccc4sc5ccccc5c34)c2)cc1 ; c1ccc(-c2ccccc2N(c2ccc(-c3cc4ccccc4c4ccccc34)cc2)c2cccc3sc4ccccc4c23)cc1 ; c1ccc2c(-c3ccc(N(c4ccc(-c5cc6ccccc6c6ccccc56)cc4)c4cccc5sc6ccccc6c45)cc3)cccc2c1]

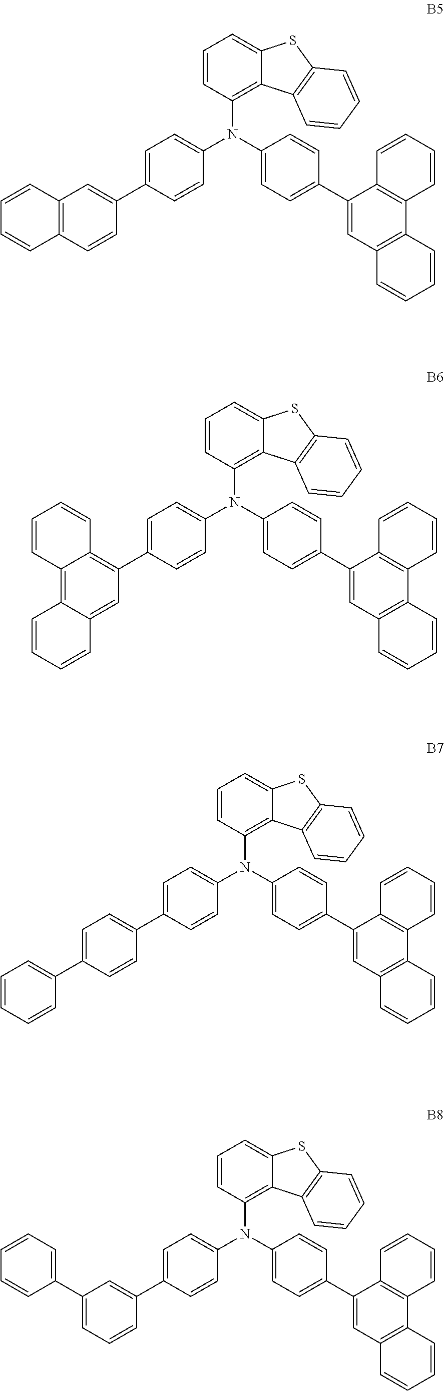 [SMILES: c1ccc2cc(-c3ccc(N(c4ccc(-c5cc6ccccc6c6ccccc56)cc4)c4cccc5sc6ccccc6c45)cc3)ccc2c1 ; c1ccc2c(c1)cc(-c1ccc(N(c3ccc(-c4cc5ccccc5c5ccccc45)cc3)c3cccc4sc5ccccc5c34)cc1)c1ccccc12 ; c1ccc(-c2ccc(-c3ccc(N(c4ccc(-c5cc6ccccc6c6ccccc56)cc4)c4cccc5sc6ccccc6c45)cc3)cc2)cc1 ; c1ccc(-c2cccc(-c3ccc(N(c4ccc(-c5cc6ccccc6c6ccccc56)cc4)c4cccc5sc6ccccc6c45)cc3)c2)cc1]

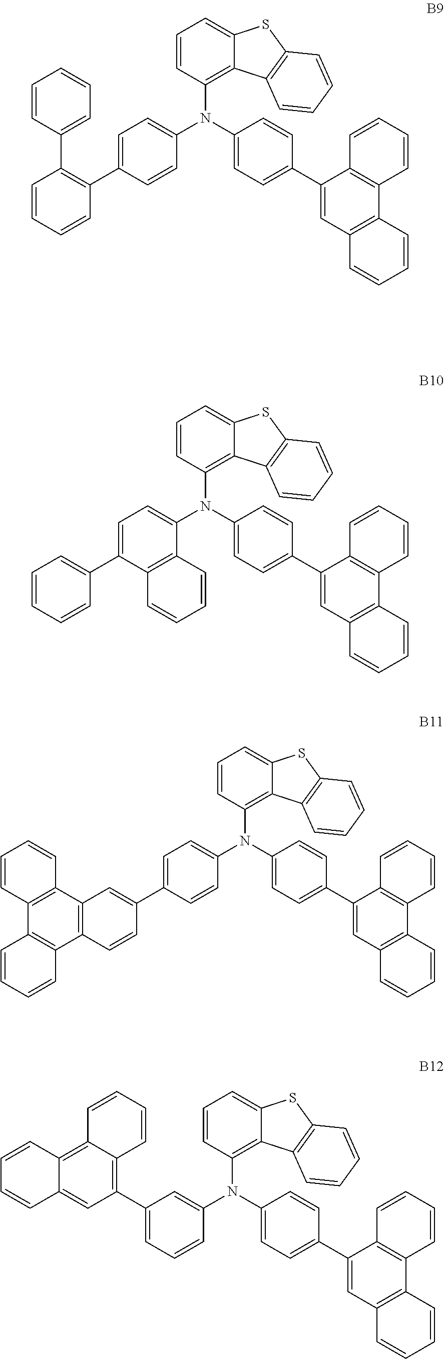 [SMILES: c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)c3ccccc23)cc1 ; c1ccc2c(c1)cc(-c1ccc(N(c3ccc(-c4ccc5c6ccccc6c6ccccc6c5c4)cc3)c3cccc4sc5ccccc5c34)cc1)c1ccccc12 ; c1cc(-c2cc3ccccc3c3ccccc23)cc(N(c2ccc(-c3cc4ccccc4c4ccccc34)cc2)c2cccc3sc4ccccc4c23)c1]

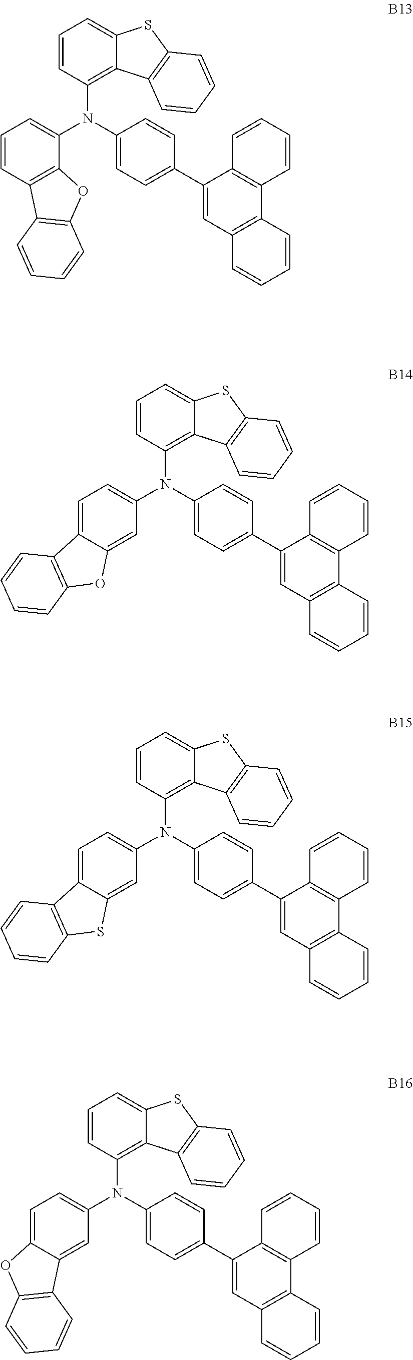 [SMILES: c1ccc2c(c1)cc(-c1ccc(N(c3cccc4c3oc3ccccc34)c3cccc4sc5ccccc5c34)cc1)c1ccccc12 ; c1ccc2c(c1)cc(-c1ccc(N(c3ccc4c(c3)oc3ccccc34)c3cccc4sc5ccccc5c34)cc1)c1ccccc12 ; c1ccc2c(c1)cc(-c1ccc(N(c3ccc4c(c3)sc3ccccc34)c3cccc4sc5ccccc5c34)cc1)c1ccccc12 ; c1ccc2c(c1)cc(-c1ccc(N(c3ccc4oc5ccccc5c4c3)c3cccc4sc5ccccc5c34)cc1)c1ccccc12]

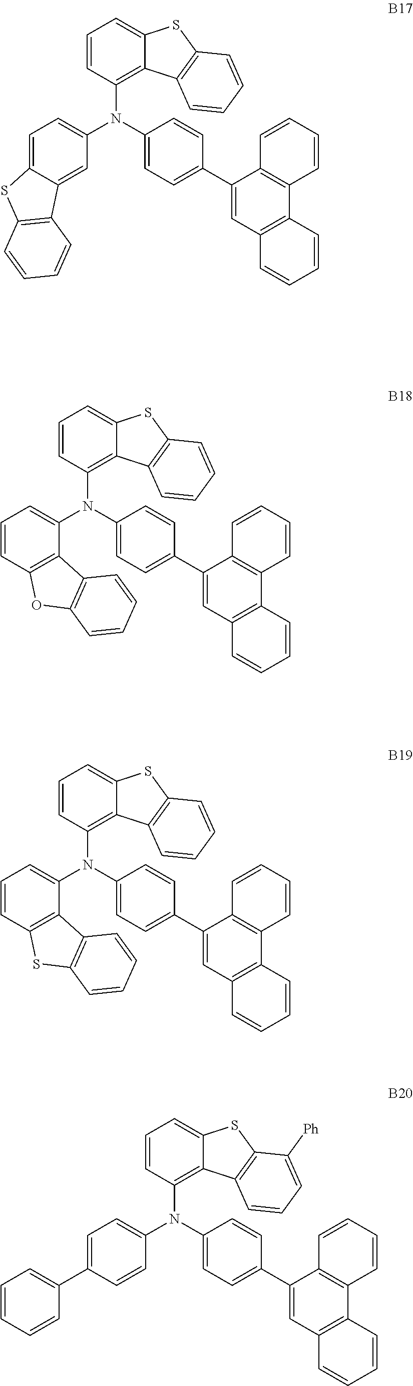 [SMILES: c1ccc2c(c1)cc(-c1ccc(N(c3ccc4sc5ccccc5c4c3)c3cccc4sc5ccccc5c34)cc1)c1ccccc12 ; c1ccc2c(c1)cc(-c1ccc(N(c3cccc4oc5ccccc5c34)c3cccc4sc5ccccc5c34)cc1)c1ccccc12 ; c1ccc2c(c1)cc(-c1ccc(N(c3cccc4sc5ccccc5c34)c3cccc4sc5ccccc5c34)cc1)c1ccccc12 ; c1ccc(-c2ccc(N(c3ccc(-c4cc5ccccc5c5ccccc45)cc3)c3cccc4sc5c(-c6ccccc6)cccc5c34)cc2)cc1]

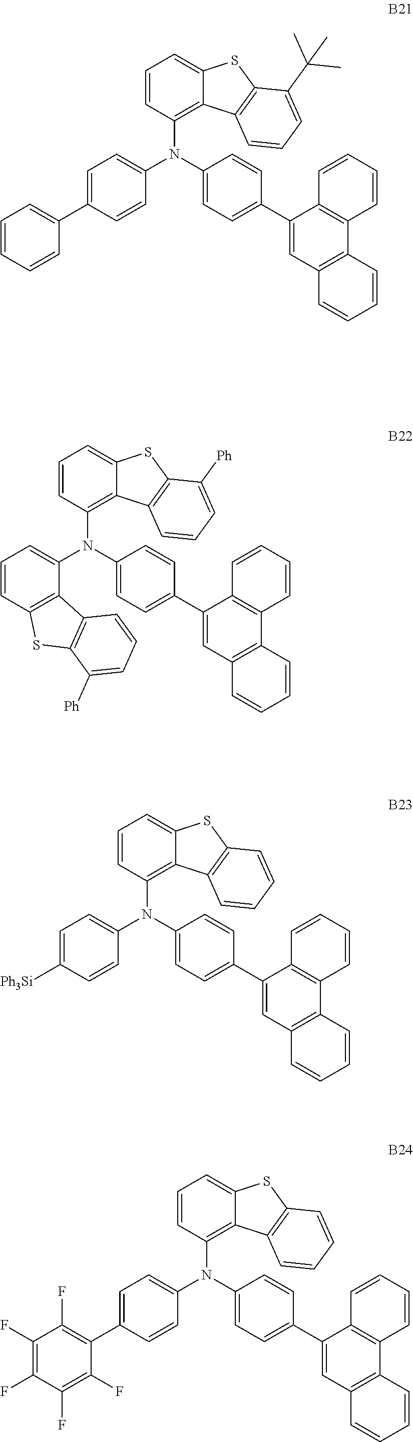

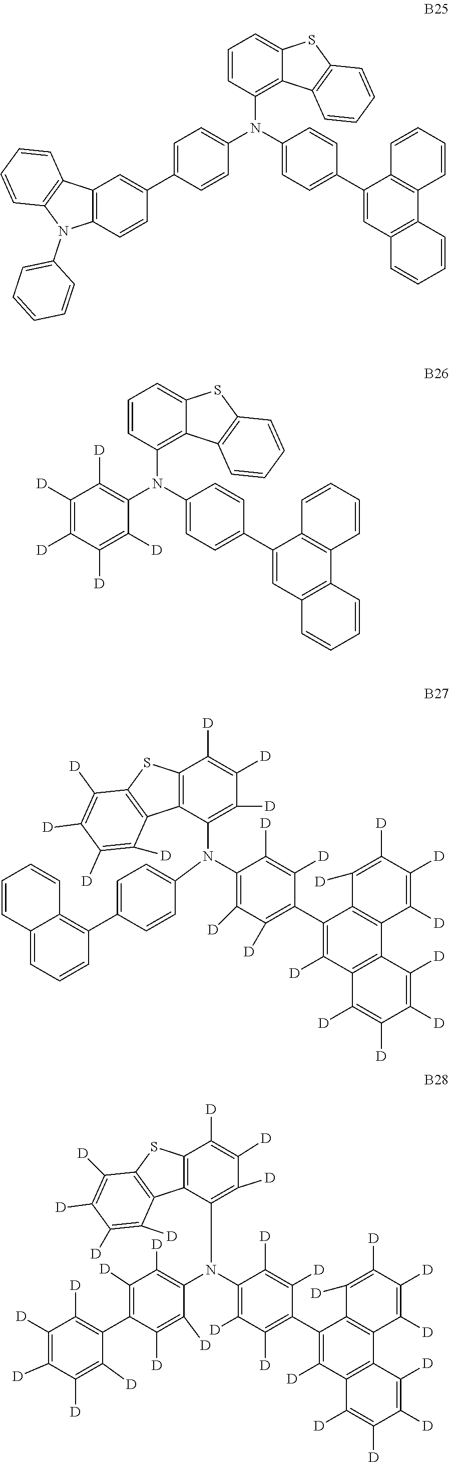

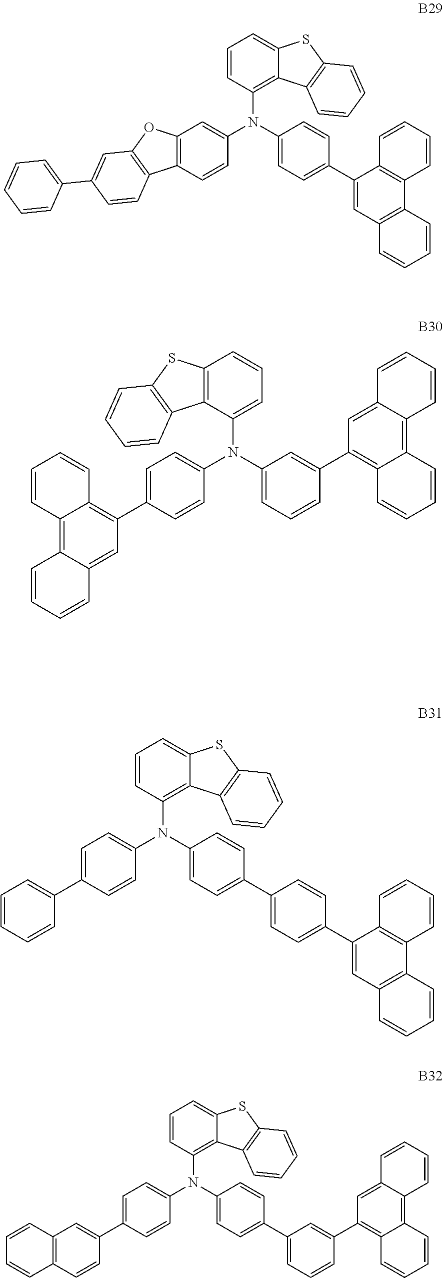 [SMILES: c1ccc(-c2ccc3c(c2)oc2cc(N(c4ccc(-c5cc6ccccc6c6ccccc56)cc4)c4cccc5sc6ccccc6c45)ccc23)cc1 ; c1cc(-c2cc3ccccc3c3ccccc23)cc(N(c2ccc(-c3cc4ccccc4c4ccccc34)cc2)c2cccc3sc4ccccc4c23)c1 ; c1ccc(-c2ccc(N(c3ccc(-c4ccc(-c5cc6ccccc6c6ccccc56)cc4)cc3)c3cccc4sc5ccccc5c34)cc2)cc1 ; c1cc(-c2ccc(N(c3ccc(-c4ccc5ccccc5c4)cc3)c3cccc4sc5ccccc5c34)cc2)cc(-c2cc3ccccc3c3ccccc23)c1]

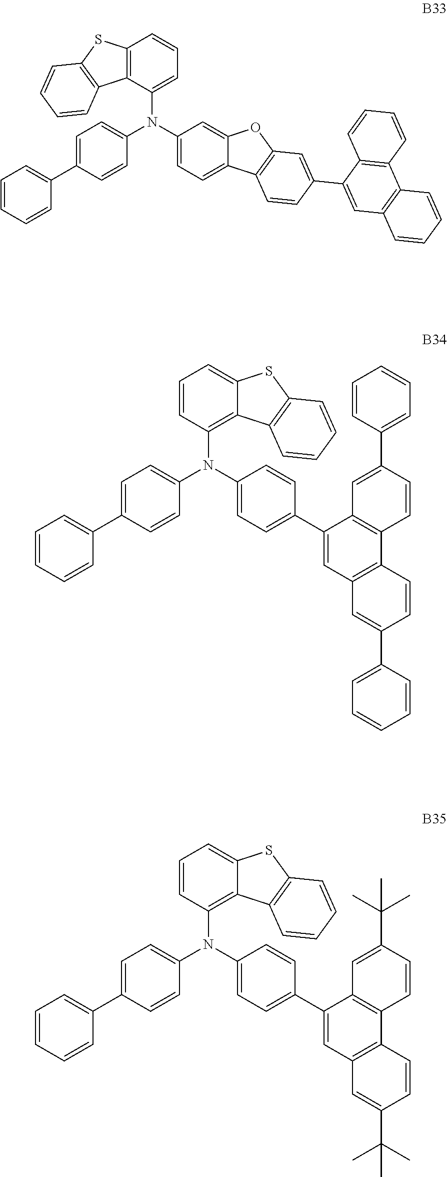 [SMILES: c1ccc(-c2ccc(N(c3ccc4c(c3)oc3cc(-c5cc6ccccc6c6ccccc56)ccc34)c3cccc4sc5ccccc5c34)cc2)cc1 ; c1ccc(-c2ccc(N(c3ccc(-c4cc5cc(-c6ccccc6)ccc5c5ccc(-c6ccccc6)cc45)cc3)c3cccc4sc5ccccc5c34)cc2)cc1 ; CC(C)(C)c1ccc2c(c1)cc(-c1ccc(N(c3ccc(-c4ccccc4)cc3)c3cccc4sc5ccccc5c34)cc1)c1cc(C(C)(C)C)ccc12]

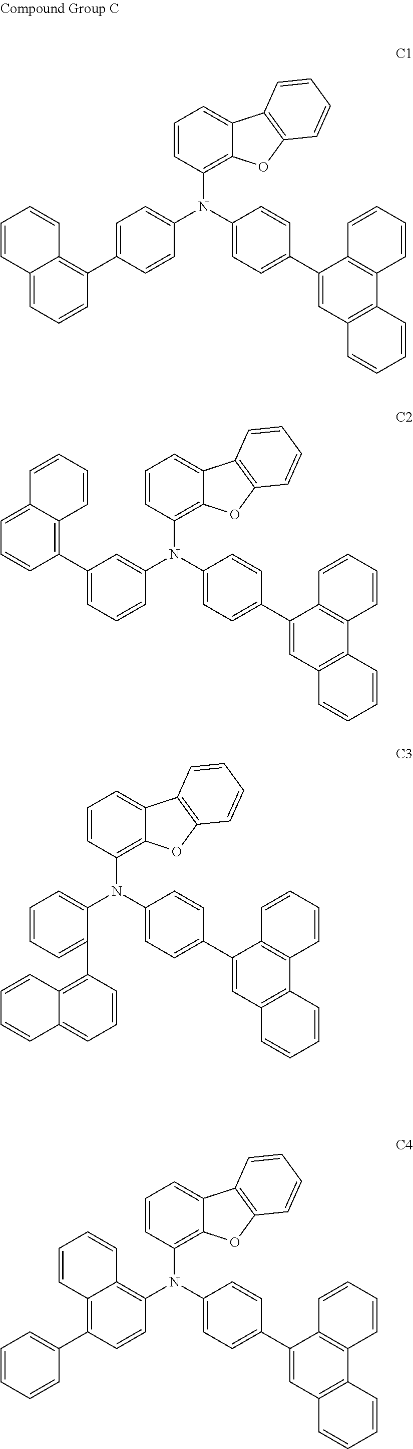 [SMILES: c1ccc2c(-c3ccc(N(c4ccc(-c5cc6ccccc6c6ccccc56)cc4)c4cccc5c4oc4ccccc45)cc3)cccc2c1 ; c1cc(-c2cccc3ccccc23)cc(N(c2ccc(-c3cc4ccccc4c4ccccc34)cc2)c2cccc3c2oc2ccccc23)c1 ; c1ccc(N(c2ccc(-c3cc4ccccc4c4ccccc34)cc2)c2cccc3c2oc2ccccc23)c(-c2cccc3ccccc23)c1 ; c1ccc(-c2ccc(N(c3ccc(-c4cc5ccccc5c5ccccc45)cc3)c3cccc4c3oc3ccccc34)c3ccccc23)cc1]

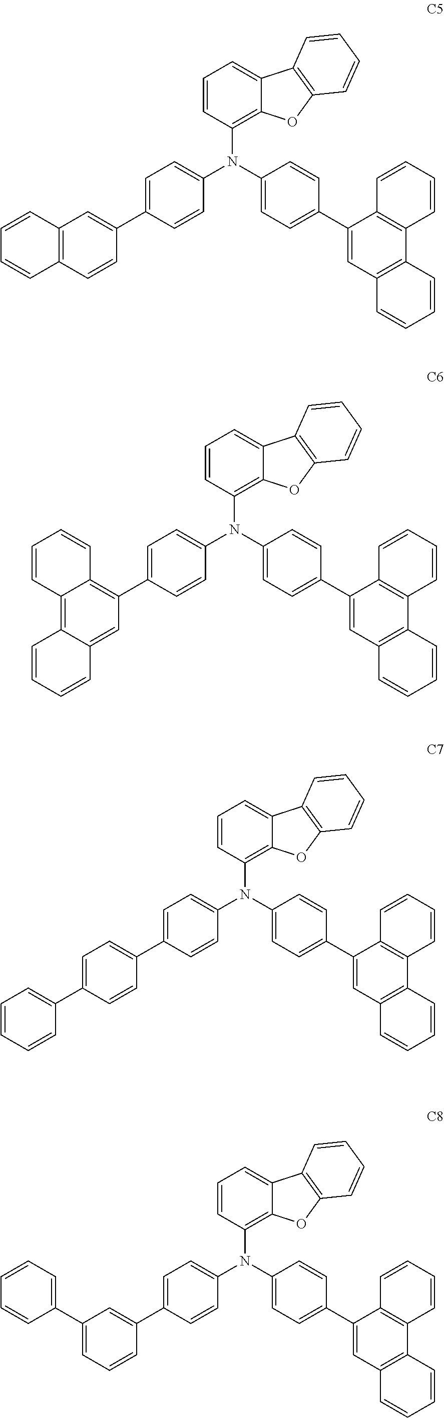 [SMILES: c1ccc2cc(-c3ccc(N(c4ccc(-c5cc6ccccc6c6ccccc56)cc4)c4cccc5c4oc4ccccc45)cc3)ccc2c1 ; c1ccc2c(c1)cc(-c1ccc(N(c3ccc(-c4cc5ccccc5c5ccccc45)cc3)c3cccc4c3oc3ccccc34)cc1)c1ccccc12 ; c1ccc(-c2ccc(-c3ccc(N(c4ccc(-c5cc6ccccc6c6ccccc56)cc4)c4cccc5c4oc4ccccc45)cc3)cc2)cc1 ; c1ccc(-c2cccc(-c3ccc(N(c4ccc(-c5cc6ccccc6c6ccccc56)cc4)c4cccc5c4oc4ccccc45)cc3)c2)cc1]

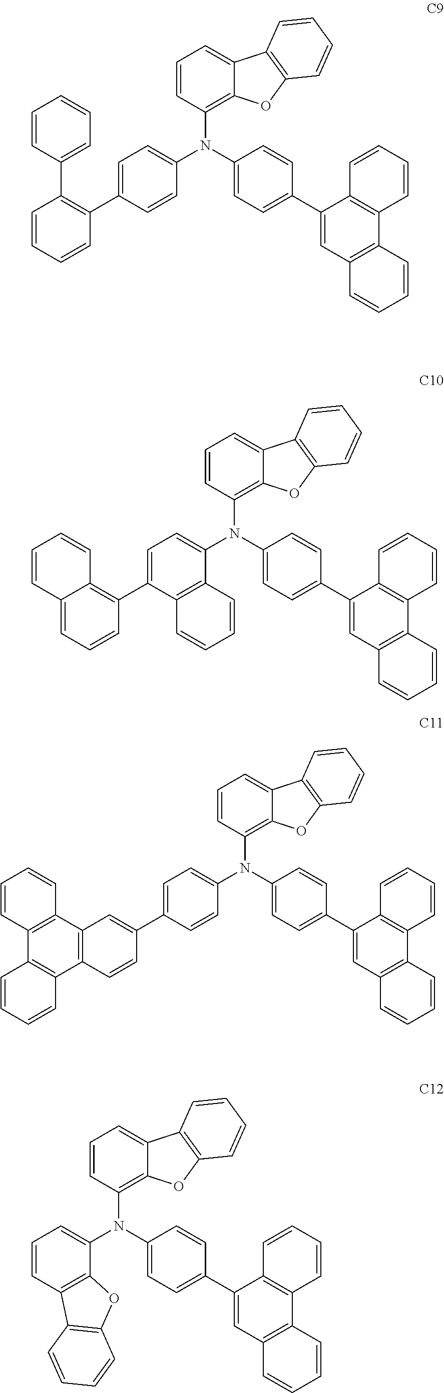 [SMILES: c1ccc(-c2ccccc2-c2ccc(N(c3ccc(-c4cc5ccccc5c5ccccc45)cc3)c3cccc4c3oc3ccccc34)cc2)cc1 ; c1ccc2c(-c3ccc(N(c4ccc(-c5cc6ccccc6c6ccccc56)cc4)c4cccc5c4oc4ccccc45)c4ccccc34)cccc2c1 ; c1ccc2c(c1)cc(-c1ccc(N(c3ccc(-c4ccc5c6ccccc6c6ccccc6c5c4)cc3)c3cccc4c3oc3ccccc34)cc1)c1ccccc12 ; c1ccc2c(c1)cc(-c1ccc(N(c3cccc4c3oc3ccccc34)c3cccc4c3oc3ccccc34)cc1)c1ccccc12]

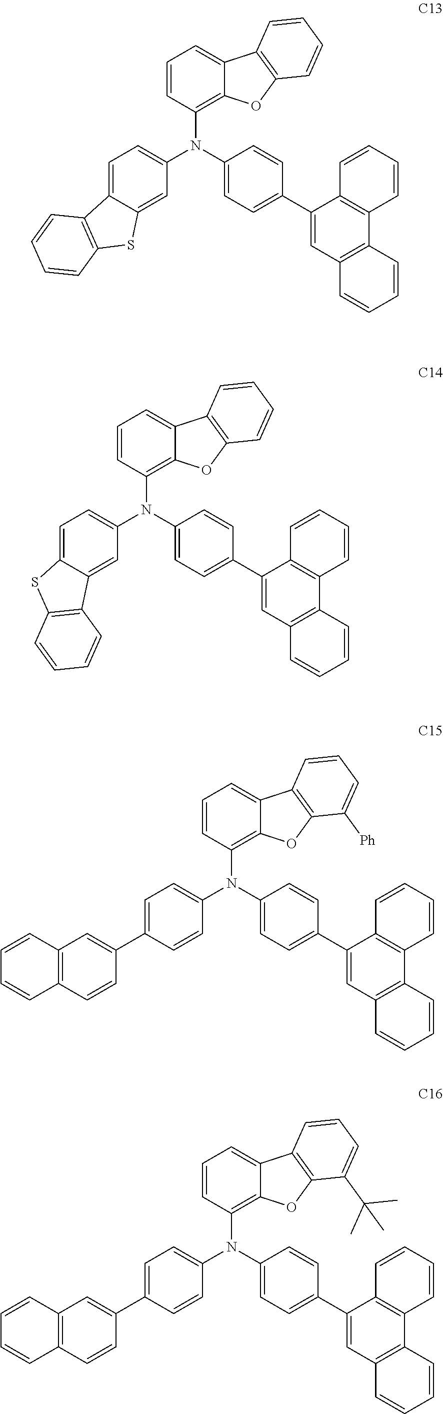 [SMILES: c1ccc2c(c1)cc(-c1ccc(N(c3ccc4c(c3)sc3ccccc34)c3cccc4c3oc3ccccc34)cc1)c1ccccc12 ; c1ccc2c(c1)cc(-c1ccc(N(c3ccc4sc5ccccc5c4c3)c3cccc4c3oc3ccccc34)cc1)c1ccccc12 ; c1ccc(-c2cccc3c2oc2c(N(c4ccc(-c5ccc6ccccc6c5)cc4)c4ccc(-c5cc6ccccc6c6ccccc56)cc4)cccc23)cc1 ; CC(C)(C)c1cccc2c1oc1c(N(c3ccc(-c4ccc5ccccc5c4)cc3)c3ccc(-c4cc5ccccc5c5ccccc45)cc3)cccc12]

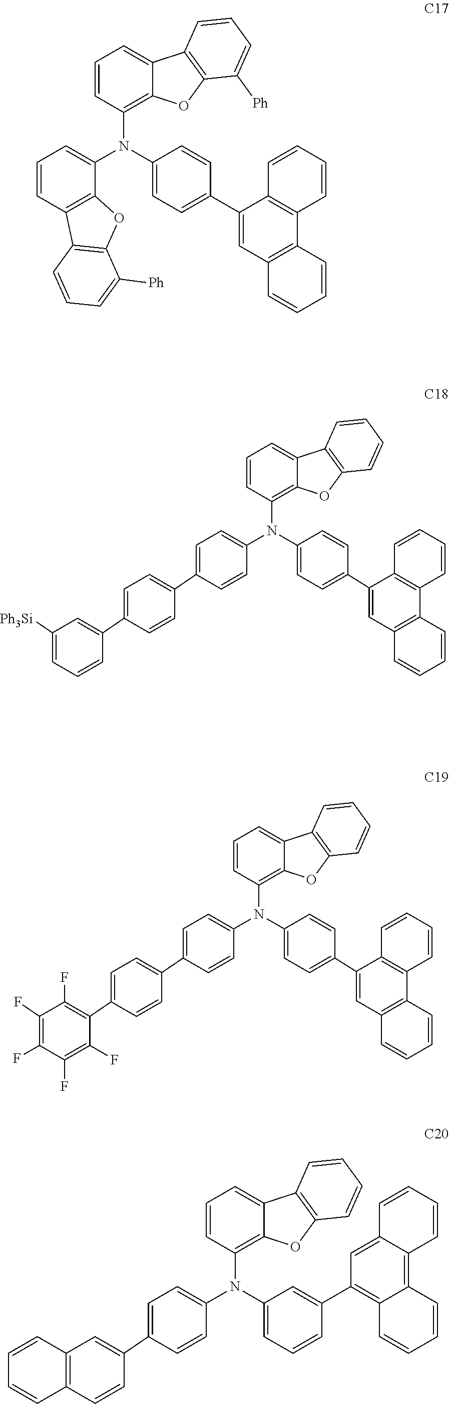

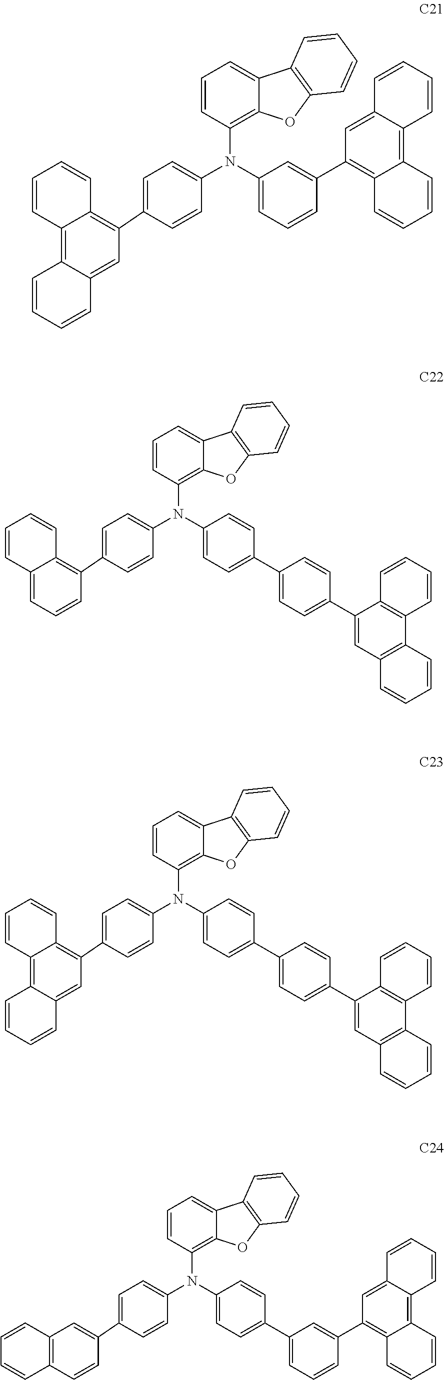 [SMILES: c1cc(-c2cc3ccccc3c3ccccc23)cc(N(c2ccc(-c3cc4ccccc4c4ccccc34)cc2)c2cccc3c2oc2ccccc23)c1 ; c1ccc2c(-c3ccc(N(c4ccc(-c5ccc(-c6cc7ccccc7c7ccccc67)cc5)cc4)c4cccc5c4oc4ccccc45)cc3)cccc2c1 ; c1ccc2c(c1)cc(-c1ccc(-c3ccc(N(c4ccc(-c5cc6ccccc6c6ccccc56)cc4)c4cccc5c4oc4ccccc45)cc3)cc1)c1ccccc12 ; c1cc(-c2ccc(N(c3ccc(-c4ccc5ccccc5c4)cc3)c3cccc4c3oc3ccccc34)cc2)cc(-c2cc3ccccc3c3ccccc23)c1]

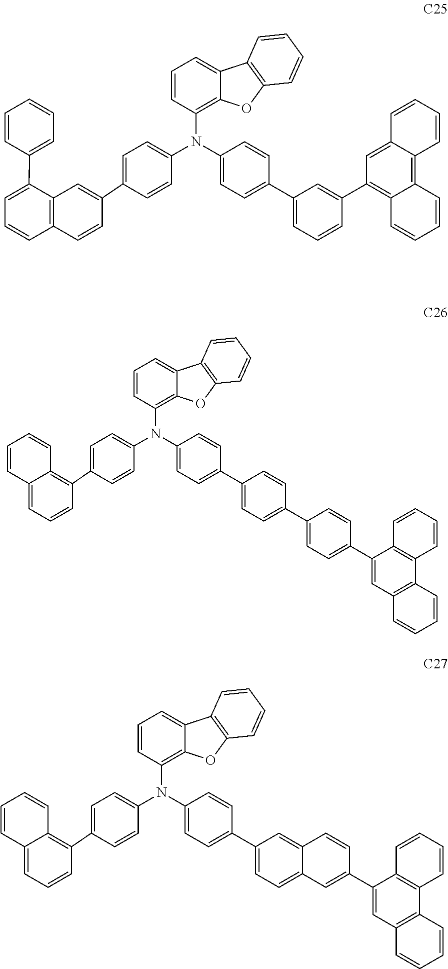 [SMILES: c1ccc(-c2cccc3ccc(-c4ccc(N(c5ccc(-c6cccc(-c7cc8ccccc8c8ccccc78)c6)cc5)c5cccc6c5oc5ccccc56)cc4)cc23)cc1 ; c1ccc2c(-c3ccc(N(c4ccc(-c5ccc(-c6ccc(-c7cc8ccccc8c8ccccc78)cc6)cc5)cc4)c4cccc5c4oc4ccccc45)cc3)cccc2c1 ; c1ccc2c(-c3ccc(N(c4ccc(-c5ccc6cc(-c7cc8ccccc8c8ccccc78)ccc6c5)cc4)c4cccc5c4oc4ccccc45)cc3)cccc2c1]

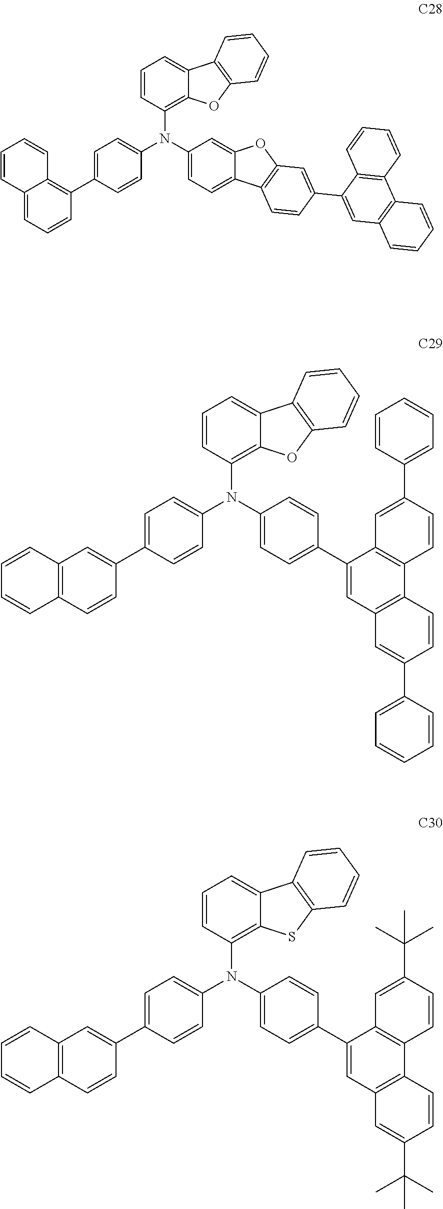 [SMILES: c1ccc2c(-c3ccc(N(c4ccc5c(c4)oc4cc(-c6cc7ccccc7c7ccccc67)ccc45)c4cccc5c4oc4ccccc45)cc3)cccc2c1 ; c1ccc(-c2ccc3c(c2)cc(-c2ccc(N(c4ccc(-c5ccc6ccccc6c5)cc4)c4cccc5c4oc4ccccc45)cc2)c2cc(-c4ccccc4)ccc23)cc1 ; CC(C)(C)c1ccc2c(c1)cc(-c1ccc(N(c3ccc(-c4ccc5ccccc5c4)cc3)c3cccc4c3sc3ccccc34)cc1)c1cc(C(C)(C)C)ccc12]

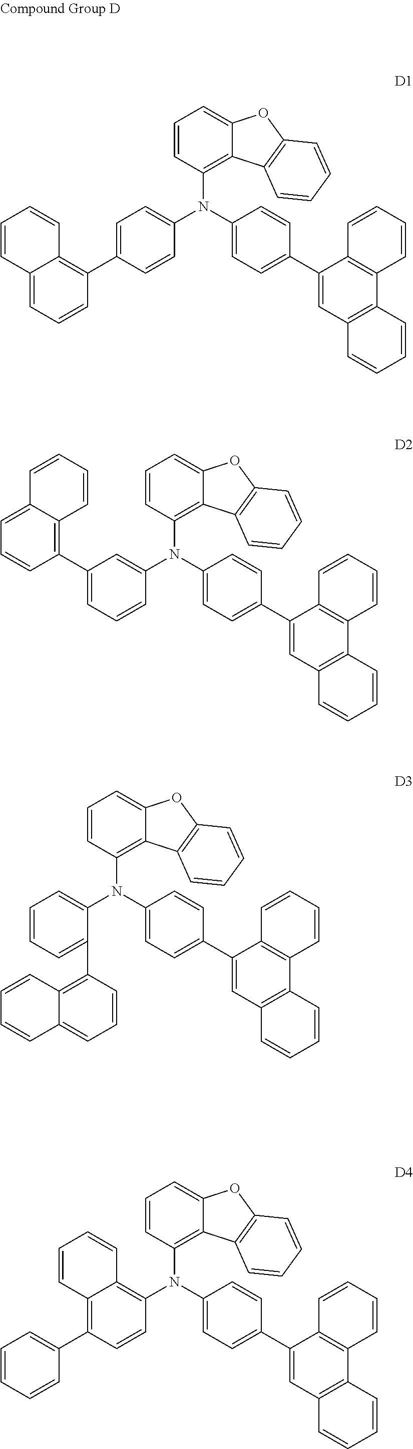 [SMILES: c1ccc2c(-c3ccc(N(c4ccc(-c5cc6ccccc6c6ccccc56)cc4)c4cccc5oc6ccccc6c45)cc3)cccc2c1 ; c1cc(-c2cccc3ccccc23)cc(N(c2ccc(-c3cc4ccccc4c4ccccc34)cc2)c2cccc3oc4ccccc4c23)c1 ; c1ccc(N(c2ccc(-c3cc4ccccc4c4ccccc34)cc2)c2cccc3oc4ccccc4c23)c(-c2cccc3ccccc23)c1 ; c1ccc(-c2ccc(N(c3ccc(-c4cc5ccccc5c5ccccc45)cc3)c3cccc4oc5ccccc5c34)c3ccccc23)cc1]

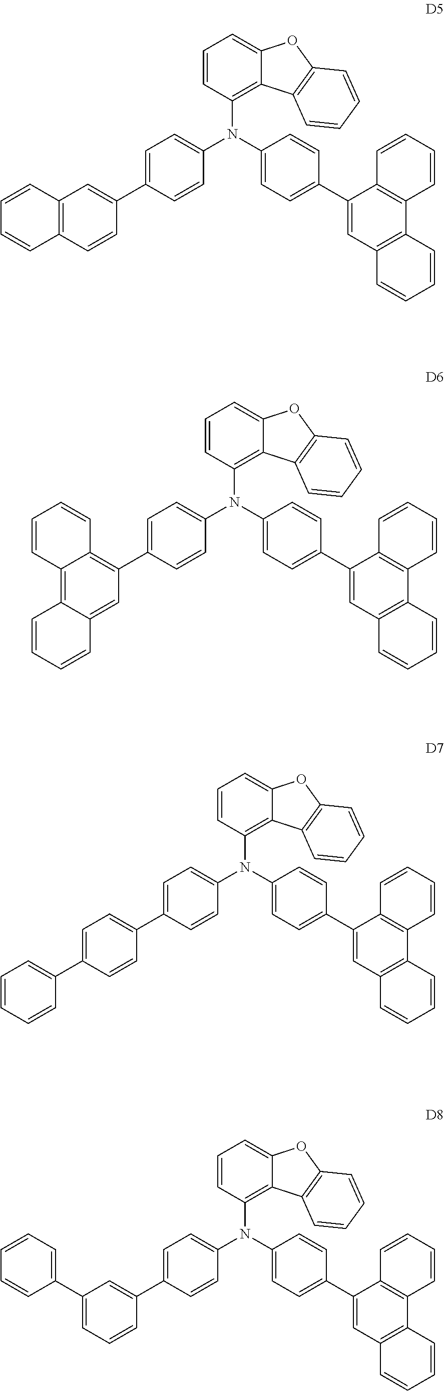 [SMILES: c1ccc2cc(-c3ccc(N(c4ccc(-c5cc6ccccc6c6ccccc56)cc4)c4cccc5oc6ccccc6c45)cc3)ccc2c1 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc(-c2ccc(-c3ccc(N(c4ccc(-c5cc6ccccc6c6ccccc56)cc4)c4cccc5oc6ccccc6c45)cc3)cc2)cc1 ; c1ccc(-c2cccc(-c3ccc(N(c4ccc(-c5cc6ccccc6c6ccccc56)cc4)c4cccc5oc6ccccc6c45)cc3)c2)cc1]

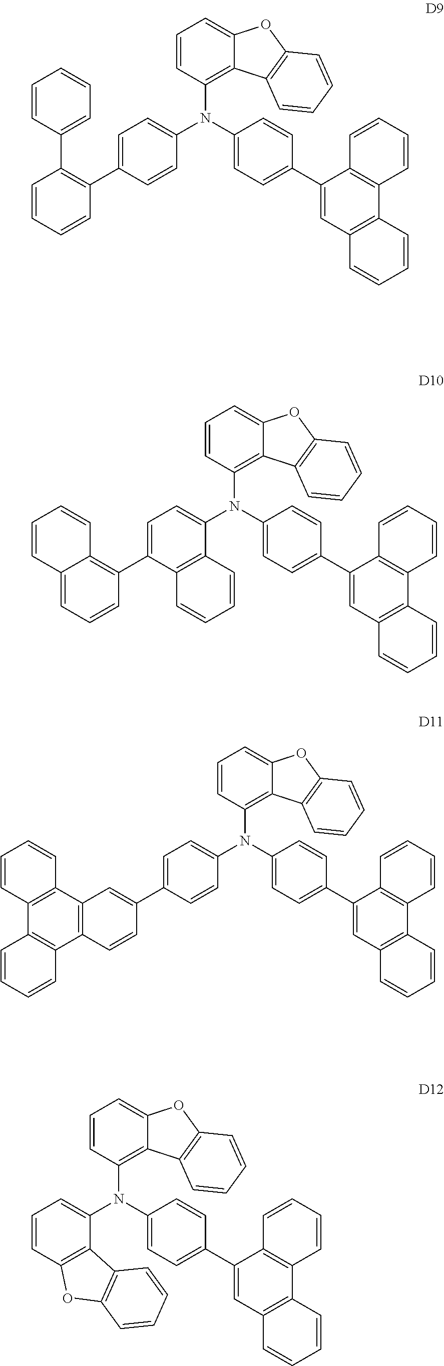 [SMILES: c1ccc(-c2ccccc2-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)c4ccccc34)cccc2c1 ; c1ccc2c(c1)cc(-c1ccc(N(c3ccc(-c4ccc5c6ccccc6c6ccccc6c5c4)cc3)c3cccc4oc5ccccc5c34)cc1)c1ccccc12 ; c1ccc2c(c1)cc(-c1ccc(N(c3cccc4oc5ccccc5c34)c3cccc4oc5ccccc5c34)cc1)c1ccccc12]

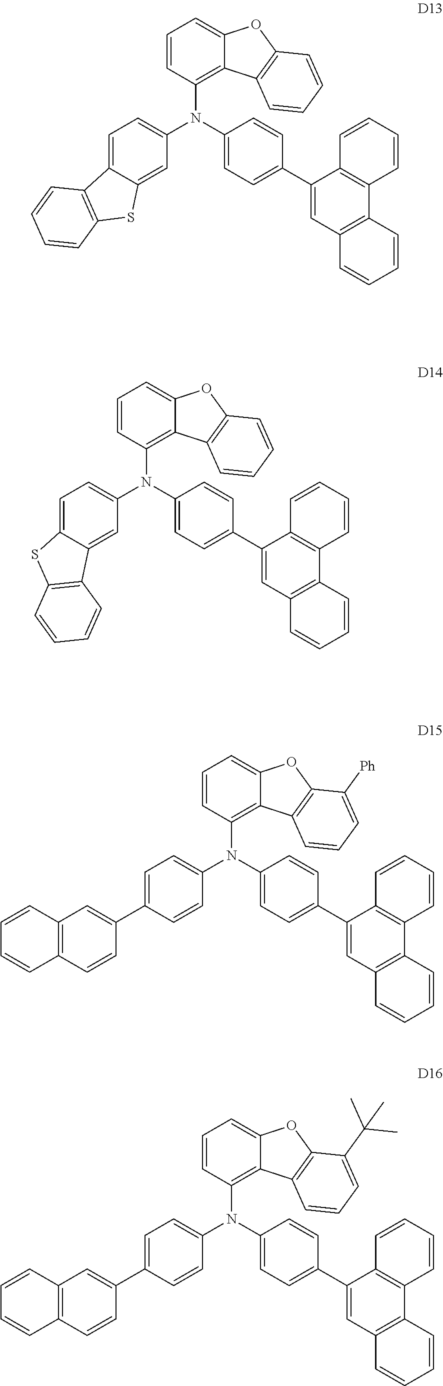 [SMILES: c1ccc2c(c1)cc(-c1ccc(N(c3ccc4c(c3)sc3ccccc34)c3cccc4oc5ccccc5c34)cc1)c1ccccc12 ; c1ccc2c(c1)cc(-c1ccc(N(c3ccc4sc5ccccc5c4c3)c3cccc4oc5ccccc5c34)cc1)c1ccccc12 ; c1ccc(-c2cccc3c2oc2cccc(N(c4ccc(-c5ccc6ccccc6c5)cc4)c4ccc(-c5cc6ccccc6c6ccccc56)cc4)c23)cc1 ; CC(C)(C)c1cccc2c1oc1cccc(N(c3ccc(-c4ccc5ccccc5c4)cc3)c3ccc(-c4cc5ccccc5c5ccccc45)cc3)c12]

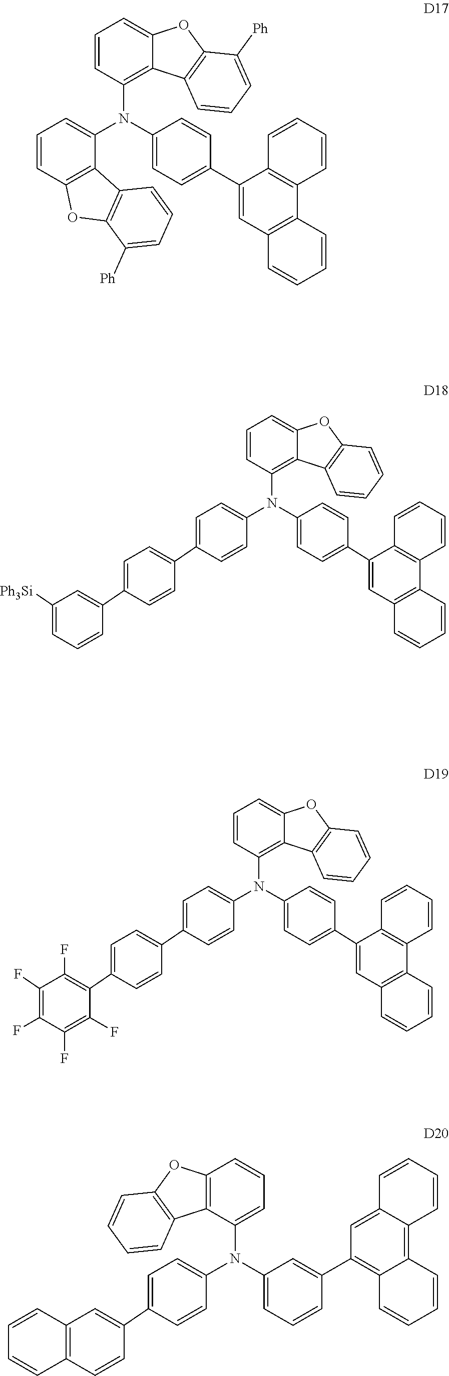

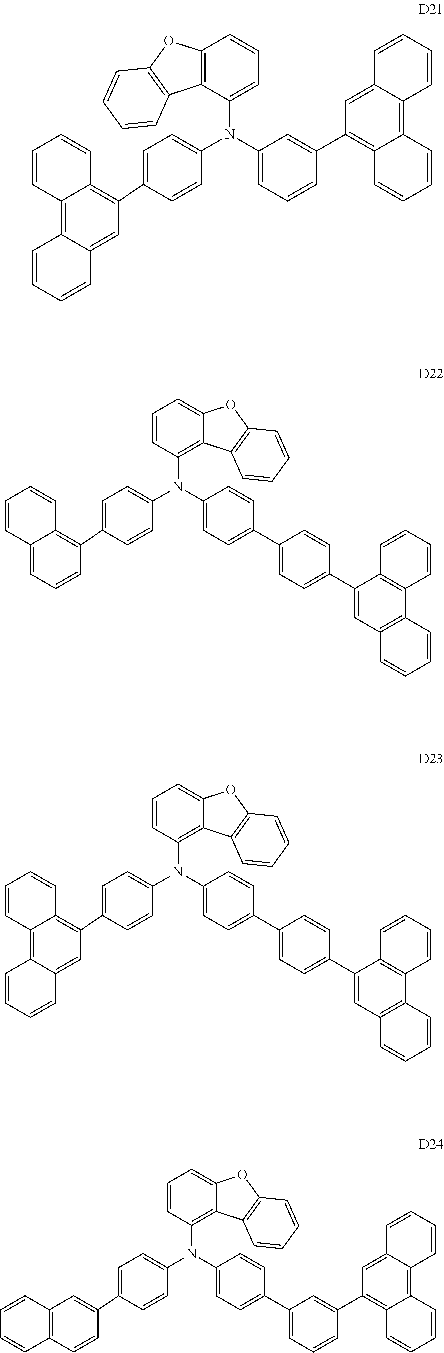 [SMILES: c1cc(-c2cc3ccccc3c3ccccc23)cc(N(c2ccc(-c3cc4ccccc4c4ccccc34)cc2)c2cccc3oc4ccccc4c23)c1 ; c1ccc2c(-c3ccc(N(c4ccc(-c5ccc(-c6cc7ccccc7c7ccccc67)cc5)cc4)c4cccc5oc6ccccc6c45)cc3)cccc2c1 ; c1ccc2c(c1)cc(-c1ccc(-c3ccc(N(c4ccc(-c5cc6ccccc6c6ccccc56)cc4)c4cccc5oc6ccccc6c45)cc3)cc1)c1ccccc12 ; c1cc(-c2ccc(N(c3ccc(-c4ccc5ccccc5c4)cc3)c3cccc4oc5ccccc5c34)cc2)cc(-c2cc3ccccc3c3ccccc23)c1]

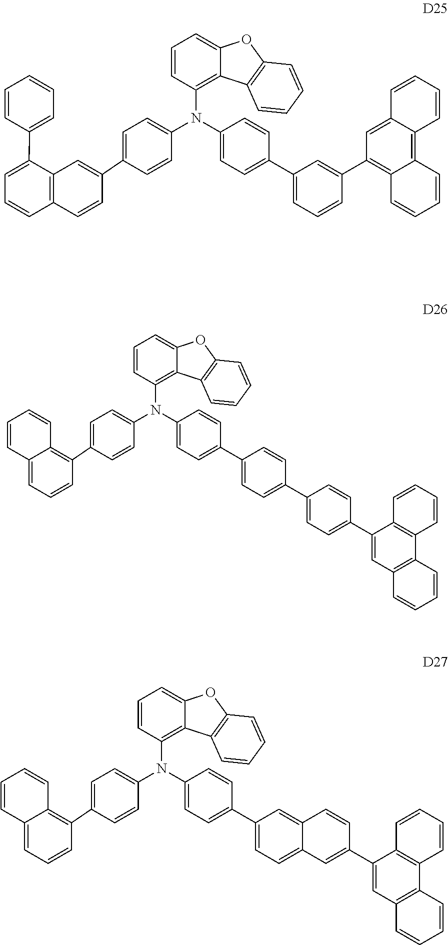 [SMILES: c1ccc(-c2cccc3ccc(-c4ccc(N(c5ccc(-c6cccc(-c7cc8ccccc8c8ccccc78)c6)cc5)c5cccc6oc7ccccc7c56)cc4)cc23)cc1 ; c1ccc2c(-c3ccc(N(c4ccc(-c5ccc(-c6ccc(-c7cc8ccccc8c8ccccc78)cc6)cc5)cc4)c4cccc5oc6ccccc6c45)cc3)cccc2c1 ; c1ccc2c(-c3ccc(N(c4ccc(-c5ccc6cc(-c7cc8ccccc8c8ccccc78)ccc6c5)cc4)c4cccc5oc6ccccc6c45)cc3)cccc2c1]

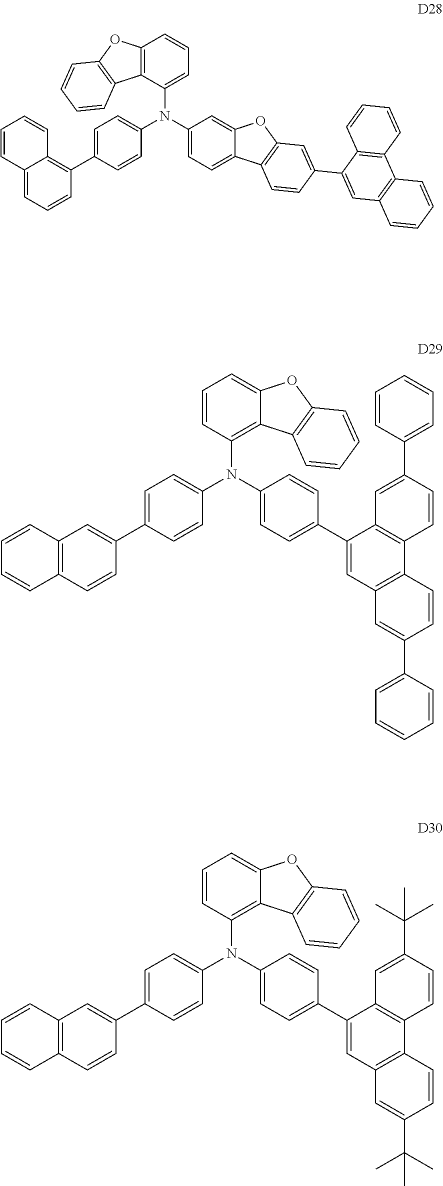 [SMILES: c1ccc2c(-c3ccc(N(c4ccc5c(c4)oc4cc(-c6cc7ccccc7c7ccccc67)ccc45)c4cccc5oc6ccccc6c45)cc3)cccc2c1 ; c1ccc(-c2ccc3c(c2)cc(-c2ccc(N(c4ccc(-c5ccc6ccccc6c5)cc4)c4cccc5oc6ccccc6c45)cc2)c2cc(-c4ccccc4)ccc23)cc1 ; CC(C)(C)c1ccc2c(c1)cc(-c1ccc(N(c3ccc(-c4ccc5ccccc5c4)cc3)c3cccc4oc5ccccc5c34)cc1)c1cc(C(C)(C)C)ccc12]

The hole injection layer HIL may include, for example, a phthalocyanine compound such as copper phthalocyanine, N,N′-diphenyl-N,N-bis-[4-(phenyl-m-tolyl-amino)-phenyl]-biphenyl-4,4′-diamine (DNTPD), 4,4′,4″-tris(3-methylphenylphenylamino) triphenylamine (m-MTDATA), 4,4′,4″-tris(N,N-diphenylamino)triphenylamine (TDATA), 4,4′,4″-tris{N,-2-naphthyl-N-(phenyl)amino}-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium tetrakis(pentafluorophenyl)borate, and dipyrazino[2,3-f:2′,3′-h]quinoxaline-2,3,6,7,10,11-hexacarbonitrile (HAT-CN).

The hole transport layer HTL may include, for example, carbazole derivatives such as N-phenyl carbazole and polyvinyl carbazole, fluorene-based derivatives, N,N′-bis(3-methylphenyl)-N,N′-diphenyl-[1,1-biphenyl]-4,4′-diamine (TPD), triphenylamine-based derivatives such as 4,4′,4″-tris(carbazol-9-yl)triphenylamine (TCTA), N,N′-di(naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), 9-(4-tert-butylphenyl)-3,6-bis(triphenylsilyl)-9H-carbazole (CzSi), etc.

The thickness of the hole transport region HTR may be from about 100 Å to about 10,000 Å, for example, from about 100 Å to about 5,000 Å. The thickness of the hole injection region HIL may be, for example, from about 30 Å to about 1,000 Å, and the thickness of the hole transport layer HTL may be from about 30 Å to about 1,000 Å. For example, the thickness of the electron blocking layer EBL may be from about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be achieved without substantial increase of a driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may be one of quinone derivatives, metal oxides, or cyano group-containing compounds, without limitation. For example, non-limiting examples of the p-dopant may include one or more quinone derivatives such as tetracyanoquinodimethane (TCNQ) and 2,3,5,6-tetrafluoro-7,7,8,8-tetracyanoquinodimethane (F4-TCNQ), one or more metal oxides such as tungsten oxide and molybdenum oxide, etc., without limitation.

As described above, the hole transport region HTR may further include at least one of a hole buffer layer or an electron blocking layer EBL in addition to the hole injection layer HIL and the hole transport layer HTL. The hole buffer layer may compensate an optical resonance distance according to the wavelength of light emitted from an emission layer EML to increase light emission efficiency. Materials which may be included in the hole transport region HTR may be used as materials included in the hole buffer layer. The electron blocking layer EBL is a layer playing the role of preventing the electron injection from the electron transport region ETR to the hole transport region HTR.

The emission layer EML is disposed on the hole transport region HTR. The thickness of the emission layer EML may be, for example, about 100 Å to about 1,000 Å or about 100 Å to about 300 Å. The emission layer EML may have a single layer formed using a single material, a single layer formed using a plurality of different materials, or a multilayer structure having a plurality of layers formed using a plurality of different materials.

In the luminescence device 10 of some exemplary embodiments, the emission layer EML may include one or more anthracene derivatives, pyrene derivatives, fluoranthene derivatives, chrysene derivatives, dihydrobenzanthracene derivatives, or triphenylene derivatives. Particularly, the emission layer EML may include one or more anthracene derivatives or pyrene derivatives.

The emission layer EML may include an anthracene derivative represented by the following Formula A:

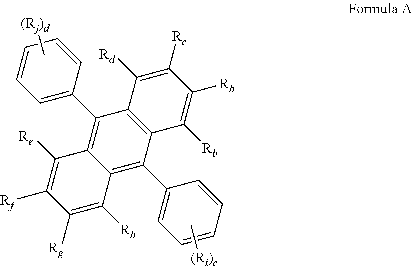

In Formula A, Rₐ to Rⱼ may be each independently a hydrogen atom, a deuterium atom, a halogen atom, a substituted or unsubstituted silyl group, a substituted or unsubstituted alkyl group of 1 to 10 carbon atoms, a substituted or unsubstituted aryl amine group of 6 to 30 carbon atoms for forming a ring, or a substituted or unsubstituted heteroaryl group of 2 to 30 carbon atoms for forming a ring, or combined with an adjacent group to form a ring. The variables Rₐ to Rⱼ may be combined with an adjacent group to form a saturated hydrocarbon ring or an unsaturated hydrocarbon ring.

In Formula A, “c” and “d” may be each independently an integer of 0 to 5.

If “c” is an integer of 2 or more, a plurality of Rᵢ groups may be the same or different. If “d” is an integer of 2 or more, a plurality of Rⱼ groups may be the same or different.

Formula A may be represented by any one among the following Formula 3-1 to Formula 3-16:

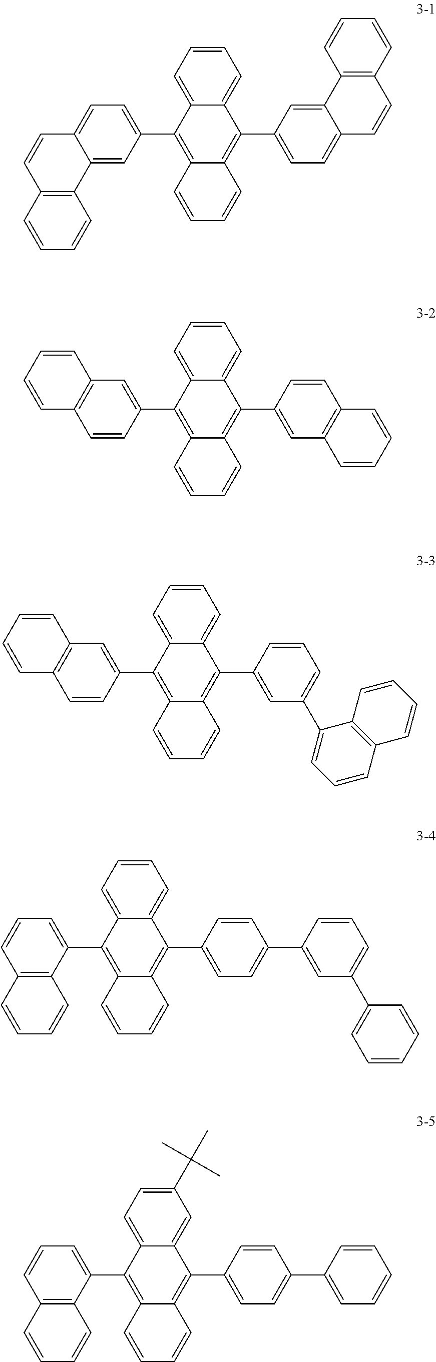

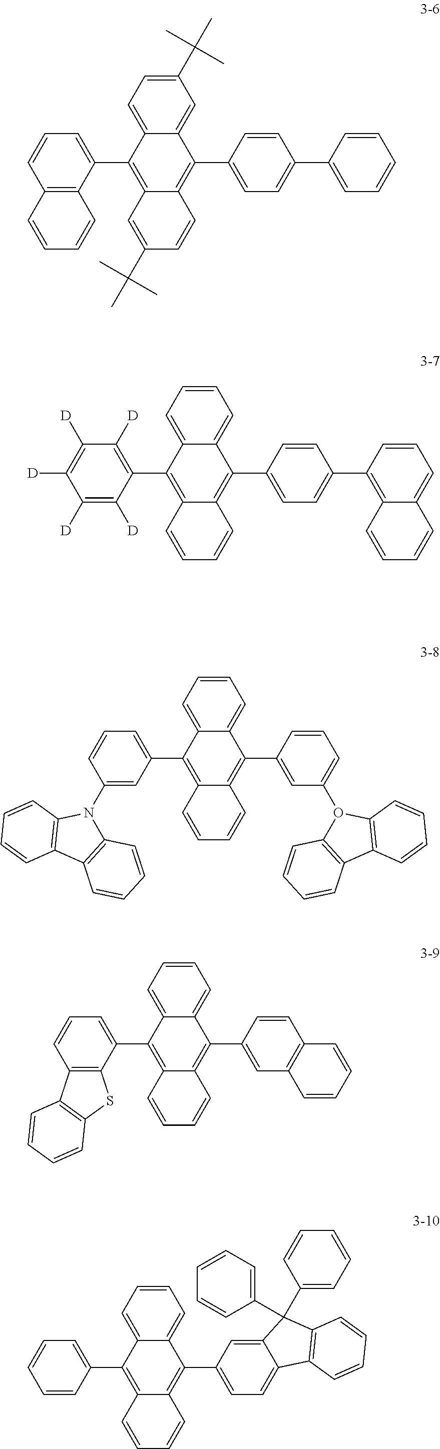

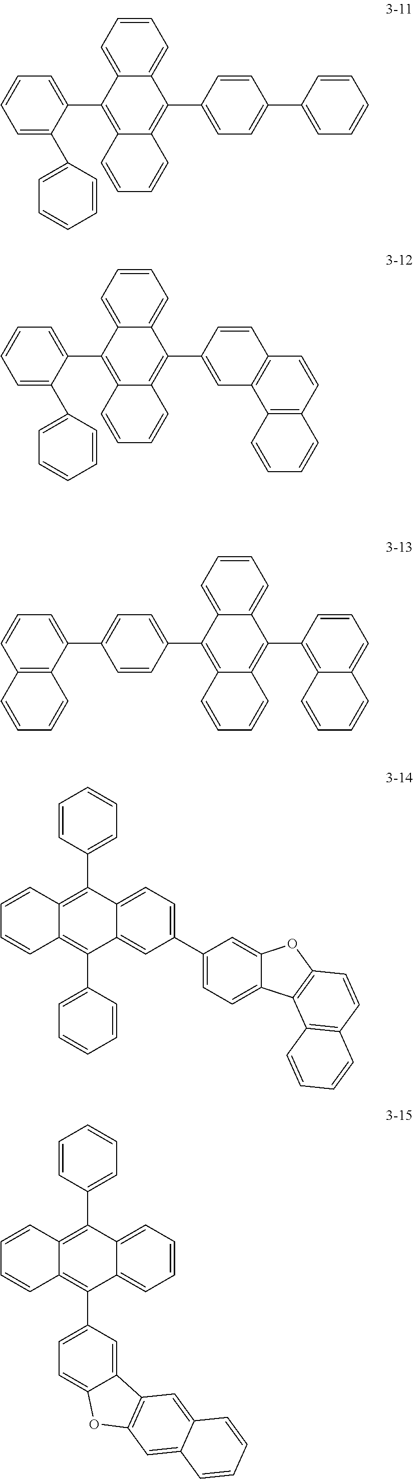

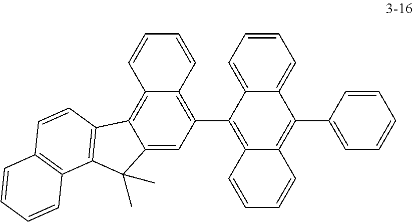 [SMILES: CC1(C)c2cc(-c3c4ccccc4c(-c4ccccc4)c4ccccc34)c3ccccc3c2-c2ccc3ccccc3c21]

In the luminescence devices 10 of some exemplary embodiments as shown in FIG. 1 to FIG. 4, the emission layer EML may include a host and a dopant, and the emission layer EML may include the compounds represented by the above-described chemical formulae as host materials.

The emission layer EML may further include commonly used materials well known as the host material in the art. For example, the emission layer EML may include as a host material, at least one of bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris (carbazol-9-yl) triphenylamine or 1,3,5-tris(1-phenyl-1H-benzo[d]imidazol-2-yl)benzene (TPBi). However, the exemplary embodiments are not limited thereto. For example, tris(8-hydroxyquinolino)aluminum (Alq₃), 3-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), 9-(4-tert-butylphenyl)-3,6-bis(triphenylsilyl)-9H-carbazole (CzSi), etc. may be used as the host material.

In some exemplary embodiments, the emission layer EML may include as the dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, 1,4-bis(N,N-diphenylamino)pyrene), etc.

In some exemplary embodiments, the emission layer EML may emit blue light or green light. The emission layer EML may emit blue light having a central wavelength of from about 390 nm to less than about 500 nm, or green light having a central wavelength of from about 500 nm to about 600 nm. However, the exemplary embodiments are not limited thereto, and the emission layer EML may emit light other than blue light and green light. The emission layer EML may emit fluorescence, delayed fluorescence, or phosphorescence.

In the luminescence devices 10 of exemplary embodiments as shown in FIGS. 1 to 4, the electron transport region ETR is disposed on the emission layer EML. The electron transport region ETR may include at least one of a hole blocking layer HBL, an electron transport layer ETL or an electron injection layer EIL. However, the exemplary embodiments are not limited thereto.

The electron transport region ETR may have a single layer formed using a single material, a single layer formed using a plurality of different materials, or a multilayer structure having a plurality of layers formed using a plurality of different materials.

For example, the electron transport region ETR may have a single layer structure of an electron injection layer EIL or an electron transport layer ETL, or a single layer structure formed using an electron injection material and an electron transport material. Also, the electron transport region ETR may have a single layer structure formed using a plurality of different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

If the electron transport region ETR includes an electron transport layer ETL, the electron transport region ETR may include an anthracene-based compound. The exemplary embodiments are not limited thereto. For example, the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq2), 9,10-di(naphthalene-2-yl)anthracene (ADN), 1,3-bis[3,5-di(pyridine-3-yl)phenyl]benzene (BmPyPhB), or a mixture thereof. The thickness of the electron transport layer ETL may be from about 100 Å to about 1,000 Å and may be, for example, from about 150 Å to about 500 Å. If the thickness of the electron transport layer ETL satisfies the above-described range, satisfactory electron transport properties may be obtained without substantial increase of a driving voltage.

If the electron transport region ETR includes the electron injection layer EIL, the electron transport region ETR may use a metal halide such as LiF, NaCl, CsF, RbCl, RbI, and CuI, a metal in lanthanoides such as Yb, a metal oxide such as Li₂O and BaO, or lithium quinolate (LiQ). However, the exemplary embodiments are not limited thereto. The electron injection layer EIL may also be formed using a mixture material of an electron transport material and an insulating organo metal salt. The organo metal salt may be a material having an energy band gap of about 4 eV or more. Particularly, the organo metal salt may include, for example, one or more metal acetates, metal benzoates, metal acetoacetates, metal acetylacetonates, or metal stearates. The thickness of the electron injection layer EIL may be from about 1 Å to about 100 Å, and from about 3 Å to about 90 Å. If the thickness of the electron injection layer EIL satisfies the above described range, satisfactory electron injection properties may be obtained without inducing substantial increase of a driving voltage.

The electron transport region ETR may include a hole blocking layer HBL as described above. The hole blocking layer HBL may include, for example, at least one of 2,9-dimethyl-4,7-diphenyl-1,10-phenanthroline (BCP), or 4,7-diphenyl-1,10-phenanthroline (Bphen). However, the exemplary embodiments are not limited thereto.

The second electrode EL2 is disposed on the electron transport region ETR. The second electrode EL2 may be a common electrode or a cathode. The second electrode EL2 may be a transmissive electrode, a transflective electrode or a reflective electrode. If the second electrode EL2 is the transmissive electrode, the second electrode EL2 may be formed using at least one transparent metal oxide, for example, ITO, IZO, ZnO, and ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). Alternatively, the second electrode EL2 may have a multilayered structure including a reflective layer or a transflective layer formed using the above-described materials and a transparent conductive layer formed using an ITO, an IZO, a ZnO, an ITZO, etc.

The second electrode EL2 may be connected with an auxiliary electrode. If the second electrode EL2 is connected with the auxiliary electrode, the resistance of the second electrode EL2 may decrease.

On the second electrode EL2 of the luminescence device 10, a capping layer (CPL) may be further disposed. In some exemplary embodiments. The capping layer CPL may include, for example, 2,2′-Dimethyl-N,N′-di-[(1-naphthyl)-N,N′-diphenyl]-1,1′-biphenyl-4,4′-diamine (α-NPD), NPB, TPD, m-MTDATA, Alq₃, copper(II) phthalocyanine (CuPc), N4,N4,N4′,N4′-tetra(biphenyl-4-yl) biphenyl-4,4′-diamine (TPD15), 4,4′,4″-tris(carbazol-9-yl)triphenylamine (TCTA), N,N′-bis(naphthalen-1-yl), etc.

The above-described compound may be included in an organic layer other than the hole transport region HTR as a material for the luminescence device 10. The luminescence device 10 according to some exemplary embodiments may include the above-described compound in at least one organic layer disposed between the first electrode EL1 and the second electrode EL2, or in the capping layer (CPL) disposed on the second electrode EL2.

In the luminescence device 10, according to the application of voltages to the first electrode EL1 and second electrode EL2, respectively, holes injected from the first electrode EL1 may move via the hole transport region HTR to the emission layer EML, and electrons injected from the second electrode EL2 may move via the electron transport region ETR to the emission layer EML. The electrons and the holes are recombined in the emission layer EML to produce excitons, and the excitons may emit light via transition from an excited state to a ground state.

### 1. Synthetic Examples

The amine compound of some exemplary embodiments may be synthesized, for example, by the following. However, the synthetic method of making the amine compound of the exemplary embodiments is not limited thereto.

1-1. Synthesis of Compound A1

Compound A1 of an exemplary embodiment may be synthesized, for example, although not wanting to be bound by theory, by the following Reaction 1:

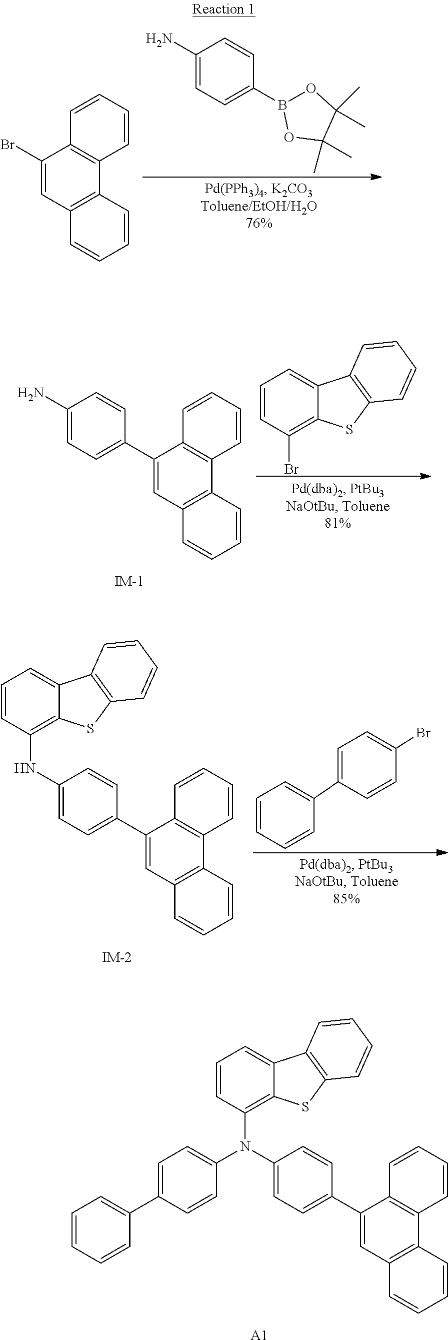

Synthesis of Intermediate IM-1

Under an argon atmosphere, to a 1,000 ml, three-neck flask, 20.00 g (77.8 mmol) of 9-bromophenanthrene, 20.45 g (1.2 weight equivalent, 93.4 mmol) of 4-(4,4,5,5-tetramethyl-1,3,2-dioxaborolan-2-yl)aniline, 32.25 g (3.0 weight equivalent, 233.3 mmol) of K₂CO₃, 4.49 g (0.05 weight equivalent, 3.9 mmol) of Pd(PPh₃)₄, and 545 ml of a mixture solution of toluene/ethanol/H₂O (a volumetric ratio of 4/2/1) were added in order, followed by heating and stirring at about 80° C. After cooling to room temperature in the air, the reaction solution was extracted with toluene. An aqueous layer was removed, and an organic layer was washed with a saturated saline solution and dried with an anhydrous magnesium sulfate. The anhydrous magnesium sulfate was strained, and an organic layer was concentrated. The crude product thus obtained was separated by silica gel column chromatography (using a mixture solvent of hexane and toluene as a developing layer) to obtain Intermediate IM-1 (15.92 g, yield 76%).

In order to identify Intermediate IM-1, fast atom bombardment mass spectrometry (FAB-MS) was measured using a device sold under the trade designation JMS-700V of JEOL Ltd. of Tokyo, Japan. From the measurement results of FAB-MS, mass number of m/z=269 was observed as a molecular ion peak and Intermediate IM-1 was identified.

Synthesis of Intermediate IM-2

Under an argon atmosphere, to a 300 ml, three-neck flask, 10.00 g (37.1 mmol) of Intermediate IM-1, 0.64 g (0.03 weight equivalent, 1.1 mmol) of bis(dibenzylideneacetone)palladium Pd(dba)₂, 3.57 g (1.0 weight equivalent, 37.1 mmol) of NaOtBu, 185 ml of toluene, 10.75 g (1.1 weight equivalent, 40.8 mmol) of 4-bromodibenzothiophene and 0.75 g (0.1 weight equivalent, 3.7 mmol) of tBu₃P were added in order, followed by heating, refluxing and stirring. After cooling to room temperature in the air, water was added to the reaction solvent, and an organic layer was separately taken. Toluene was added to an aqueous layer, and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-2 (13.58 g, yield 81%).

From the measurement results of FAB-MS, mass number of m/z=451 was observed as a molecular ion peak and Intermediate IM-2 was identified.

Synthesis of Compound A1

Under an argon atmosphere, to a 300 ml, three-neck flask, 10.00 g (22.1 mmol) of Intermediate IM-2, 0.38 g (0.03 weight equivalent, 0.7 mmol) of Pd(dba)₂, 4.26 g (2.0 weight equivalent, 44.3 mmol) of NaOtBu, 110 ml of toluene, 5.68 g (1.1 weight equivalent, 24.4 mmol) of 4-bromobiphenyl and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained, and an organic layer was concentrated. The crude product thus obtained was separated by silica gel column chromatography (using a mixture solvent of hexane and toluene as a developing layer) to obtain Compound A1 (11.36 g, yield 85%) as a solid.

From the measurement results of FAB-MS, mass number of m/z=603 was observed as a molecular ion peak and Compound A1 was identified.

1-2. Synthesis of Compound A7

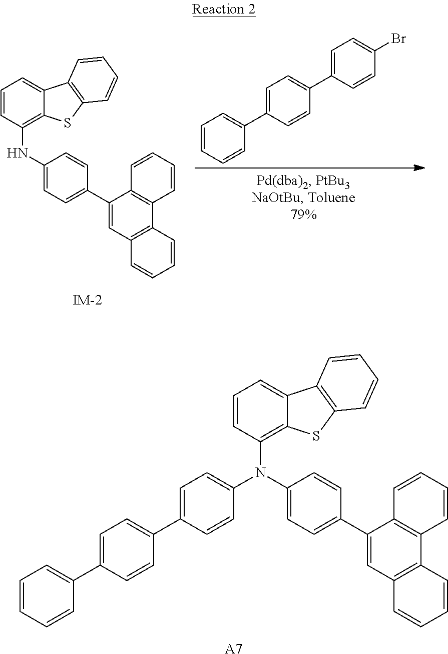

Synthesis of Compound A7

Under an argon atmosphere, to a 300 ml, three-neck flask, 10.00 g (22.1 mmol) of Intermediate IM-2, 0.38 g (0.03 weight equivalent, 0.7 mmol) of Pd(dba)₂, 4.26 g (2.0 weight equivalent, 44.3 mmol) of NaOtBu, 110 ml of toluene, 7.53 g (1.1 weight equivalent, 24.4 mmol) of 4-bromo-terphenyl and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer, and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound A7 (11.89 g, yield 79%) as a solid.

From the measurement results of FAB-MS, mass number of m/z=679 was observed as a molecular ion peak and Compound A7 was identified.

1-3 Synthesis of Compound A12

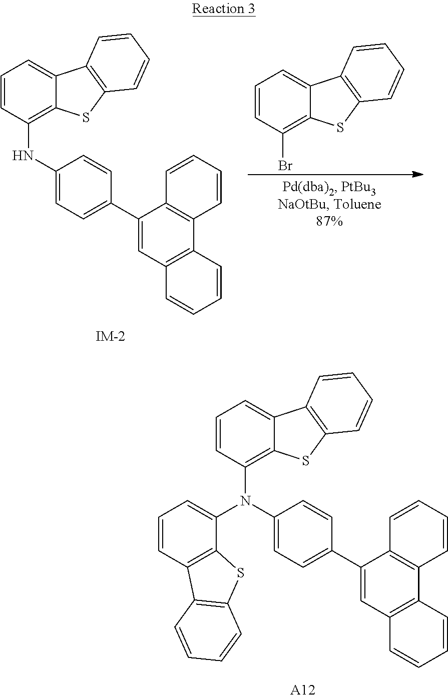

Synthesis of Compound A12

Under an argon atmosphere, to a 300 ml, three-neck flask, 10.00 g (22.1 mmol) of Intermediate IM-2, 0.38 g (0.03 weight equivalent, 0.7 mmol) of Pd(dba)₂, 4.26 g (2.0 weight equivalent, 44.3 mmol) of NaOtBu, 110 ml of toluene, 6.41 g (1.1 weight equivalent, 24.4 mmol) of 4-bromodibenzothiophene and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound A12 (12.21 g, yield 87%) as a solid.

From the measurement results of FAB-MS, mass number of m/z=633 was observed as a molecular ion peak and Compound A12 was identified.

1-4. Synthesis of Compound A18

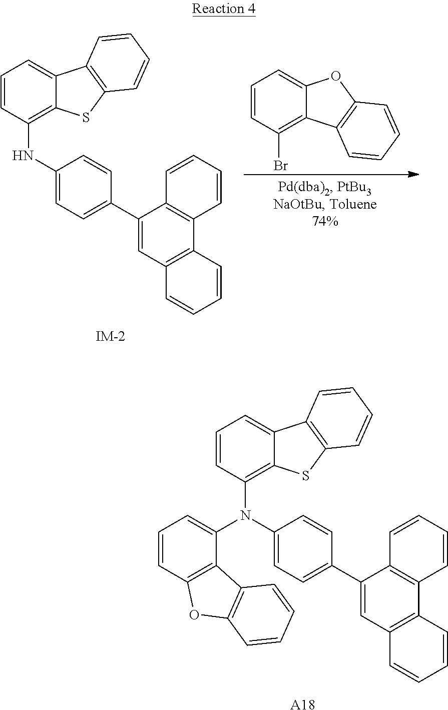

Synthesis of Compound A18

Under an argon atmosphere, to a 300 ml, three-neck flask, 10.00 g (22.1 mmol) of Intermediate IM-2, 0.38 g (0.03 weight equivalent, 0.7 mmol) of Pd(dba)₂, 4.26 g (2.0 weight equivalent, 44.3 mmol) of NaOtBu, 110 ml of toluene, 6.02 g (1.1 weight equivalent, 24.4 mmol) of 1-bromodibenzofuran and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound A18 (10.12 g, yield 74%) as a solid.

From the measurement results of FAB-MS, mass number of m/z=617 was observed as a molecular ion peak and Compound A18 was identified.

1-5. Synthesis of Compound B5

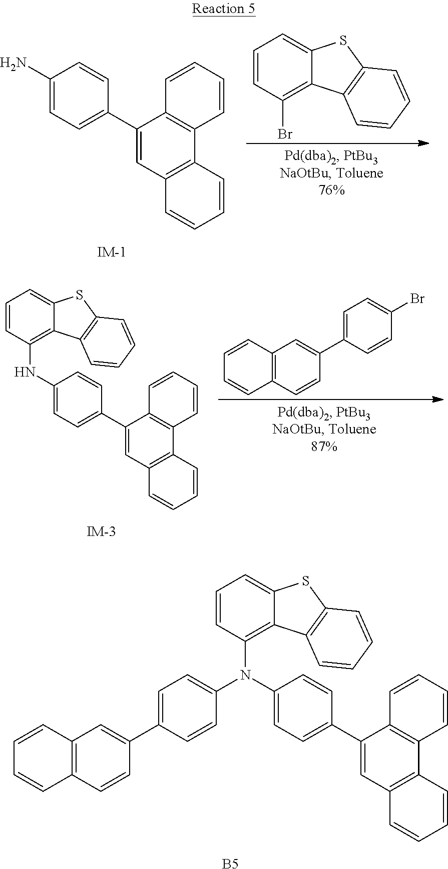

Synthesis of Intermediate IM-3

Under an argon atmosphere, to a 300 ml, three-neck flask, 10.00 g (37.1 mmol) of Intermediate IM-1, 0.64 g (0.03 weight equivalent, 1.1 mmol) of Pd(dba)₂, 3.57 g (1.0 weight equivalent, 37.1 mmol) of NaOtBu, 185 ml of toluene, 10.75 g (1.1 weight equivalent, 40.8 mmol) of 1-bromodibenzothiophene and 0.75 g (0.1 weight equivalent, 3.7 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-3 (12.74 g, yield 76%).

From the measurement results of FAB-MS, mass number of m/z=451 was observed as a molecular ion peak and Intermediate IM-3 was identified.

Synthesis of Compound B5

Under an argon atmosphere, to a 300 ml, three-neck flask, 10.00 g (22.1 mmol) of Intermediate IM-3, 0.38 g (0.03 weight equivalent, 0.7 mmol) of Pd(dba)₂, 4.26 g (2.0 weight equivalent, 44.3 mmol) of NaOtBu, 110 ml of toluene, 6.90 g (1.1 weight equivalent, 24.4 mmol) of 2-(4-bromophenyl)naphthalene and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound B5 (12.60 g, yield 87%) as a solid.

From the measurement results of FAB-MS, mass number of m/z=653 was observed as a molecular ion peak and Compound B5 was identified.

1-6. Synthesis of Compound B9

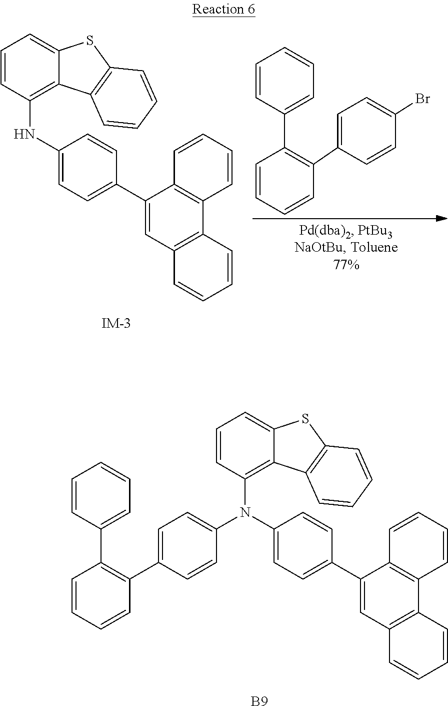

Synthesis of Compound B9

Under an argon atmosphere, to a 300 ml, three-neck flask, 10.00 g (22.1 mmol) of Intermediate IM-3, 0.38 g (0.03 weight equivalent, 0.7 mmol) of Pd(dba)₂, 4.26 g (2.0 weight equivalent, 44.3 mmol) of NaOtBu, 110 ml of toluene, 7.53 g (1.1 weight equivalent, 24.4 mmol) of 4-bromo-1,1′:2′,1″-terphenyl and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound B9 (11.59 g, yield 77%) as a solid.

From the measurement results of FAB-MS, mass number of m/z=679 was observed as a molecular ion peak and Compound B9 was identified.

1-7. Synthesis of Compound B14

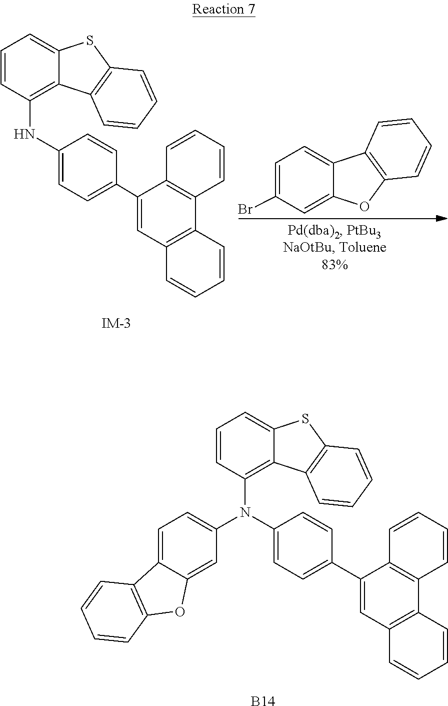

Synthesis of Compound B14

Under an argon atmosphere, to a 300 ml, three-neck flask, 10.00 g (22.1 mmol) of Intermediate IM-3, 0.38 g (0.03 weight equivalent, 0.7 mmol) of Pd(dba)₂, 4.26 g (2.0 weight equivalent, 44.3 mmol) of NaOtBu, 110 ml of toluene, 6.02 g (1.1 weight equivalent, 24.4 mmol) of 3-bromodibenzofuran and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound B14 (11.35 g, yield 83%) as a solid.

From the measurement results of FAB-MS, mass number of m/z=617 was observed as a molecular ion peak and Compound B14 was identified.

1-8. Synthesis of Compound B23

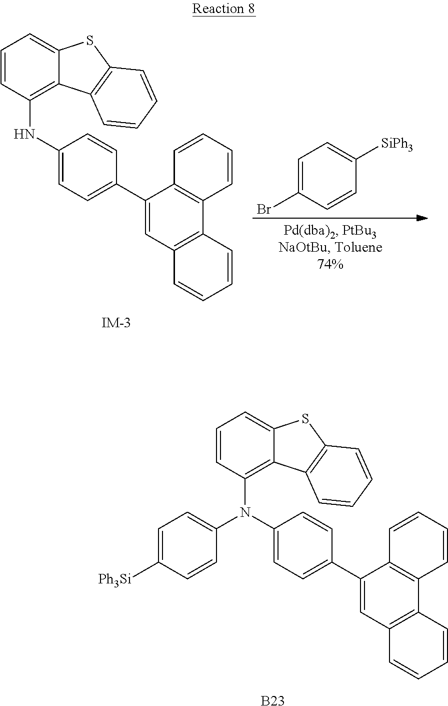

Synthesis of Compound B23

Under an argon atmosphere, to a 300 ml, three-neck flask, 10.00 g (22.1 mmol) of Intermediate IM-3, 0.38 g (0.03 weight equivalent, 0.7 mmol) of Pd(dba)₂, 4.26 g (2.0 weight equivalent, 44.3 mmol) of NaOtBu, 110 ml of toluene, 10.12 g (1.1 weight equivalent, 24.4 mmol) of (4-bromophenyl)triphenylsilane and 0.45 g (0.1 weight equivalent, 2.2 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound B23 (12.88 g, yield 74%) as a solid.

From the measurement results of FAB-MS, mass number of m/z=786 was observed as a molecular ion peak and Compound B23 was identified.

1-9. Synthesis of Compound C1

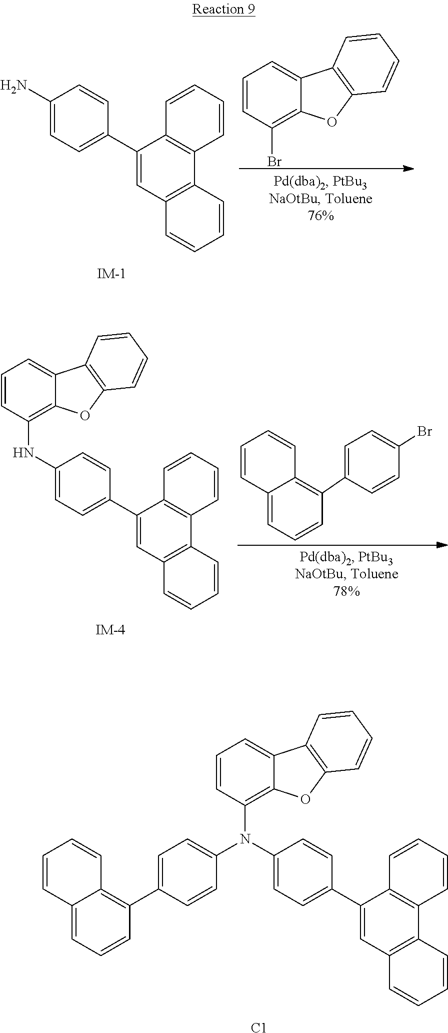

Synthesis of Intermediate IM-4

Under an argon atmosphere, to a 300 ml, three-neck flask, 10.00 g (37.1 mmol) of Intermediate IM-1, 0.64 g (0.03 weight equivalent, 1.1 mmol) of Pd(dba)₂, 3.57 g (1.0 weight equivalent, 37.1 mmol) of NaOtBu, 185 ml of toluene, 10.09 g (1.1 weight equivalent, 40.8 mmol) of 4-bromodibenzofuran and 0.75 g (0.1 weight equivalent, 3.7 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-4 (12.29 g, yield 76%).

From the measurement results of FAB-MS, mass number of m/z=435 was observed as a molecular ion peak and Intermediate IM-4 was identified.

Synthesis of Compound C1

Under an argon atmosphere, to a 300 ml, three-neck flask, 10.00 g (23.0 mmol) of Intermediate IM-4, 0.40 g (0.03 weight equivalent, 0.7 mmol) of Pd(dba)₂, 4.41 g (2.0 weight equivalent, 45.9 mmol) of NaOtBu, 115 ml of toluene, 7.15 g (1.1 weight equivalent, 25.3 mmol) of 1-(4-bromophenyl)naphthalene and 0.46 g (0.1 weight equivalent, 2.3 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound C1 (11.42 g, yield 78%) as a solid.

From the measurement results of FAB-MS, mass number of m/z=637 was observed as a molecular ion peak and Compound C1 was identified.

1-10. Synthesis of Compound C12

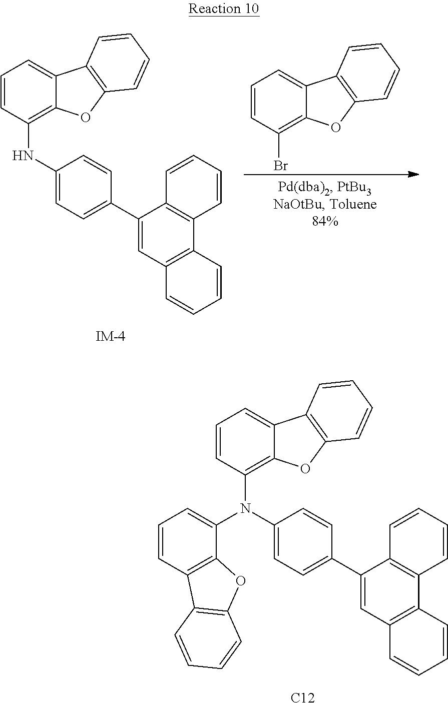

Synthesis of Compound C12

Under an argon atmosphere, to a 300 ml, three-neck flask, 10.00 g (23.0 mmol) of Intermediate IM-4, 0.40 g (0.03 weight equivalent, 0.7 mmol) of Pd(dba)₂, 4.41 g (2.0 weight equivalent, 45.9 mmol) of NaOtBu, 115 ml of toluene, 6.24 g (1.1 weight equivalent, 25.3 mmol) of 4-bromodibenzofuran and 0.46 g (0.1 weight equivalent, 2.3 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound C12 (11.60 g, yield 84%) as a solid.

From the measurement results of FAB-MS, mass number of m/z=601 was observed as a molecular ion peak and Compound C12 was identified.

1-11. Synthesis of Compound C15

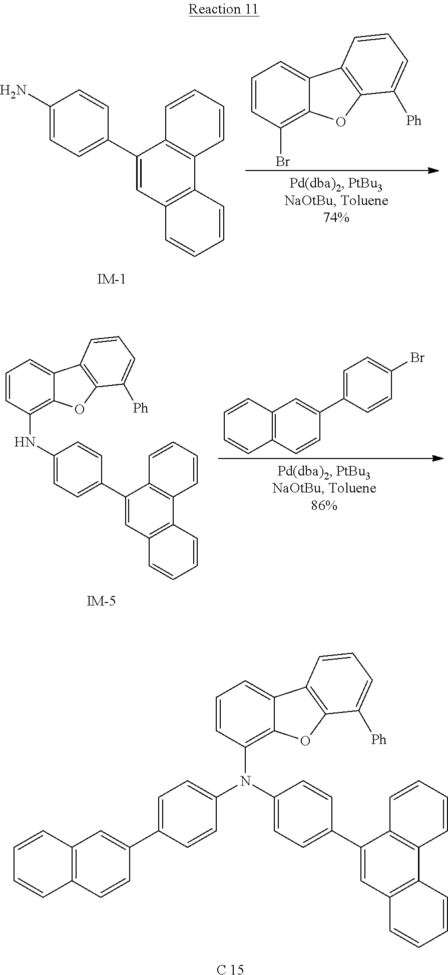

Synthesis of Intermediate IM-5

Under an argon atmosphere, to a 300 ml, three-neck flask, 10.00 g (37.1 mmol) of Intermediate IM-1, 0.64 g (0.03 weight equivalent, 1.1 mmol) of Pd(dba)₂, 3.57 g (1.0 weight equivalent, 37.1 mmol) of NaOtBu, 185 ml of toluene, 13.20 g (1.1 weight equivalent, 40.8 mmol) of 6-phenyl-4-bromodibenzofuran and 0.75 g (0.1 weight equivalent, 3.7 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-5 (14.06 g, yield 74%).

From the measurement results of FAB-MS, mass number of m/z=511 was observed as a molecular ion peak and Intermediate IM-5 was identified.

Synthesis of Compound C15

Under an argon atmosphere, to a 300 ml, three-neck flask, 10.00 g (19.5 mmol) of Intermediate IM-5, 0.34 g (0.03 weight equivalent, 0.6 mmol) of Pd(dba)₂, 3.76 g (2.0 weight equivalent, 39.1 mmol) of NaOtBu, 98 ml of toluene, 6.09 g (1.1 weight equivalent, 21.5 mmol) of 2-(4-bromophenyl)naphthalene and 0.40 g (0.1 weight equivalent, 2.0 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound C15 (12.00 g, yield 86%) as a solid.

From the measurement results of FAB-MS, mass number of m/z=713 was observed as a molecular ion peak and Compound C15 was identified.

1-12. Synthesis of Compound C21

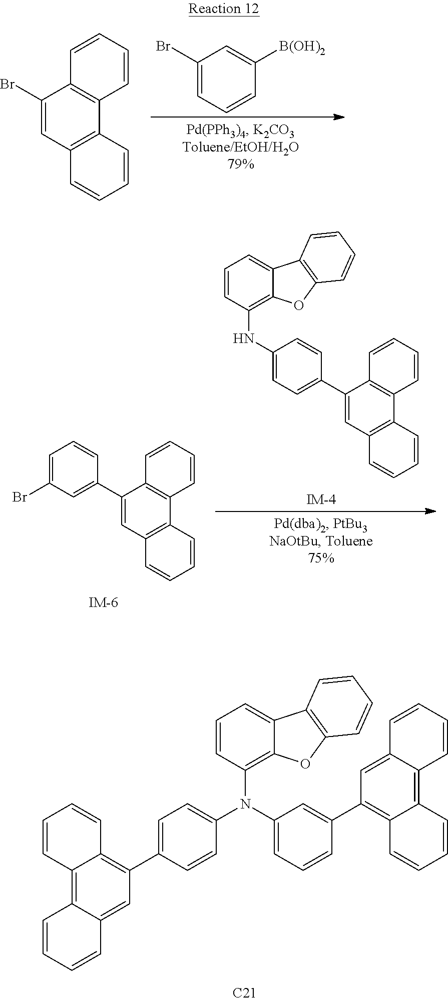

Synthesis of Intermediate IM-6

Under an argon atmosphere, to a 1,000 ml, three-neck flask, 20.00 g (77.8 mmol) of 9-bromophenanthrene, 18.75 g (1.2 weight equivalent, 93.4 mmol) of 3-bromophenylboronic acid, 32.25 g (3.0 weight equivalent, 233.3 mmol) of K₂CO₃, 4.49 g (0.05 weight equivalent, 3.9 mmol) of Pd(PPh₃)₄, and 545 ml of a mixture solution of toluene/ethanol/H₂O (a volumetric ratio of 4/2/1) were added in order, followed by heating and stirring at about 80° C. After cooling to room temperature in the air, the reaction solution was extracted with toluene. An aqueous layer was removed, and an organic layer was washed with a saturated saline solution and dried with an anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-6 (20.48 g, yield 79%).

From the measurement results of FAB-MS, mass number of m/z=333 was observed as a molecular ion peak and Intermediate IM-6 was identified.

Synthesis of Compound C21

Under an argon atmosphere, to a 300 ml, three-neck flask, 10.00 g (19.5 mmol) of Intermediate IM-5, 0.34 g (0.03 weight equivalent, 0.6 mmol) of Pd(dba)₂, 3.76 g (2.0 weight equivalent, 39.1 mmol) of NaOtBu, 98 ml of toluene, 7.16 g (1.1 weight equivalent, 21.5 mmol) of IM-6 and 0.40 g (0.1 weight equivalent, 2.0 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution, and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound C21 (10.08 g, yield 75%) as a solid.

From the measurement results of FAB-MS, mass number of m/z=687 was observed as a molecular ion peak and Compound C21 was identified.

1-13. Synthesis of Compound D5

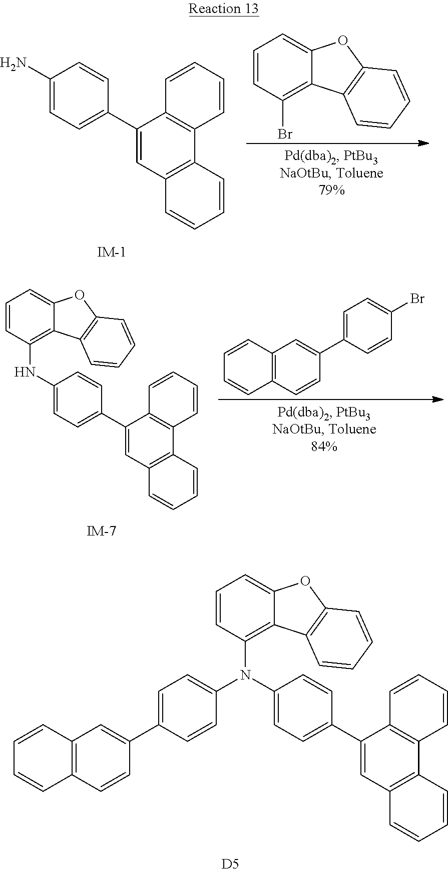

Synthesis of Intermediate IM-7

Under an argon atmosphere, to a 300 ml, three-neck flask, 10.00 g (37.1 mmol) of Intermediate IM-1, 0.64 g (0.03 weight equivalent, 1.1 mmol) of Pd(dba)₂, 3.57 g (1.0 weight equivalent, 37.1 mmol) of NaOtBu, 185 ml of toluene, 10.09 g (1.1 weight equivalent, 40.8 mmol) of 1-bromodibenzofuran and 0.75 g (0.1 weight equivalent, 3.7 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-7 (12.77 g, yield 79%).

From the measurement results of FAB-MS, mass number of m/z=435 was observed as a molecular ion peak and Intermediate IM-7 was identified.

Synthesis of Compound D5

Under an argon atmosphere, to a 300 ml, three-neck flask, 10.00 g (23.0 mmol) of Intermediate IM-7, 0.40 g (0.03 weight equivalent, 0.7 mmol) of Pd(dba)₂, 4.41 g (2.0 weight equivalent, 45.9 mmol) of NaOtBu, 115 ml of toluene, 7.15 g (1.1 weight equivalent, 25.3 mmol) of 2-(4-bromophenyl)naphthalene and 0.46 g (0.1 weight equivalent, 2.3 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound D5 (12.30 g, yield 84%) as a solid.

From the measurement results of FAB-MS, mass number of m/z=637 was observed as a molecular ion peak and Compound D5 was identified.

1-14. Synthesis of Compound D6

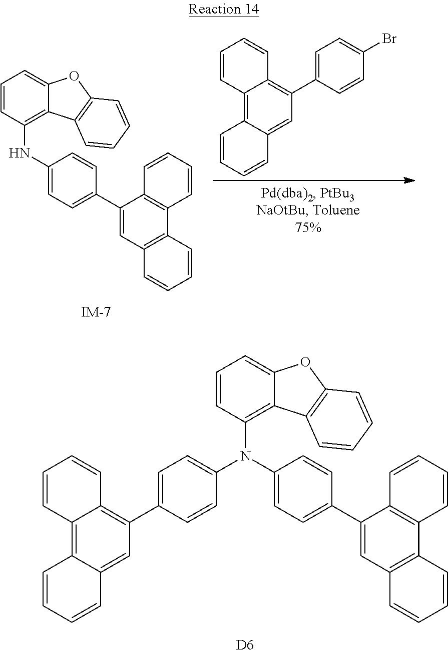

Synthesis of Compound D6

Under an argon atmosphere, to a 300 ml, three-neck flask, 10.00 g (23.0 mmol) of Intermediate IM-7, 0.40 g (0.03 weight equivalent, 0.7 mmol) of Pd(dba)₂, 4.41 g (2.0 weight equivalent, 45.9 mmol) of NaOtBu, 115 ml of toluene, 8.42 g (1.1 weight equivalent, 25.3 mmol) of 9-(4-bromophenyl)phenanthrene and 0.46 g (0.1 weight equivalent, 2.3 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution, and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound D6 (11.84 g, yield 75%) as a solid.

From the measurement results of FAB-MS, mass number of m/z=687 was observed as a molecular ion peak and Compound D6 was identified.

1-15. Synthesis of Compound D13

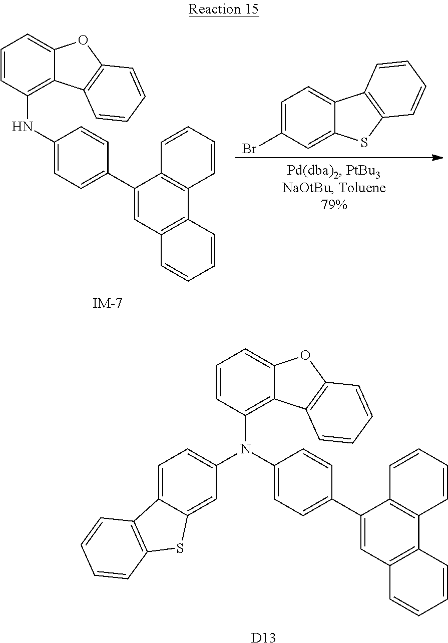

Synthesis of Compound D13

Under an argon atmosphere, to a 300 ml, three-neck flask, 10.00 g (23.0 mmol) of Intermediate IM-7, 0.40 g (0.03 weight equivalent, 0.7 mmol) of Pd(dba)₂, 4.41 g (2.0 weight equivalent, 45.9 mmol) of NaOtBu, 115 ml of toluene, 6.65 g (1.1 weight equivalent, 25.3 mmol) of 3-bromodibenzothiophene and 0.46 g (0.1 weight equivalent, 2.3 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound D13 (11.21 g, yield 79%) as a solid.

From the measurement results of FAB-MS, mass number of m/z=617 was observed as a molecular ion peak and Compound D13 was identified.

1-16. Synthesis of Compound D24

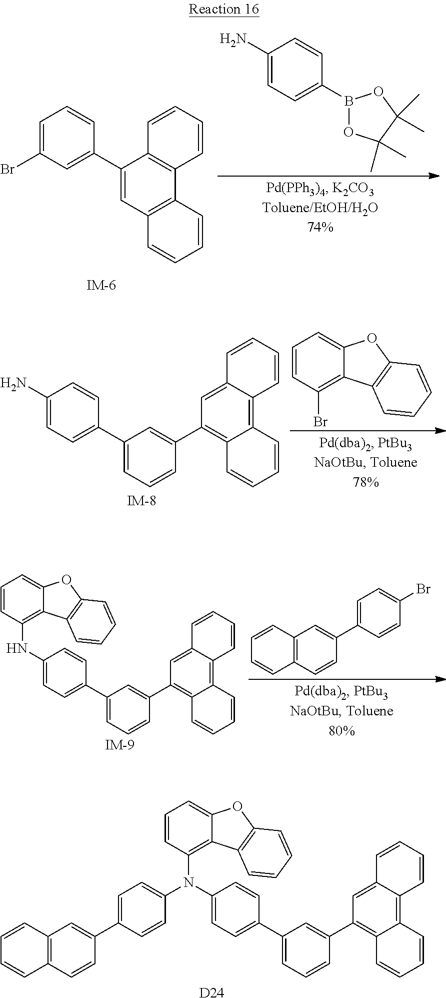

Synthesis of Intermediate IM-8

Under an argon atmosphere, to a 1,000 ml, three-neck flask, 20.00 g (60.0 mmol) of IM-6, 15.78 g (1.2 weight equivalent, 72.0 mmol) of 4-(4,4,5,5-tetramethyl-1,3,2-dioxaborolan-2-yl)aniline, 24.89 g (3.0 weight equivalent, 180.0 mmol) of K₂CO₃, 3.47 g (0.05 weight equivalent, 3.0 mmol) of Pd(PPh₃)₄, and 420 ml of a mixture solution of toluene/ethanol/H₂O (a volumetric ratio of 4/2/1) were added in order, followed by heating and stirring at about 80° C. After cooling to room temperature in the air, the reaction solution was extracted with toluene. An aqueous layer was removed, and an organic layer was washed with a saturated saline solution and dried with an anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-6 (15.34 g, yield 74%).

From the measurement results of FAB-MS, mass number of m/z=345 was observed as a molecular ion peak and Intermediate IM-8 was identified.

Synthesis of Intermediate IM-9

Under an argon atmosphere, to a 300 ml, three-neck flask, 10.00 g (28.9 mmol) of Intermediate IM-8, 0.50 g (0.03 weight equivalent, 0.9 mmol) of Pd(dba)₂, 2.78 g (1.0 weight equivalent, 28.9 mmol) of NaOtBu, 145 ml of toluene, 7.87 g (1.1 weight equivalent, 11.55 mmol) of 1-bromodibenzofuran and 0.59 g (0.1 weight equivalent, 2.9 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Intermediate IM-9 (11.55 g, yield 78%).

From the measurement results of FAB-MS, mass number of m/z=511 was observed as a molecular ion peak and Intermediate IM-9 was identified.

Synthesis of Compound D24

Under an argon atmosphere, to a 300 ml, three-neck flask, 10.00 g (19.5 mmol) of Intermediate IM-9, 0.34 g (0.03 weight equivalent, 0.6 mmol) of Pd(dba)₂, 3.76 g (2.0 weight equivalent, 39.1 mmol) of NaOtBu, 98 ml of toluene, 6.09 g (1.1 weight equivalent, 21.5 mmol) of 2-(4-bromophenyl)naphthalene and 0.40 g (0.1 weight equivalent, 2.0 mmol) of tBu₃P were added in order, followed by heating, refluxing and stirring. After cooling to room temperature in the air, water was added to the reaction solvent and an organic layer was separately taken. Toluene was added to an aqueous layer and an organic layer was further extracted. Organic layers were collected, washed with a saline solution, and dried with anhydrous magnesium sulfate. The anhydrous magnesium sulfate was strained and an organic layer was concentrated. The crude product thus obtained was separated by silica gel column chromatography (using a mixture solvent of hexane and toluene as a developing layer) to obtain Compound D24 (9.66 g, yield 80%) as a solid.

From the measurement results of FAB-MS, mass number of m/z=713 was observed as a molecular ion peak and Compound D24 was identified.

### 2. Manufacture and Evaluation of Luminescence Device Including Amine Compound

### 2-1. Examples of Luminescence Devices Including Amine Compounds

Luminescence devices of Examples 1 to 16, and Comparative Examples 1 to 10 were manufactured using Example Compounds A1, A7, A12, A18, B5, B9, B14, B23, C1, C12, C15, C21, D5, D6, D13, and D24, and Comparative Compounds R1 to R10 as materials for a hole transport layer.

### Example Compounds

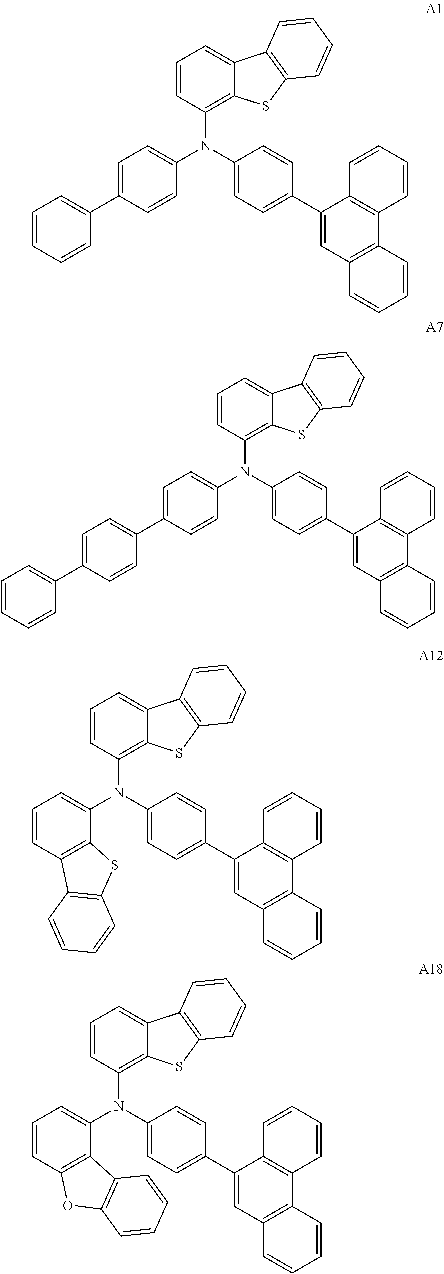 [SMILES: c1ccc(-c2ccc(N(c3ccc(-c4cc5ccccc5c5ccccc45)cc3)c3cccc4c3sc3ccccc34)cc2)cc1 ; c1ccc(-c2ccc(-c3ccc(N(c4ccc(-c5cc6ccccc6c6ccccc56)cc4)c4cccc5c4sc4ccccc45)cc3)cc2)cc1 ; c1ccc2c(c1)cc(-c1ccc(N(c3cccc4c3sc3ccccc34)c3cccc4c3sc3ccccc34)cc1)c1ccccc12 ; c1ccc2c(c1)cc(-c1ccc(N(c3cccc4c3sc3ccccc34)c3cccc4oc5ccccc5c34)cc1)c1ccccc12]

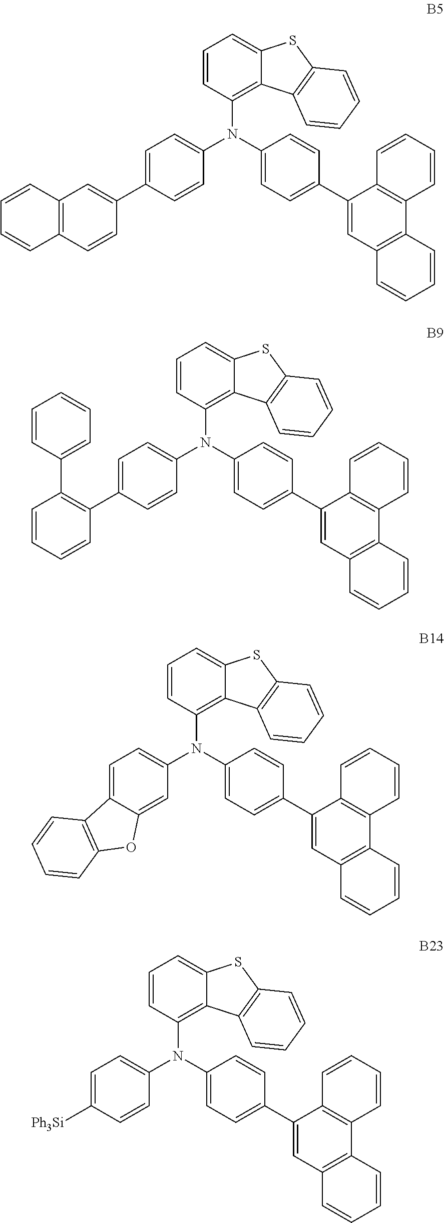

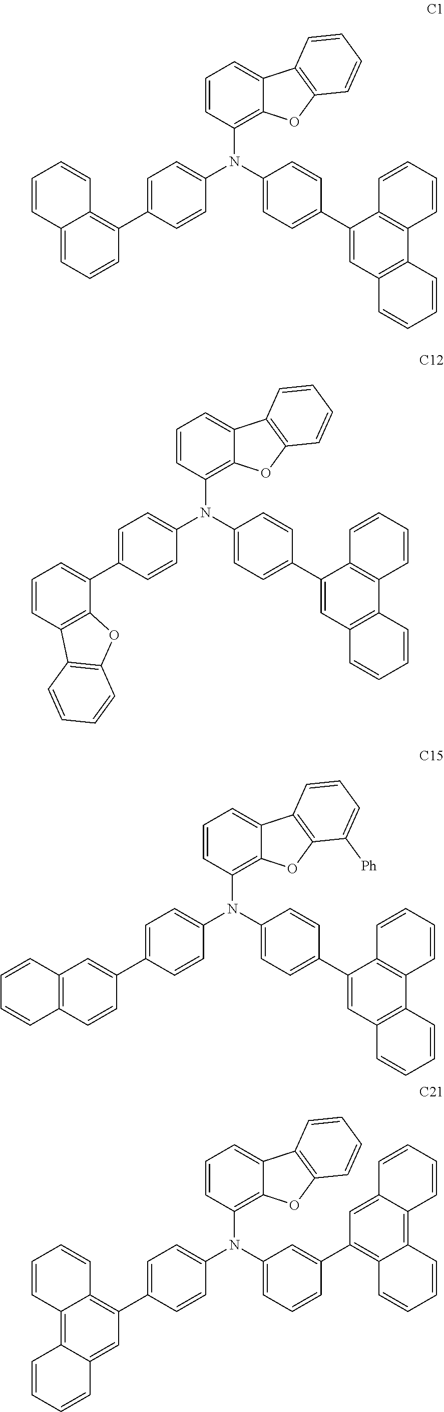 [SMILES: c1ccc2c(-c3ccc(N(c4ccc(-c5cc6ccccc6c6ccccc56)cc4)c4cccc5c4oc4ccccc45)cc3)cccc2c1 ; c1ccc2c(c1)cc(-c1ccc(N(c3ccc(-c4cccc5c4oc4ccccc45)cc3)c3cccc4c3oc3ccccc34)cc1)c1ccccc12 ; c1ccc(-c2cccc3c2oc2c(N(c4ccc(-c5ccc6ccccc6c5)cc4)c4ccc(-c5cc6ccccc6c6ccccc56)cc4)cccc23)cc1 ; c1cc(-c2cc3ccccc3c3ccccc23)cc(N(c2ccc(-c3cc4ccccc4c4ccccc34)cc2)c2cccc3c2oc2ccccc23)c1]

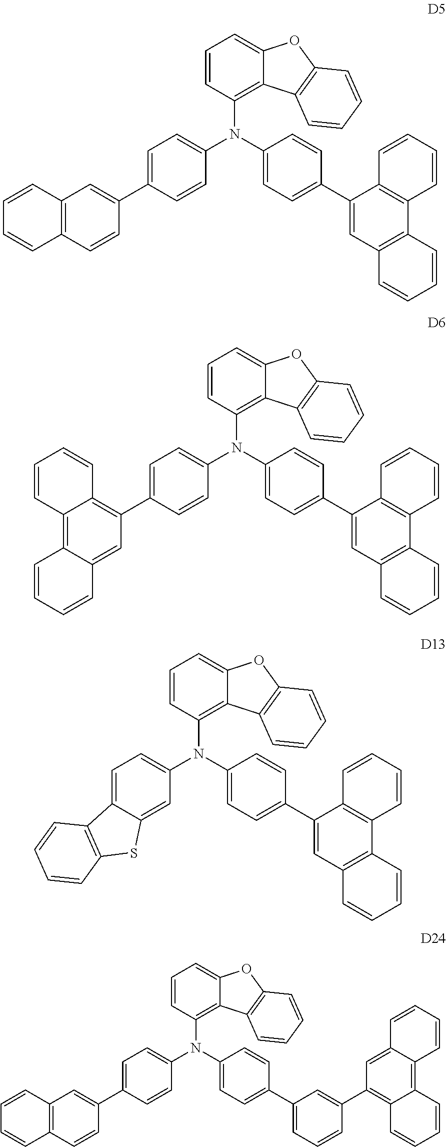 [SMILES: c1ccc2cc(-c3ccc(N(c4ccc(-c5cc6ccccc6c6ccccc56)cc4)c4cccc5oc6ccccc6c45)cc3)ccc2c1 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc2c(c1)cc(-c1ccc(N(c3ccc4c(c3)sc3ccccc34)c3cccc4oc5ccccc5c34)cc1)c1ccccc12 ; c1cc(-c2ccc(N(c3ccc(-c4ccc5ccccc5c4)cc3)c3cccc4oc5ccccc5c34)cc2)cc(-c2cc3ccccc3c3ccccc23)c1]

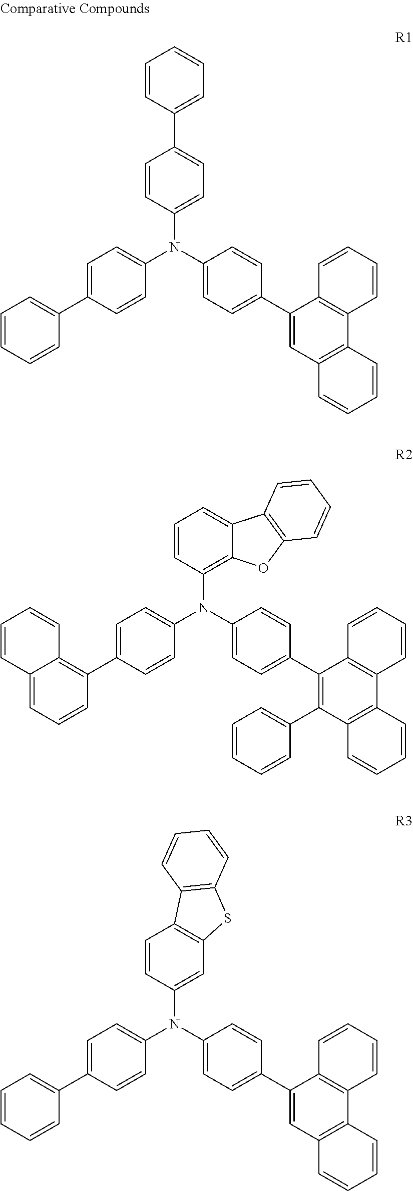 [SMILES: c1ccc(-c2ccc(N(c3ccc(-c4ccccc4)cc3)c3ccc(-c4cc5ccccc5c5ccccc45)cc3)cc2)cc1 ; c1ccc(-c2c(-c3ccc(N(c4ccc(-c5cccc6ccccc56)cc4)c4cccc5c4oc4ccccc45)cc3)c3ccccc3c3ccccc23)cc1 ; c1ccc(-c2ccc(N(c3ccc(-c4cc5ccccc5c5ccccc45)cc3)c3ccc4c(c3)sc3ccccc34)cc2)cc1]

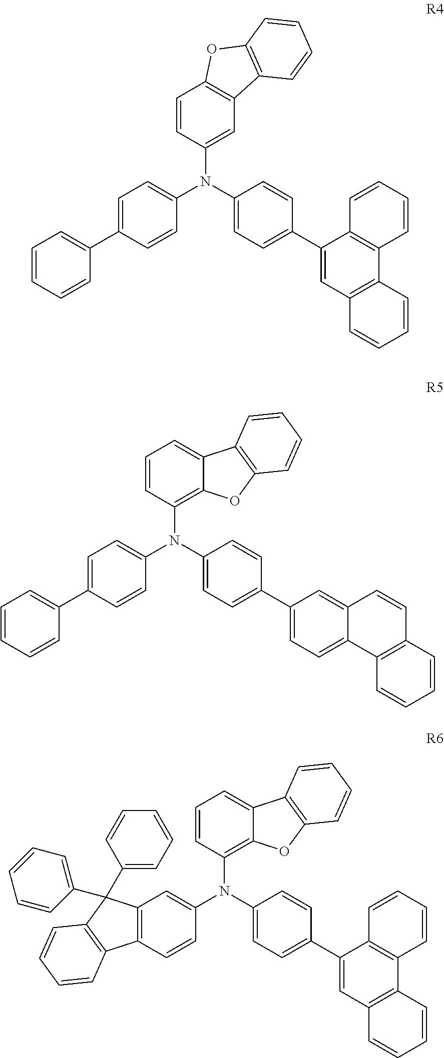 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4ccc5c(ccc6ccccc65)c4)cc3)c3cccc4c3oc3ccccc34)cc2)cc1 ; c1ccc(C2(c3ccccc3)c3ccccc3-c3ccc(N(c4ccc(-c5cc6ccccc6c6ccccc56)cc4)c4cccc5c4oc4ccccc45)cc32)cc1]

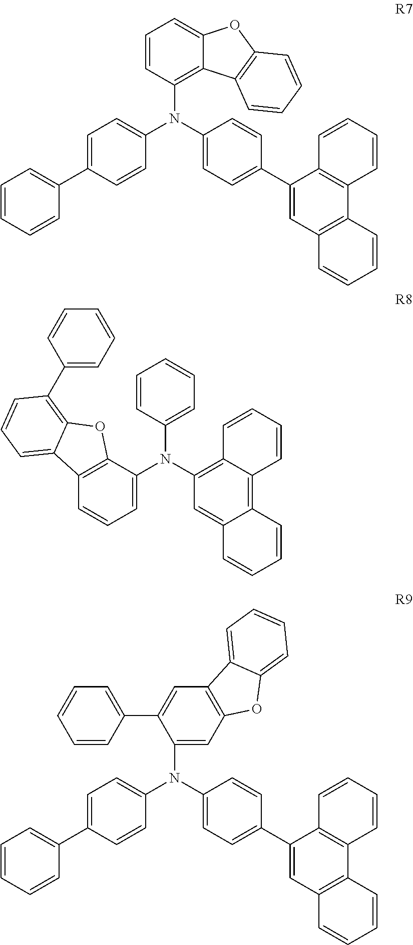

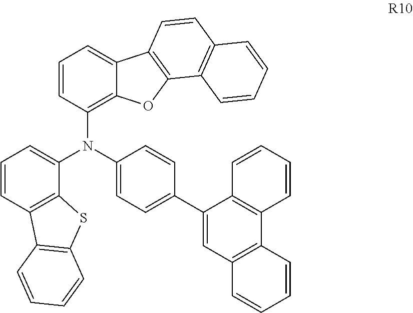 [SMILES: c1ccc2c(c1)cc(-c1ccc(N(c3cccc4c3oc3c5ccccc5ccc43)c3cccc4c3sc3ccccc34)cc1)c1ccccc12]

Manufacture of Luminescence Device

Each of the luminescence devices of Examples 1 to 16 and Comparative Examples 1 to 10 was manufactured as follows. A first electrode EL1 with a thickness of about 150 nm was formed using an ITO. A hole injection layer HIL with a thickness of about 60 nm was formed using (4,4′,4″-tris{N,-1-naphthyl)-N-phenylamino}-triphenylamine (1-TNATA), and a hole transport layer HTL with a thickness of about 30 nm was formed using each of the Example Compounds and Comparative Compounds. An emission layer EML with a thickness of about 25 nm was formed using 9,10-bis(2-naphthyl)anthrace (ADN) doped with 3% (mole percent) TBP. An electron transport layer ETL with a thickness of about 25 nm was formed using Alq3, and an electron injection layer EIL with a thickness of about 1 nm was formed using LiF. A second electrode EL2 with a thickness of about 100 nm was formed using Al. All layers were formed by a vacuum deposition method. Commercially available materials of 1-TNATA, TBP, ADN, and Alq3 were used after conducting sublimation and purification.

Evaluation of Properties of Luminescence Device

In order to evaluate the properties of the luminescence devices 10 according to the Examples and Comparative Examples, a driving voltage, current efficiency, and luminance half life (LT50) were measured. The current efficiency is a value on a current density of about 10 mA/cm². The current density of the luminance half life was measured by continuously driving at about 1.0 mA/cm². In order to evaluate the light-emitting properties of the luminescence devices 10 thus manufactured, current density, a driving voltage, and emission efficiency were measured using a source meter of 2400 Series of Keithley Instruments Co. a company affiliated with Tektronix of Beaverton, Oreg., a luminance colorimeter, sold under the trade designation CS-200, which is a product of Konica Minolta Co. of Tokyo, Japan, and PC Program sold under the trade designation LabVIEW8.2 for measurement, which is a product of National Instruments Co., Minato-ku, Japan.

TABLE 1

| Device | Hole | | | |
|---|---|---|---|---|
| Manu- | Transport | Driving | Current | Luminance |
| facturing | Layer | Voltage | Efficiency | Half |
| Example | Material | (V) | (cd/A) | Life (Hour) |
| Example 1 | Example Compound A1 | 5.4 | 7.5 | 1950 |
| Example 2 | Example Compound A7 | 5.5 | 7.5 | 2000 |
| Example 3 | Example Compound A12 | 5.4 | 7.8 | 1900 |
| Example 4 | Example Compound A18 | 5.4 | 7.9 | 1800 |
| Example 5 | Example Compound B5 | 5.6 | 7.6 | 1900 |
| Example 6 | Example Compound B9 | 5.6 | 7.6 | 1900 |
| Example 7 | Example Compound B14 | 5.6 | 7.6 | 1850 |
| Example 8 | Example Compound B23 | 5.6 | 7.7 | 1800 |
| Example 9 | Example Compound C1 | 5.6 | 7.6 | 1950 |
| Example 10 | Example Compound C12 | 5.4 | 7.8 | 2050 |
| Example 11 | Example Compound C15 | 5.5 | 7.6 | 2000 |
| Example 12 | Example Compound C21 | 5.6 | 7.7 | 1900 |
| Example 13 | Example Compound D5 | 5.7 | 7.7 | 1900 |
| Example 14 | Example Compound D6 | 5.6 | 7.8 | 1850 |
| Example 15 | Example Compound D13 | 5.6 | 7.6 | 1900 |
| Example 16 | Example Compound D24 | 5.5 | 7.8 | 1850 |
| Comparative | Comparative Compound | 6.3 | 6.0 | 1700 |
| Example 1 | R1 | | | |
| Comparative | Comparative Compound | 6.0 | 6.5 | 1600 |
| Example 2 | R2 | | | |
| Comparative | Comparative Compound | 6.3 | 6.3 | 1550 |
| Example 3 | R3 | | | |
| Comparative | Comparative Compound | 6.3 | 6.4 | 1750 |
| Example 4 | R4 | | | |
| Comparative | Comparative Compound | 6.4 | 6.5 | 1650 |
| Example 5 | R5 | | | |
| Comparative | Comparative Compound | 6.3 | 6.7 | 1700 |
| Example 6 | R6 | | | |
| Comparative | Comparative Compound | 5.9 | 7.2 | 1750 |
| Example 7 | R7 | | | |
| Comparative | Comparative Compound | 6.4 | 7.3 | 1600 |
| Example 8 | R8 | | | |
| Comparative | Comparative Compound | 6.5 | 7.4 | 1600 |
| Example 9 | R9 | | | |
| Comparative | Comparative Compound | 6.5 | 7.3 | 1650 |
| Example 10 | R10 | | | |

Referring to the results of Table 1, it can be seen that if the amine compounds according to exemplary embodiments are applied to a luminescence device as a material for a hole transport layer, a low driving voltage, high efficiency, and long life can be achieved. Particularly, it was confirmed that Example 1 to Example 16 achieved a lower driving voltage, higher efficiency, and longer life when compared with Comparative Example 1 to Comparative Example 10. When the amine compound of some exemplary embodiments includes a phenanthrene skeleton and a dibenzohetero (particularly, dibenzofuran or dibenzothiophene) skeleton, and the low driving voltage, long life, and high efficiency could be achieved.

Although not wanting to be bound by theory, in the amine compound of some exemplary embodiments, it is considered that the energy level of the highest occupied molecular orbital (HOMO) becomes deepen by combining carbon at position 1 (or position 8, hereinafter, will be described as position 1), or carbon at position 4 (or position 5, hereinafter, will be described as position 4) of a dibenzoheterocyclic group with a nitrogen atom. Accordingly, the hole transport properties of a hole transport layer HTL is improved, and thus, the recombination probability of holes and electrons in an emission layer EML is increased and the emission efficiency is improved. Further, the volume of a molecule is increased, and crystallinity is restrained by the combination of the carbon at position 1 (or position 8), or carbon at position 4 (or position 5) of the dibenzoheterocyclic group in the amine compound of some exemplary embodiments, and the layer forming properties of the hole transport layer is improved.

When comparing Examples 1 to 4, and 9 to 12, where position 1 of the dibenzoheterocyclic group is combined with the nitrogen atom with Examples 5 to 8, and 13 to 16, where position 4 of the dibenzoheterocyclic group is combined with the nitrogen atom, Examples 1 to 4, and 9 to 12 showed longer life by a significant degree. Although not wanting to be bound by theory, position 1 of the dibenzoheterocyclic group is combined with the nitrogen atom a deeper HOMO level than a case where position 4 of the dibenzoheterocyclic group is combined with the nitrogen atom. In case where position 4 of the dibenzoheterocyclic group is combined with the nitrogen atom, the HOMO level becomes somewhat shallower, but the volume around the nitrogen atom is increased, and accordingly, the layer forming properties of a hole transport layer HTL is improved, and both emission efficiency and device life are excellent.

Although not wanting to be bound by theory in discussing the comparative examples, because the amine compound of Comparative Example 1 does not include a dibenzohetero skeleton, hole transport properties may be deteriorated, and a low driving voltage, high efficiency, and long life could not be achieved. In Comparative Example 2, because a phenyl group is included in the carbon at position 10 of a phenanthrene skeleton, the volume around a nitrogen atom is excessively increased, the bond between atoms may become unstable and may be easily deteriorated, and accordingly, the low driving voltage, high efficiency, and long life could not be achieved.

In Comparative Examples 3 and 4, by the combination of the carbon at position 2 or the carbon at position 3 of a dibenzoheterocyclic group with a nitrogen atom, the HOMO energy level becomes shallow, the volume around the nitrogen atom is reduced, the hole transport properties and layer forming properties are degraded, and the low driving voltage, high efficiency, and long life could not be achieved.

In Comparative Example 5, the carbon at position 2 of a phenanthrene skeleton is combined with a nitrogen atom. Since carbon at position 9 or position 10, which has high reactivity, is not protected, a compound is easily deteriorated, and the low driving voltage, high efficiency, and long life could not be achieved.

In Comparative Example 6, a fluorenyl group having more unstable SP³ hybrid carbon than SP² hybrid carbon is included, a compound is easily deteriorated, and the low driving voltage, high efficiency, and long life could not be achieved.

In Comparative Example 7, the carbon number of a group corresponding to Ar₁ of Formula 1 is 15 or less. Accordingly, due to the small molecular weight of the compound, a material may be decomposed during the continuous driving process of a device. Accordingly, the low driving voltage, high efficiency, and long life could not be achieved.

In Comparative Example 8, a phenanthrene group is directly combined with a nitrogen atom without a linker, and the steric structure is different from the compound of an exemplary embodiment, and thus, the low driving voltage, high efficiency, and long life could not be achieved.

In Comparative Example 9, a phenyl group is substituted in a dibenzohetero group, and carbon at position 2 is combined with a nitrogen atom. Accordingly, the HOMO energy level becomes shallower, and the steric structure is different when compared with the compound of an exemplary embodiment, and the low driving voltage, high efficiency, and long life could not be achieved.

Comparative Example 10 has a benzo structure obtained by additionally condensing a benzene ring to a dibenzohetero skeleton. Accordingly, the planarity of a molecule is increased, and stacking of molecules arises well. Accordingly, layer forming properties are degraded, deposition temperature is increased, and the low driving voltage, high efficiency, and long life could not be achieved.

The luminescence device of an exemplary embodiment includes an amine compound represented by Formula 1. Accordingly, the luminescence device may achieve a low driving voltage, high efficiency and long life. The amine compound may be applied to a luminescence device and achieve a low driving voltage, high efficiency, and long life.

Some of the advantages that may be achieved by exemplary implementations of the invention include a luminescence device and/or an amine compound applied to such a device achieving high efficiency and long life.

Although certain exemplary embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A luminescence device, comprising:
a first electrode;
a hole transport region disposed on the first electrode;
an emission layer disposed on the hole transport region;
an electron transport region disposed on the emission layer; and
a second electrode disposed on the electron transport region,
wherein the hole transport region comprises an amine compound of Formula 1: 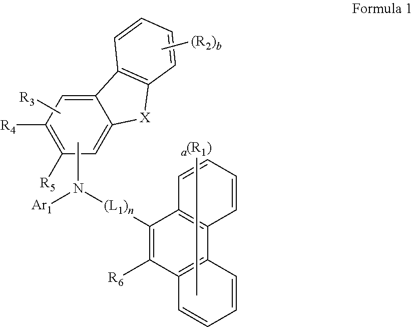
in Formula 1,
X is O or S;
R₁ to R₅ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
R₆ is a hydrogen atom or a deuterium atom;
L₁ is a substituted or unsubstituted arylene group of 6 to 40 carbon atoms for forming a ring, or a substituted or unsubstituted heteroarylene group of 2 to 40 carbon atoms for forming a ring;
Ar₁ is a substituted or unsubstituted aryl group of 6 to 40 carbon atoms for forming a ring, or a substituted or unsubstituted heteroaryl group of 2 to 12 carbon atoms for forming a ring;
if X is O, and Ar₁ is a substituted or unsubstituted aryl group with 16 to 40 carbon atoms forming a ring;
a is an integer of 0 to 8;
b is an integer of 0 to 4; and
n is an integer of 1 to 3.

2. The luminescence device of claim 1, wherein the hole transport region comprises a hole injection layer disposed on the first electrode, and a hole transport layer disposed on the hole injection layer, and
the hole transport layer comprises the amine compound.

3. The luminescence device of claim 1, wherein the emission layer comprises a polycyclic compound of Formula A: 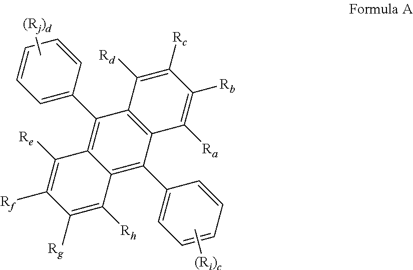
in Formula A,
Rₐ to Rⱼ are each, independently from one another, a hydrogen atom, a deuterium atom, a halogen atom, a substituted or unsubstituted silyl group, a substituted or unsubstituted alkyl group of 1 to 10 carbon atoms, a substituted or unsubstituted aryl group of 6 to 30 carbon atoms for forming a ring, or a substituted or unsubstituted heteroaryl group of 2 to 30 carbon atoms for forming a ring, or combined with an adjacent group to form a ring; and
c and d are each, independently from one another, an integer of 0 to 5.

4. The luminescence device of claim 1, wherein Ar₁ is a group of Formula 2: [symbol]
ₚ(Ar₁₁)—Ar₁₂ Formula 2
in Formula 2,
Ar₁₁ is a substituted or unsubstituted arylene group of 6 to 40 carbon atoms for forming a ring, or a substituted or unsubstituted heteroarylene group of 2 to 12 carbon atoms for forming a ring;
Ar₁₂ is a substituted or unsubstituted aryl group of 6 to 40 carbon atoms for forming a ring, or a substituted or unsubstituted heteroaryl group of 2 to 12 carbon atoms for forming a ring; and
p is 0 or 1.

5. The luminescence device of claim 1, wherein Ar₁ is a group of Formulae 1-1 to 1-10: 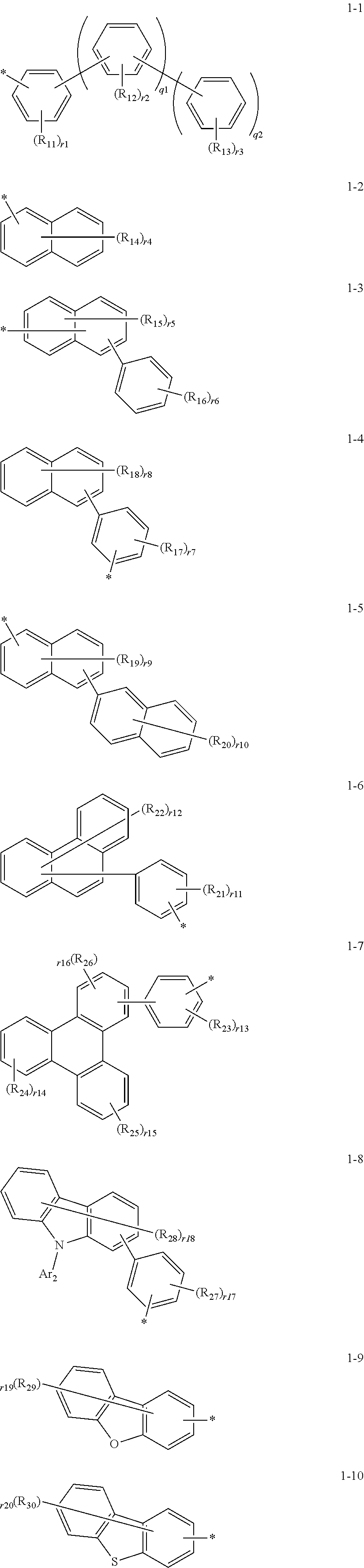
in Formulae 1-1 to 1-10,
R₁₁ to R₃₀ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
Ar₂ is a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
q1 and q2 are each, independently from one another, 0 or 1;
r1, r7, r11, and r13 to r17 are each, independently from one another, an integer of 0 to 4;
r2, r3, and r6 are each, independently from one another, an integer of 0 to 5;
r4, r8, r10, and r18 to r20 are each, independently from one another, an integer of 0 to 7;
r5 and r9 are each, independently from one another, an integer of 0 to 6; and
r12 is an integer of 0 to 9.

6. The luminescence device of claim 1, wherein Ar₁ is represented by a group of Formulae 1-11 to 1-20: 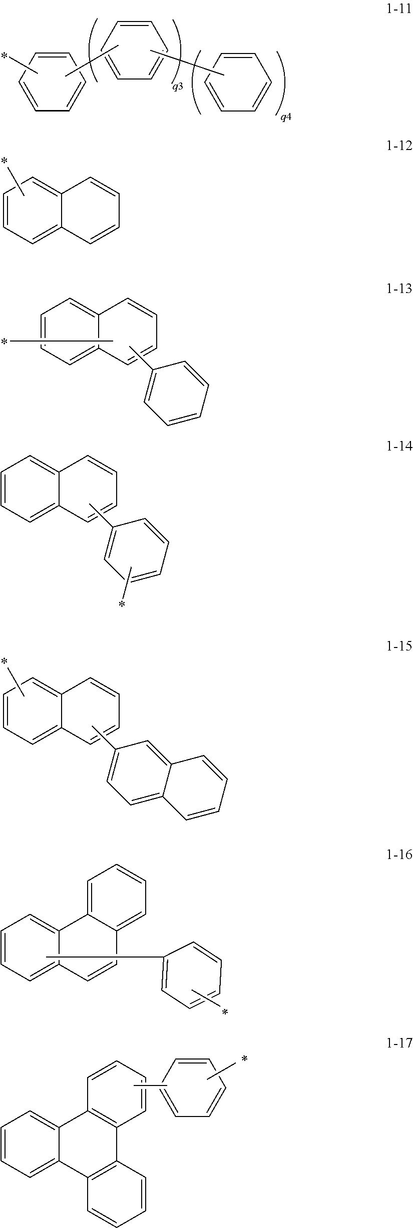 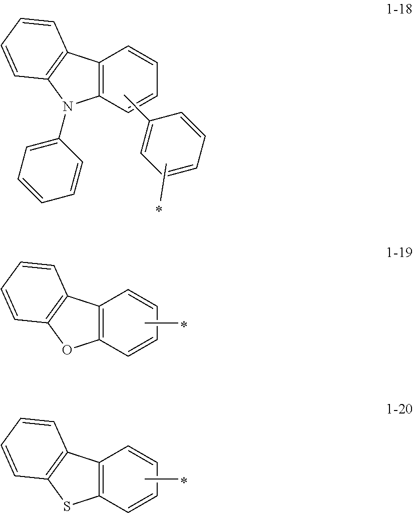
in Formula 1-11,
q3 and q4 are each, independently from one another, 0 or 1.

7. The luminescence device of claim 1, wherein L₁ is a group of Formulae 2-1 to 2-4: 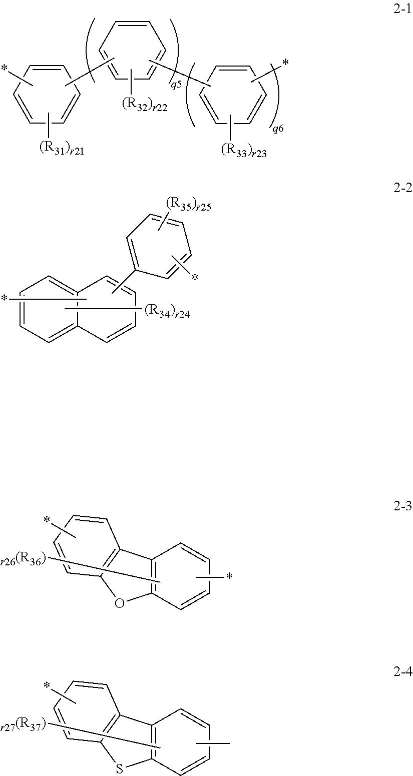
in Formulae 2-1 to 2-4,
R₃₁ to R₃₇ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
q5 and q6 are each, independently from one another, 0 or 1;
r21 to r23, and r25 are each, independently from one another; an integer of 0 to 4; and
r24, r26, and r27 are each, independently from one another, an integer of 0 to 6.

8. The luminescence device of claim 1, wherein L₁ is a group of Formulae 2-11 to 2-14: 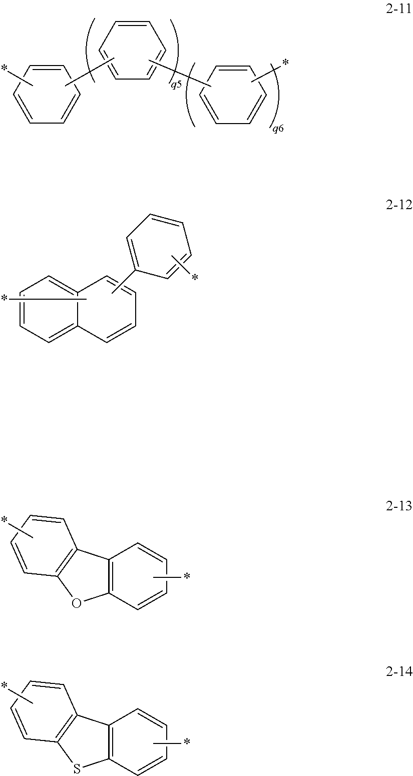
in Formulae 2-11,
q5 and q6 are each, independently from one another, 0 or 1.

9. The luminescence device of claim 1, wherein the amine compound of Formula 1 is a compound of Formula 1-1: 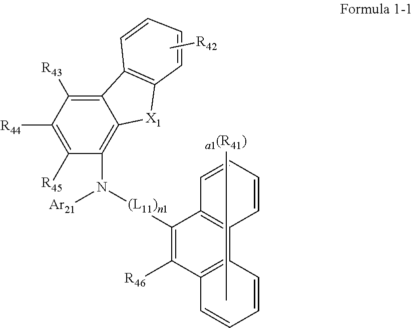
in Formula 1-1,
R₄₁ and R₄₂ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
R₄₃ to R₄₆ are each, independently from one another, a hydrogen atom or a deuterium atom;
L₁₁ is a substituted or unsubstituted arylene group of 6 to 40 carbon atoms for forming a ring, or a substituted or unsubstituted heteroarylene group of 2 to 40 carbon atoms for forming a ring;
Ar₂₁ is a substituted or unsubstituted aryl group of 6 to 40 carbon atoms for forming a ring, or a substituted or unsubstituted heteroaryl group of 2 to 12 carbon atoms for forming a ring;
if X is O, and Ar₂₁ is a substituted or unsubstituted aryl group with 16 to 40 carbon atoms forming a ring;
a1 is an integer of 0 to 8; and
n1 is an integer of 1 to 3.

10. The luminescence device of claim 1, wherein the amine compound of Formula 1 is a compound represented by the following Formula 1-2: 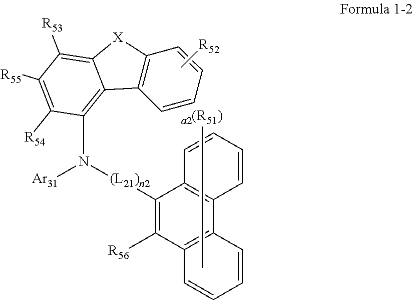
in Formula 1-2,
R₅₁ and R₅₂ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl amine group of 6 to 40 carbon atoms for forming a ring, or a substituted or unsubstituted heteroaryl group of 2 to 40 carbon atoms for forming a ring;
R₅₃ to R₅₆ are each, independently from one another, a hydrogen atom or a deuterium atom;
L₂₁ is a substituted or unsubstituted arylene group of 6 to 40 carbon atoms for forming a ring, or a substituted or unsubstituted heteroarylene group of 2 to 40 carbon atoms for forming a ring;
Ar₃₁ is a substituted or unsubstituted aryl group of 6 to 40 carbon atoms for forming a ring, or a substituted or unsubstituted heteroaryl group of 2 to 12 carbon atoms for forming a ring;
if X₂ is O, and Ar₃₁ is a substituted or unsubstituted aryl group with 16 to 40 carbon atoms forming a ring;
a2 is an integer of 0 to 8, and
n2 is an integer of 1 to 3.

11. The luminescence device of claim 1, wherein the amine compound of Formula 1 comprises at least one compound from Compound Groups A-D:

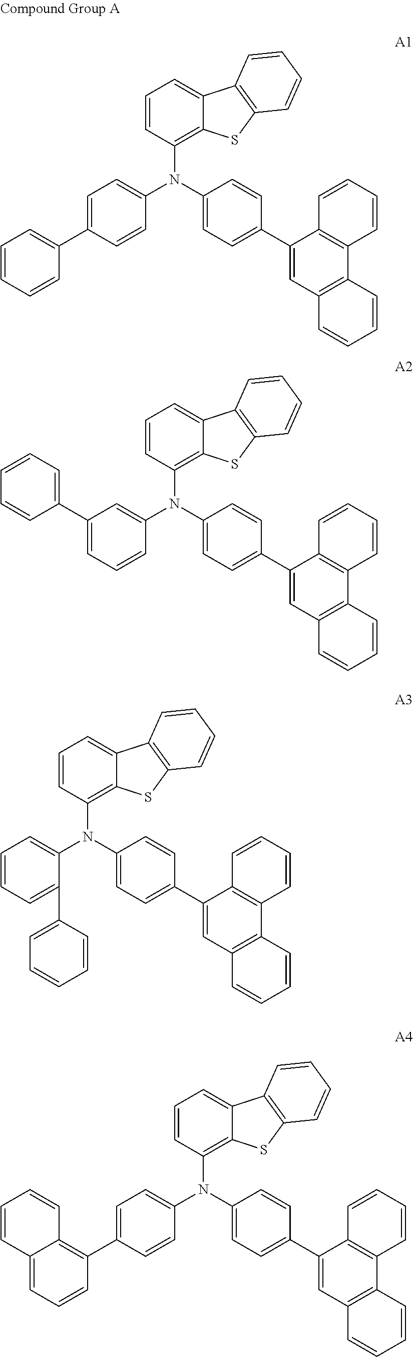 [SMILES: c1ccc(-c2ccc(N(c3ccc(-c4cc5ccccc5c5ccccc45)cc3)c3cccc4c3sc3ccccc34)cc2)cc1 ; c1ccc(-c2cccc(N(c3ccc(-c4cc5ccccc5c5ccccc45)cc3)c3cccc4c3sc3ccccc34)c2)cc1 ; c1ccc(-c2ccccc2N(c2ccc(-c3cc4ccccc4c4ccccc34)cc2)c2cccc3c2sc2ccccc23)cc1 ; c1ccc2c(-c3ccc(N(c4ccc(-c5cc6ccccc6c6ccccc56)cc4)c4cccc5c4sc4ccccc45)cc3)cccc2c1]

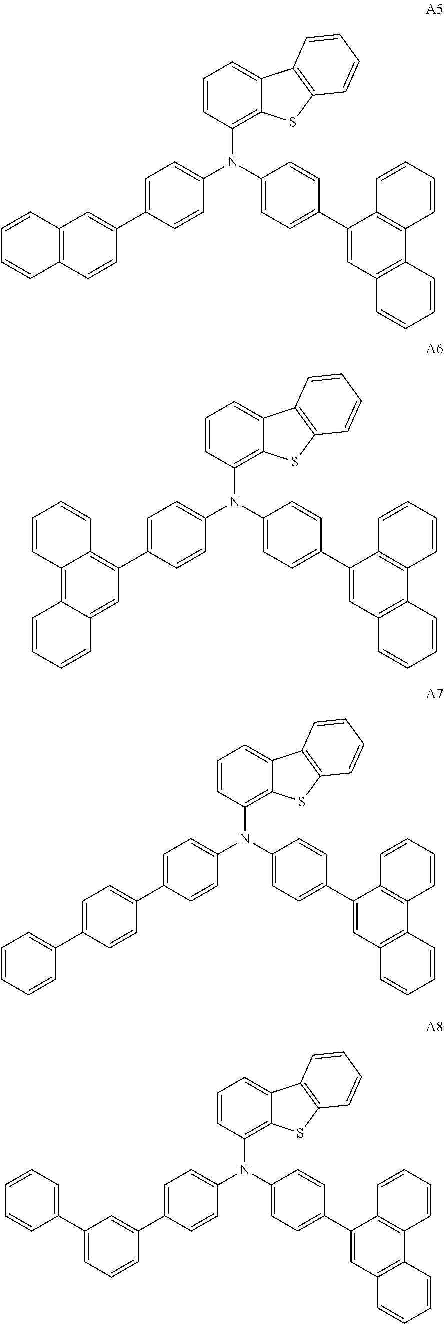 [SMILES: c1ccc2cc(-c3ccc(N(c4ccc(-c5cc6ccccc6c6ccccc56)cc4)c4cccc5c4sc4ccccc45)cc3)ccc2c1 ; c1ccc2c(c1)cc(-c1ccc(N(c3ccc(-c4cc5ccccc5c5ccccc45)cc3)c3cccc4c3sc3ccccc34)cc1)c1ccccc12 ; c1ccc(-c2ccc(-c3ccc(N(c4ccc(-c5cc6ccccc6c6ccccc56)cc4)c4cccc5c4sc4ccccc45)cc3)cc2)cc1 ; c1ccc(-c2cccc(-c3ccc(N(c4ccc(-c5cc6ccccc6c6ccccc56)cc4)c4cccc5c4sc4ccccc45)cc3)c2)cc1]

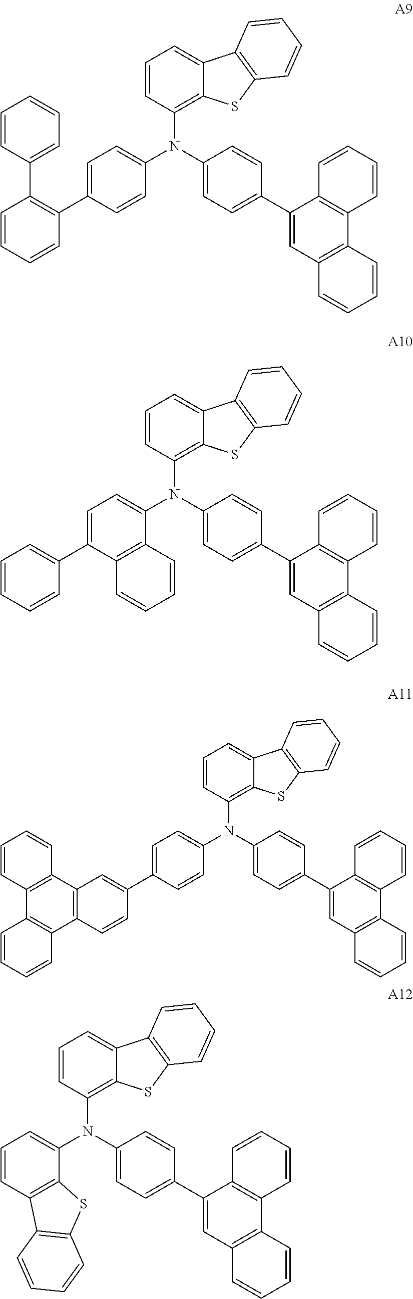 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)c3ccccc23)cc1 ; c1ccc2c(c1)cc(-c1ccc(N(c3ccc(-c4ccc5c6ccccc6c6ccccc6c5c4)cc3)c3cccc4c3sc3ccccc34)cc1)c1ccccc12 ; c1ccc2c(c1)cc(-c1ccc(N(c3cccc4c3sc3ccccc34)c3cccc4c3sc3ccccc34)cc1)c1ccccc12]

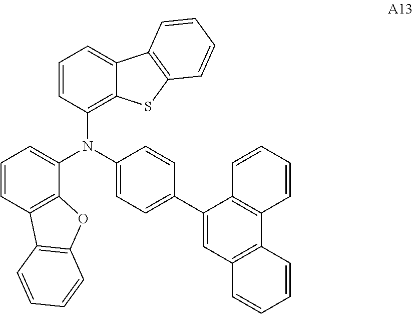 [SMILES: c1ccc2c(c1)cc(-c1ccc(N(c3cccc4c3oc3ccccc34)c3cccc4c3sc3ccccc34)cc1)c1ccccc12]

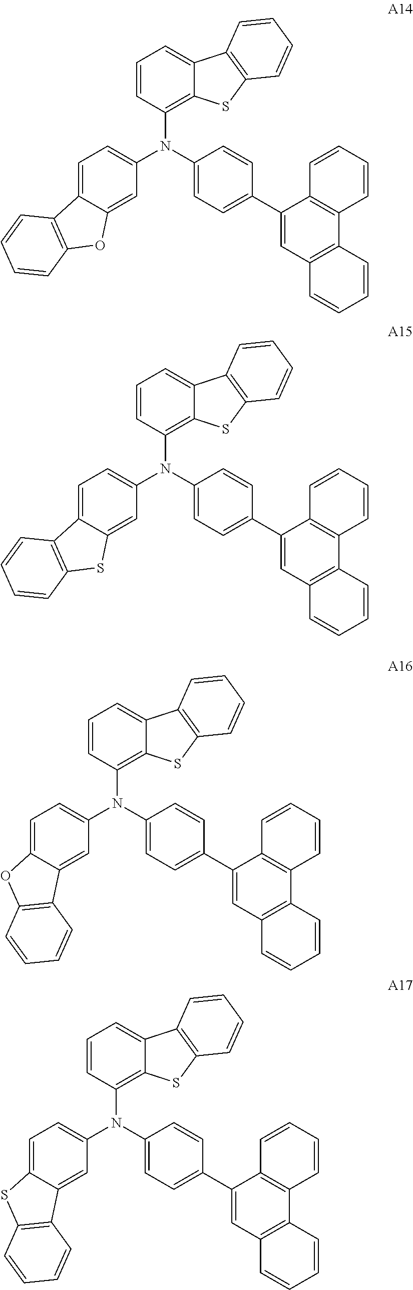 [SMILES: c1ccc2c(c1)cc(-c1ccc(N(c3ccc4c(c3)oc3ccccc34)c3cccc4c3sc3ccccc34)cc1)c1ccccc12 ; c1ccc2c(c1)cc(-c1ccc(N(c3ccc4c(c3)sc3ccccc34)c3cccc4c3sc3ccccc34)cc1)c1ccccc12 ; c1ccc2c(c1)cc(-c1ccc(N(c3ccc4oc5ccccc5c4c3)c3cccc4c3sc3ccccc34)cc1)c1ccccc12 ; c1ccc2c(c1)cc(-c1ccc(N(c3ccc4sc5ccccc5c4c3)c3cccc4c3sc3ccccc34)cc1)c1ccccc12]

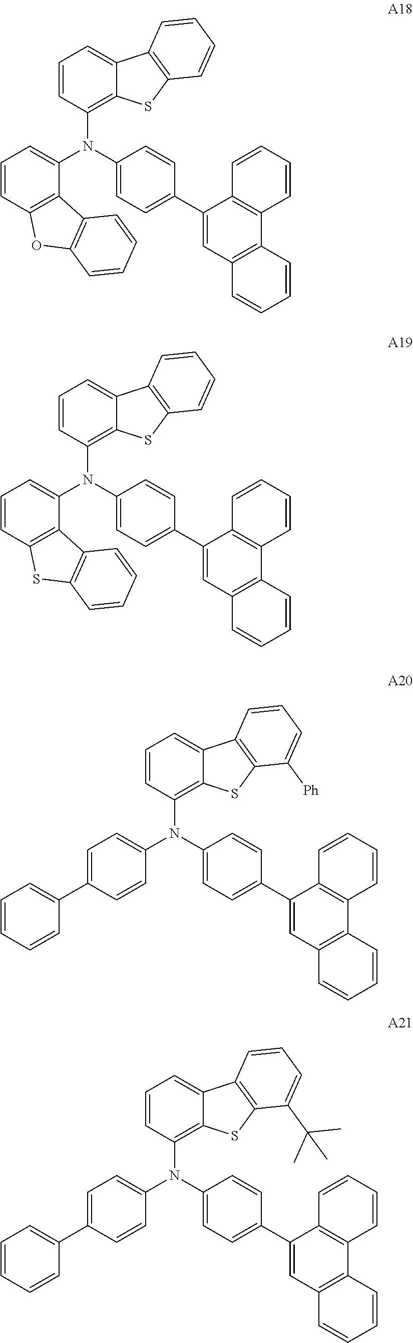 [SMILES: c1ccc2c(c1)cc(-c1ccc(N(c3cccc4c3sc3ccccc34)c3cccc4oc5ccccc5c34)cc1)c1ccccc12 ; c1ccc2c(c1)cc(-c1ccc(N(c3cccc4c3sc3ccccc34)c3cccc4sc5ccccc5c34)cc1)c1ccccc12 ; c1ccc(-c2ccc(N(c3ccc(-c4cc5ccccc5c5ccccc45)cc3)c3cccc4c3sc3c(-c5ccccc5)cccc34)cc2)cc1 ; CC(C)(C)c1cccc2c1sc1c(N(c3ccc(-c4ccccc4)cc3)c3ccc(-c4cc5ccccc5c5ccccc45)cc3)cccc12]

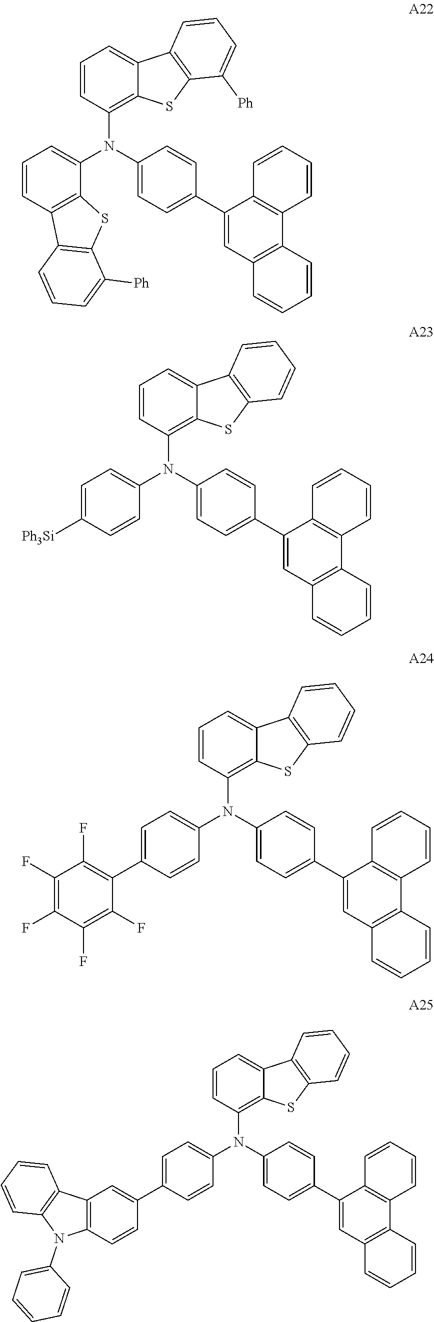

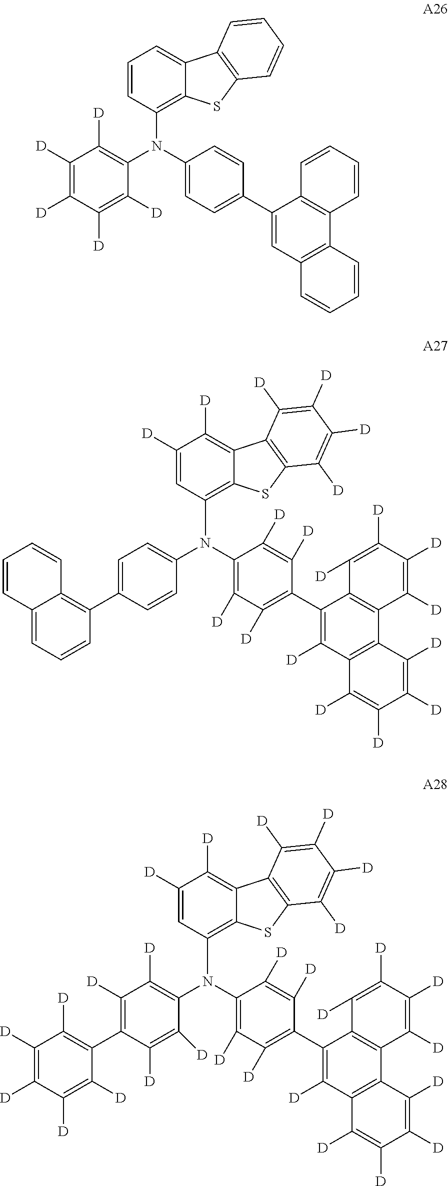

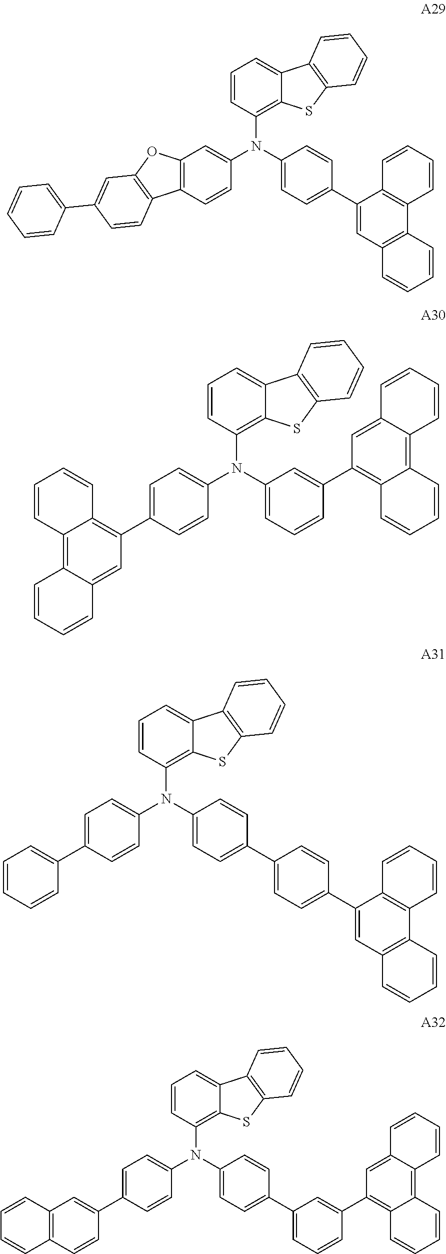 [SMILES: c1ccc(-c2ccc3c(c2)oc2cc(N(c4ccc(-c5cc6ccccc6c6ccccc56)cc4)c4cccc5c4sc4ccccc45)ccc23)cc1 ; c1cc(-c2cc3ccccc3c3ccccc23)cc(N(c2ccc(-c3cc4ccccc4c4ccccc34)cc2)c2cccc3c2sc2ccccc23)c1 ; c1ccc(-c2ccc(N(c3ccc(-c4ccc(-c5cc6ccccc6c6ccccc56)cc4)cc3)c3cccc4c3sc3ccccc34)cc2)cc1 ; c1cc(-c2ccc(N(c3ccc(-c4ccc5ccccc5c4)cc3)c3cccc4c3sc3ccccc34)cc2)cc(-c2cc3ccccc3c3ccccc23)c1]

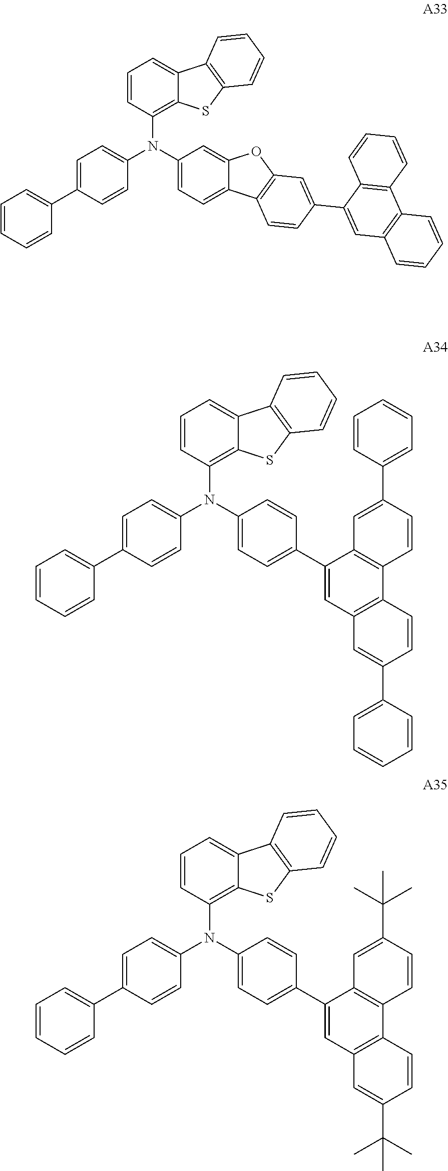 [SMILES: c1ccc(-c2ccc(N(c3ccc4c(c3)oc3cc(-c5cc6ccccc6c6ccccc56)ccc34)c3cccc4c3sc3ccccc34)cc2)cc1 ; c1ccc(-c2ccc(N(c3ccc(-c4cc5cc(-c6ccccc6)ccc5c5ccc(-c6ccccc6)cc45)cc3)c3cccc4c3sc3ccccc34)cc2)cc1 ; CC(C)(C)c1ccc2c(c1)cc(-c1ccc(N(c3ccc(-c4ccccc4)cc3)c3cccc4c3sc3ccccc34)cc1)c1cc(C(C)(C)C)ccc12]

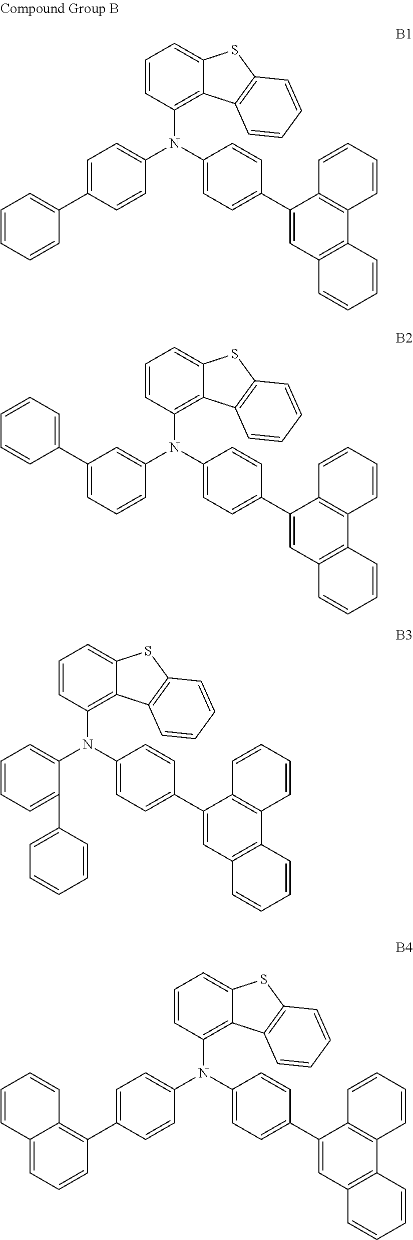 [SMILES: c1ccc(-c2ccc(N(c3ccc(-c4cc5ccccc5c5ccccc45)cc3)c3cccc4sc5ccccc5c34)cc2)cc1 ; c1ccc(-c2cccc(N(c3ccc(-c4cc5ccccc5c5ccccc45)cc3)c3cccc4sc5ccccc5c34)c2)cc1 ; c1ccc(-c2ccccc2N(c2ccc(-c3cc4ccccc4c4ccccc34)cc2)c2cccc3sc4ccccc4c23)cc1 ; c1ccc2c(-c3ccc(N(c4ccc(-c5cc6ccccc6c6ccccc56)cc4)c4cccc5sc6ccccc6c45)cc3)cccc2c1]

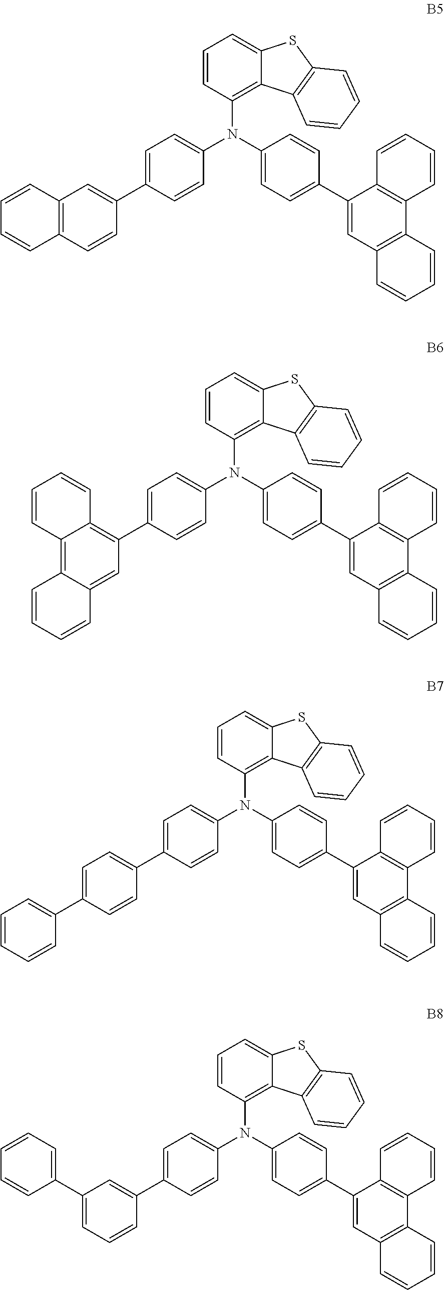 [SMILES: c1ccc2cc(-c3ccc(N(c4ccc(-c5cc6ccccc6c6ccccc56)cc4)c4cccc5sc6ccccc6c45)cc3)ccc2c1 ; c1ccc2c(c1)cc(-c1ccc(N(c3ccc(-c4cc5ccccc5c5ccccc45)cc3)c3cccc4sc5ccccc5c34)cc1)c1ccccc12 ; c1ccc(-c2ccc(-c3ccc(N(c4ccc(-c5cc6ccccc6c6ccccc56)cc4)c4cccc5sc6ccccc6c45)cc3)cc2)cc1 ; c1ccc(-c2cccc(-c3ccc(N(c4ccc(-c5cc6ccccc6c6ccccc56)cc4)c4cccc5sc6ccccc6c45)cc3)c2)cc1]

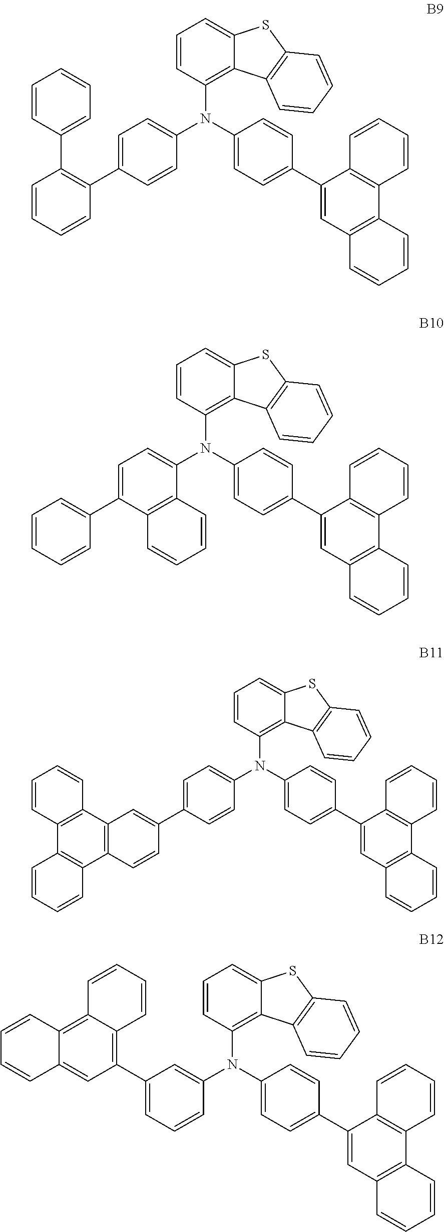 [SMILES: c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)c3ccccc23)cc1 ; c1ccc2c(c1)cc(-c1ccc(N(c3ccc(-c4ccc5c6ccccc6c6ccccc6c5c4)cc3)c3cccc4sc5ccccc5c34)cc1)c1ccccc12 ; c1cc(-c2cc3ccccc3c3ccccc23)cc(N(c2ccc(-c3cc4ccccc4c4ccccc34)cc2)c2cccc3sc4ccccc4c23)c1]

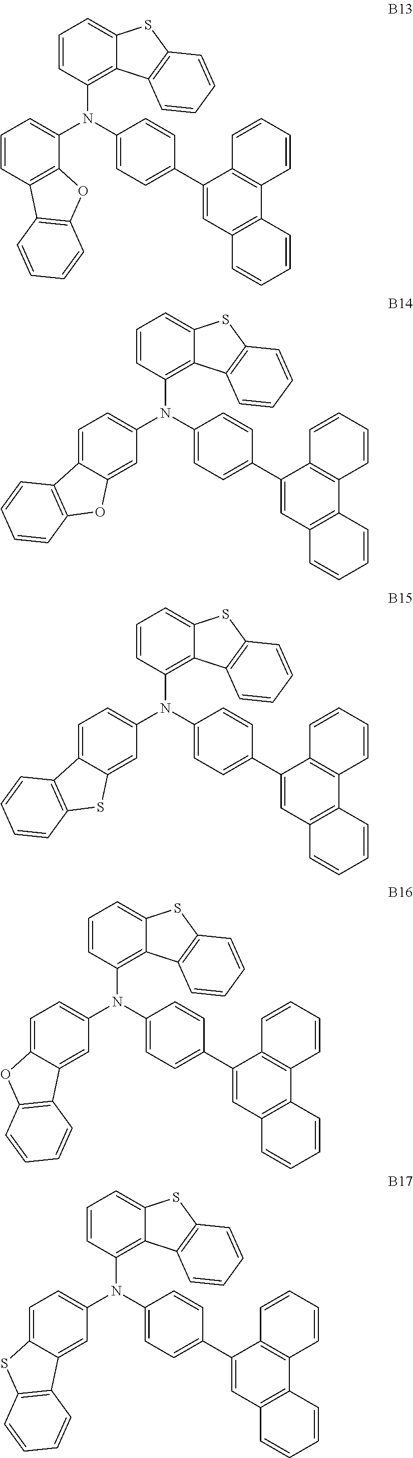 [SMILES: c1ccc2c(c1)cc(-c1ccc(N(c3cccc4c3oc3ccccc34)c3cccc4sc5ccccc5c34)cc1)c1ccccc12 ; c1ccc2c(c1)cc(-c1ccc(N(c3ccc4c(c3)oc3ccccc34)c3cccc4sc5ccccc5c34)cc1)c1ccccc12 ; c1ccc2c(c1)cc(-c1ccc(N(c3ccc4c(c3)sc3ccccc34)c3cccc4sc5ccccc5c34)cc1)c1ccccc12 ; c1ccc2c(c1)cc(-c1ccc(N(c3ccc4oc5ccccc5c4c3)c3cccc4sc5ccccc5c34)cc1)c1ccccc12 ; c1ccc2c(c1)cc(-c1ccc(N(c3ccc4sc5ccccc5c4c3)c3cccc4sc5ccccc5c34)cc1)c1ccccc12]

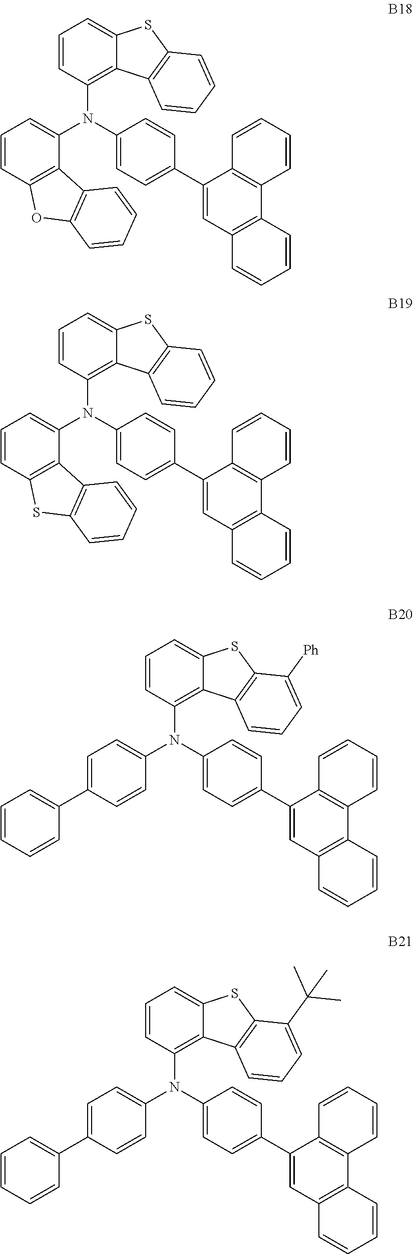 [SMILES: c1ccc2c(c1)cc(-c1ccc(N(c3cccc4oc5ccccc5c34)c3cccc4sc5ccccc5c34)cc1)c1ccccc12 ; c1ccc2c(c1)cc(-c1ccc(N(c3cccc4sc5ccccc5c34)c3cccc4sc5ccccc5c34)cc1)c1ccccc12 ; c1ccc(-c2ccc(N(c3ccc(-c4cc5ccccc5c5ccccc45)cc3)c3cccc4sc5c(-c6ccccc6)cccc5c34)cc2)cc1 ; CC(C)(C)c1cccc2c1sc1cccc(N(c3ccc(-c4ccccc4)cc3)c3ccc(-c4cc5ccccc5c5ccccc45)cc3)c12]

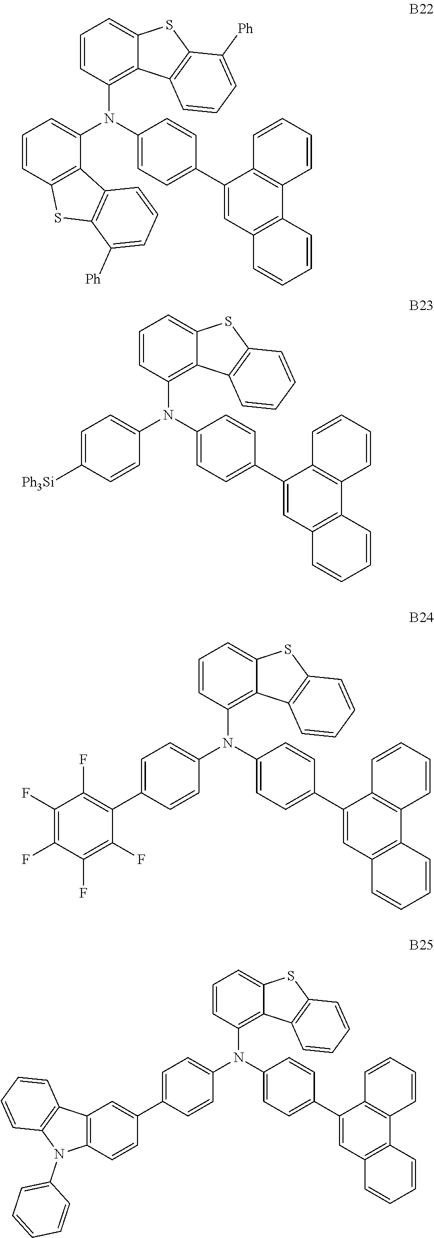

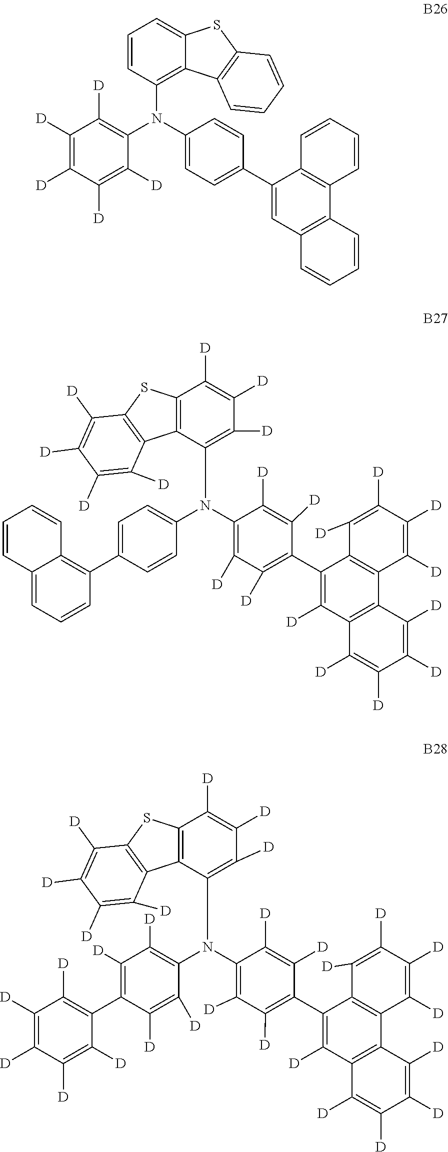

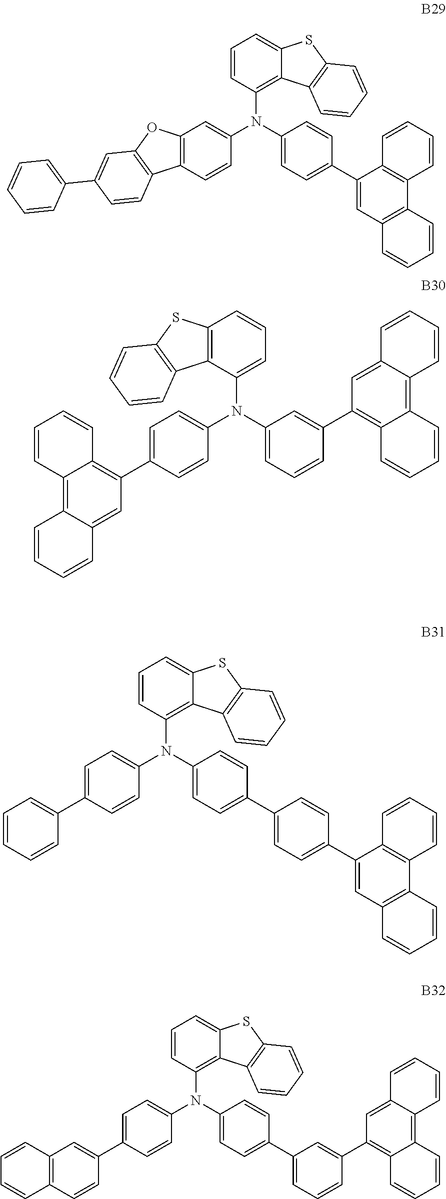 [SMILES: c1ccc(-c2ccc3c(c2)oc2cc(N(c4ccc(-c5cc6ccccc6c6ccccc56)cc4)c4cccc5sc6ccccc6c45)ccc23)cc1 ; c1cc(-c2cc3ccccc3c3ccccc23)cc(N(c2ccc(-c3cc4ccccc4c4ccccc34)cc2)c2cccc3sc4ccccc4c23)c1 ; c1ccc(-c2ccc(N(c3ccc(-c4ccc(-c5cc6ccccc6c6ccccc56)cc4)cc3)c3cccc4sc5ccccc5c34)cc2)cc1 ; c1cc(-c2ccc(N(c3ccc(-c4ccc5ccccc5c4)cc3)c3cccc4sc5ccccc5c34)cc2)cc(-c2cc3ccccc3c3ccccc23)c1]

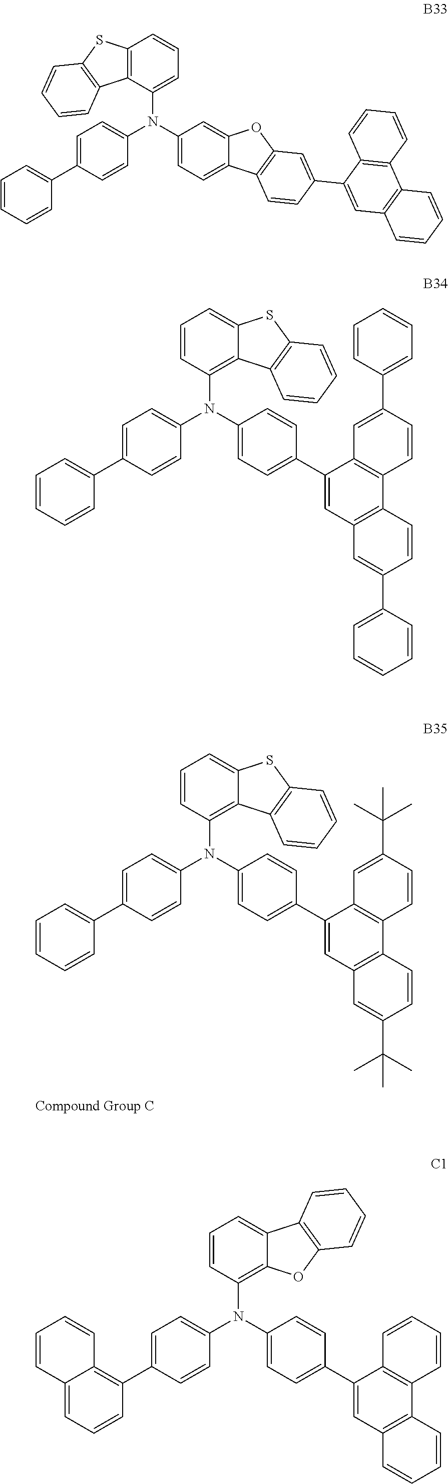 [SMILES: c1ccc(-c2ccc(N(c3ccc4c(c3)oc3cc(-c5cc6ccccc6c6ccccc56)ccc34)c3cccc4sc5ccccc5c34)cc2)cc1 ; c1ccc(-c2ccc(N(c3ccc(-c4cc5cc(-c6ccccc6)ccc5c5ccc(-c6ccccc6)cc45)cc3)c3cccc4sc5ccccc5c34)cc2)cc1 ; CC(C)(C)c1ccc2c(c1)cc(-c1ccc(N(c3ccc(-c4ccccc4)cc3)c3cccc4sc5ccccc5c34)cc1)c1cc(C(C)(C)C)ccc12 ; c1ccc2c(-c3ccc(N(c4ccc(-c5cc6ccccc6c6ccccc56)cc4)c4cccc5c4oc4ccccc45)cc3)cccc2c1]

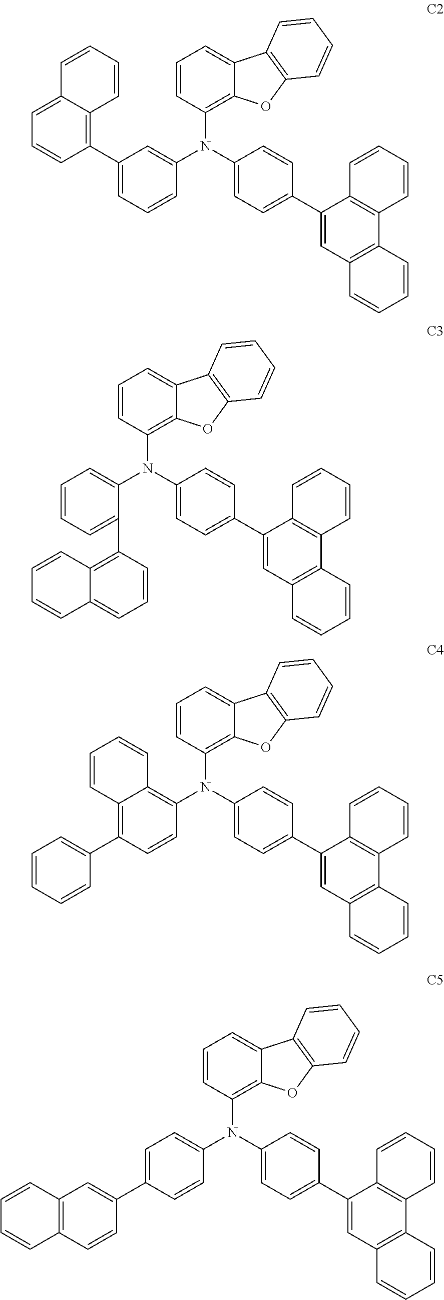

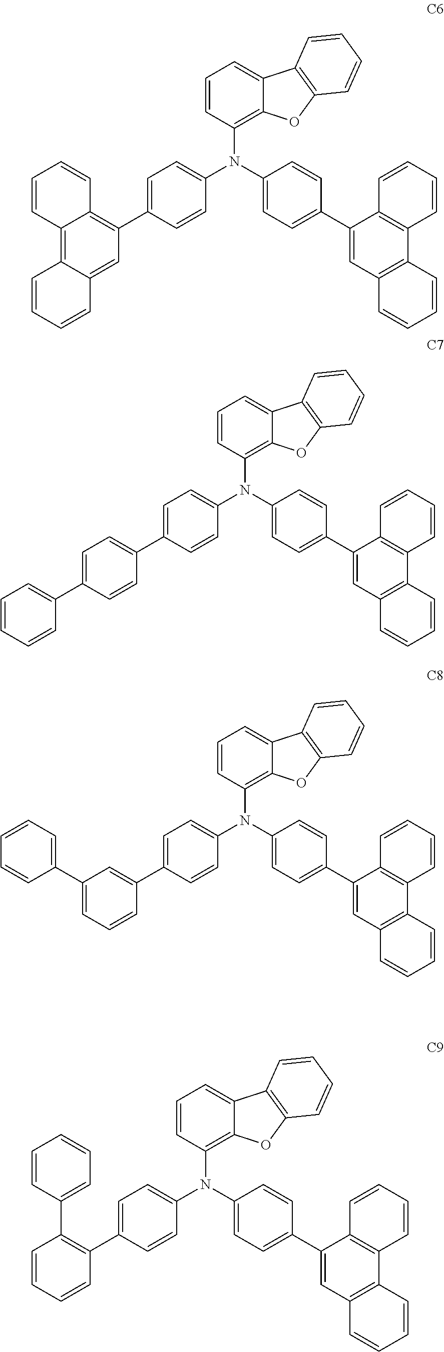 [SMILES: c1ccc2c(c1)cc(-c1ccc(N(c3ccc(-c4cc5ccccc5c5ccccc45)cc3)c3cccc4c3oc3ccccc34)cc1)c1ccccc12 ; c1ccc(-c2ccc(-c3ccc(N(c4ccc(-c5cc6ccccc6c6ccccc56)cc4)c4cccc5c4oc4ccccc45)cc3)cc2)cc1 ; c1ccc(-c2cccc(-c3ccc(N(c4ccc(-c5cc6ccccc6c6ccccc56)cc4)c4cccc5c4oc4ccccc45)cc3)c2)cc1 ; c1ccc(-c2ccccc2-c2ccc(N(c3ccc(-c4cc5ccccc5c5ccccc45)cc3)c3cccc4c3oc3ccccc34)cc2)cc1]

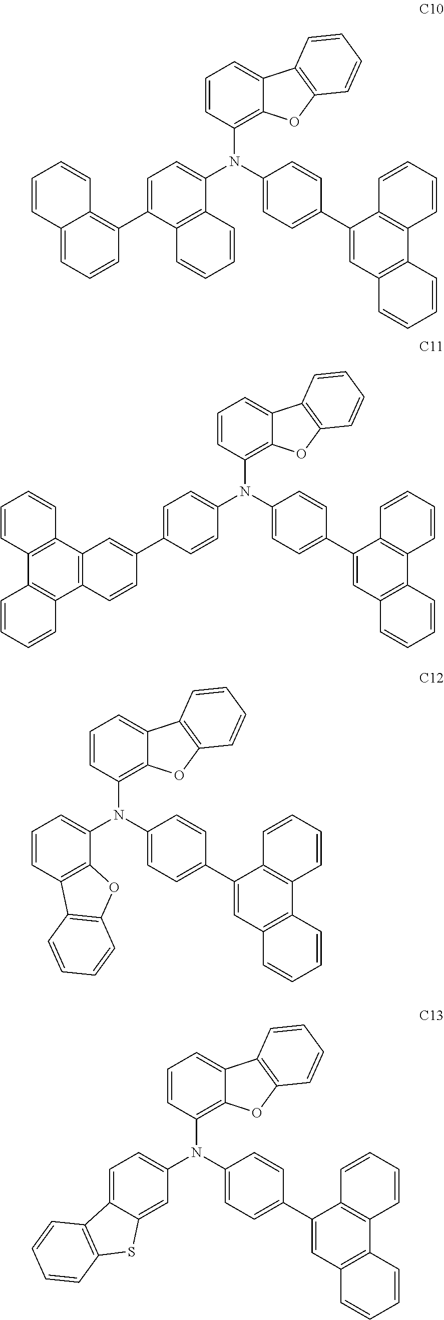

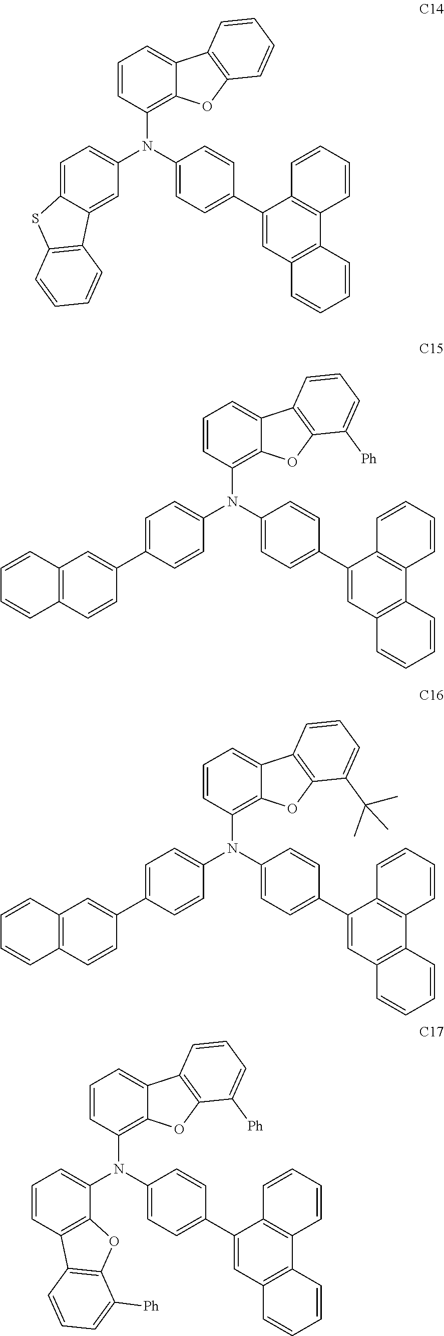 [SMILES: c1ccc2c(c1)cc(-c1ccc(N(c3ccc4sc5ccccc5c4c3)c3cccc4c3oc3ccccc34)cc1)c1ccccc12 ; c1ccc(-c2cccc3c2oc2c(N(c4ccc(-c5ccc6ccccc6c5)cc4)c4ccc(-c5cc6ccccc6c6ccccc56)cc4)cccc23)cc1 ; CC(C)(C)c1cccc2c1oc1c(N(c3ccc(-c4ccc5ccccc5c4)cc3)c3ccc(-c4cc5ccccc5c5ccccc45)cc3)cccc12 ; c1ccc(-c2cccc3c2oc2c(N(c4ccc(-c5cc6ccccc6c6ccccc56)cc4)c4cccc5c4oc4c(-c6ccccc6)cccc45)cccc23)cc1]

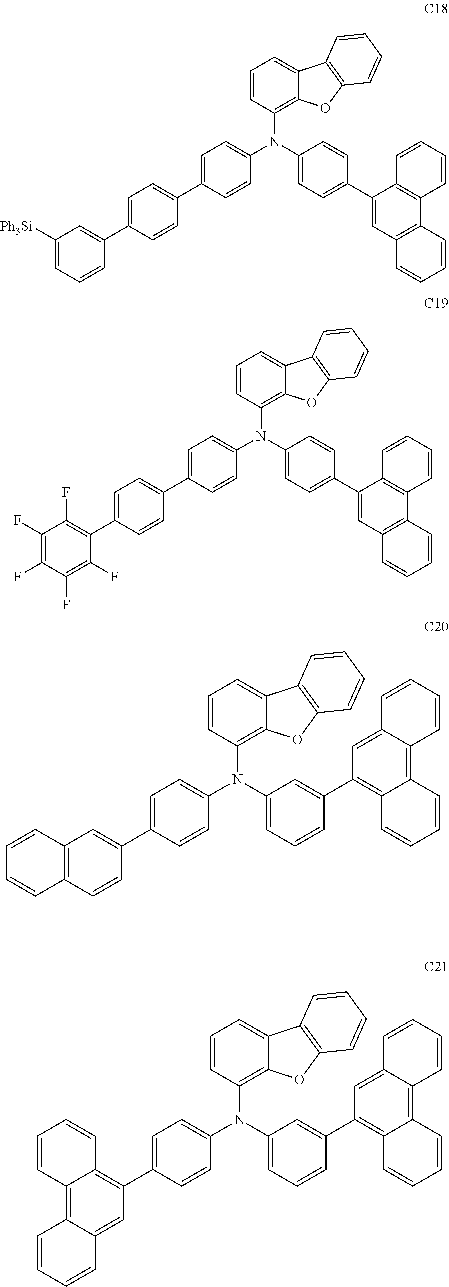

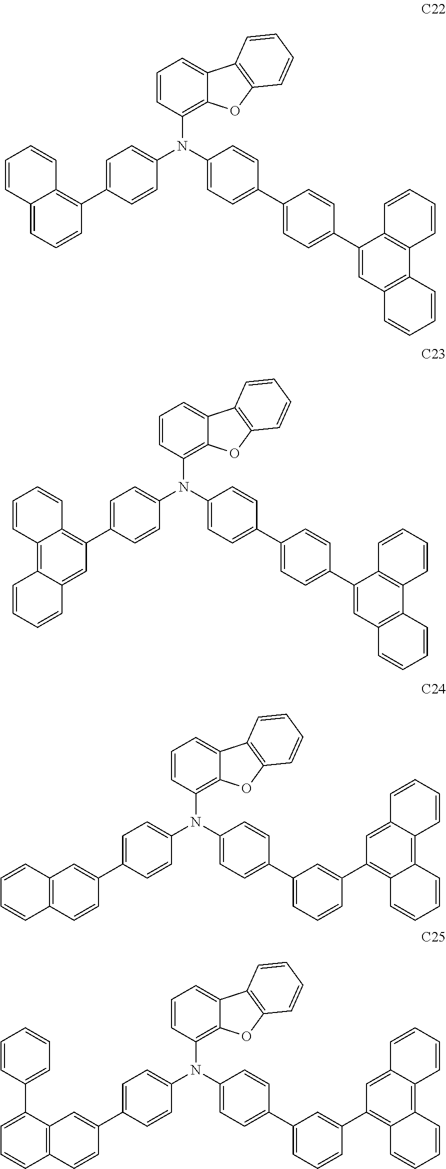 [SMILES: c1ccc2c(-c3ccc(N(c4ccc(-c5ccc(-c6cc7ccccc7c7ccccc67)cc5)cc4)c4cccc5c4oc4ccccc45)cc3)cccc2c1 ; c1ccc2c(c1)cc(-c1ccc(-c3ccc(N(c4ccc(-c5cc6ccccc6c6ccccc56)cc4)c4cccc5c4oc4ccccc45)cc3)cc1)c1ccccc12 ; c1cc(-c2ccc(N(c3ccc(-c4ccc5ccccc5c4)cc3)c3cccc4c3oc3ccccc34)cc2)cc(-c2cc3ccccc3c3ccccc23)c1 ; c1ccc(-c2cccc3ccc(-c4ccc(N(c5ccc(-c6cccc(-c7cc8ccccc8c8ccccc78)c6)cc5)c5cccc6c5oc5ccccc56)cc4)cc23)cc1]

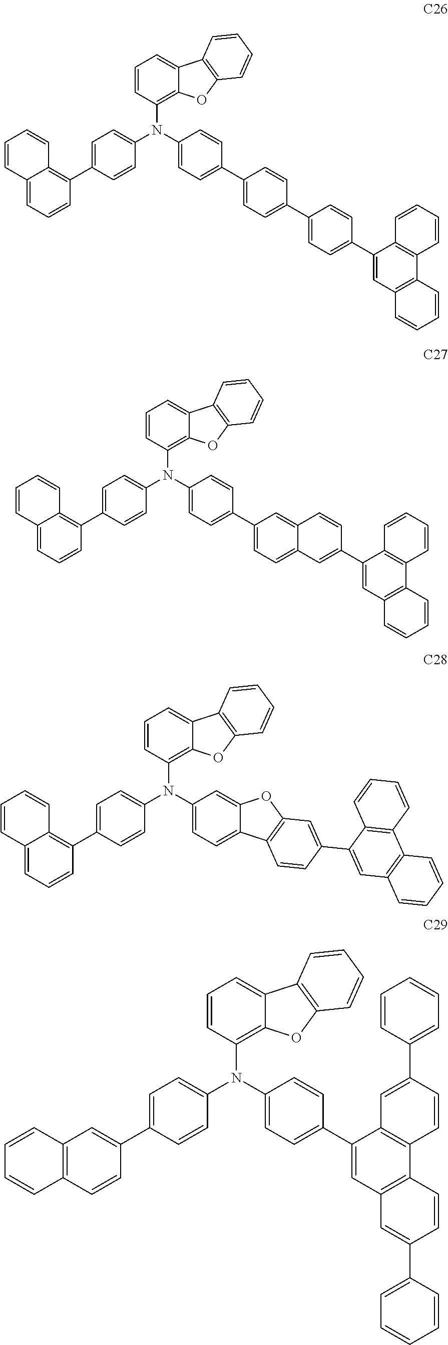 [SMILES: c1ccc2c(-c3ccc(N(c4ccc(-c5ccc(-c6ccc(-c7cc8ccccc8c8ccccc78)cc6)cc5)cc4)c4cccc5c4oc4ccccc45)cc3)cccc2c1 ; c1ccc2c(-c3ccc(N(c4ccc(-c5ccc6cc(-c7cc8ccccc8c8ccccc78)ccc6c5)cc4)c4cccc5c4oc4ccccc45)cc3)cccc2c1 ; c1ccc2c(-c3ccc(N(c4ccc5c(c4)oc4cc(-c6cc7ccccc7c7ccccc67)ccc45)c4cccc5c4oc4ccccc45)cc3)cccc2c1 ; c1ccc(-c2ccc3c(c2)cc(-c2ccc(N(c4ccc(-c5ccc6ccccc6c5)cc4)c4cccc5c4oc4ccccc45)cc2)c2cc(-c4ccccc4)ccc23)cc1]

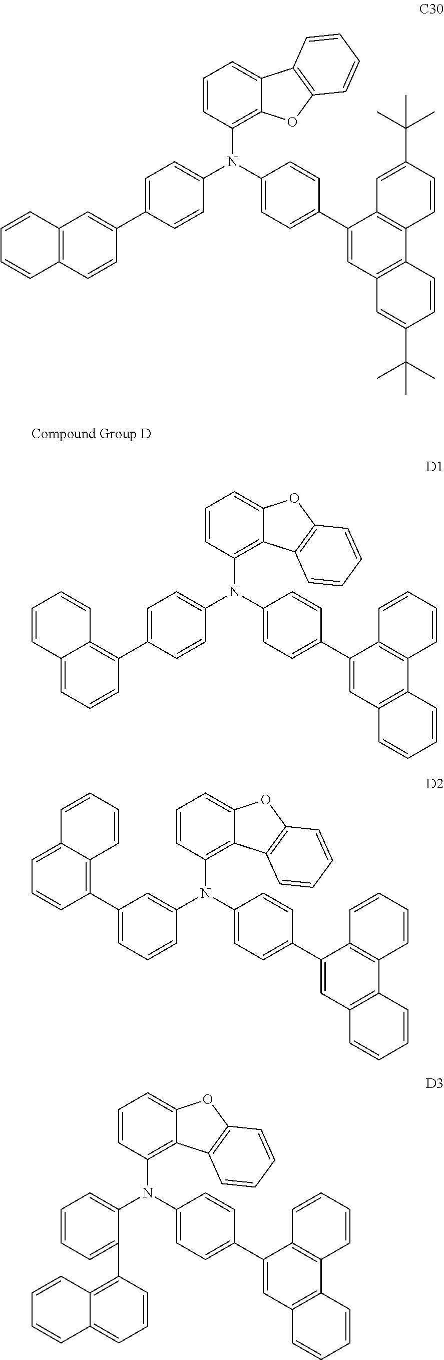 [SMILES: CC(C)(C)c1ccc2c(c1)cc(-c1ccc(N(c3ccc(-c4ccc5ccccc5c4)cc3)c3cccc4c3oc3ccccc34)cc1)c1cc(C(C)(C)C)ccc12 ; c1ccc2c(-c3ccc(N(c4ccc(-c5cc6ccccc6c6ccccc56)cc4)c4cccc5oc6ccccc6c45)cc3)cccc2c1 ; c1cc(-c2cccc3ccccc23)cc(N(c2ccc(-c3cc4ccccc4c4ccccc34)cc2)c2cccc3oc4ccccc4c23)c1 ; c1ccc(N(c2ccc(-c3cc4ccccc4c4ccccc34)cc2)c2cccc3oc4ccccc4c23)c(-c2cccc3ccccc23)c1]

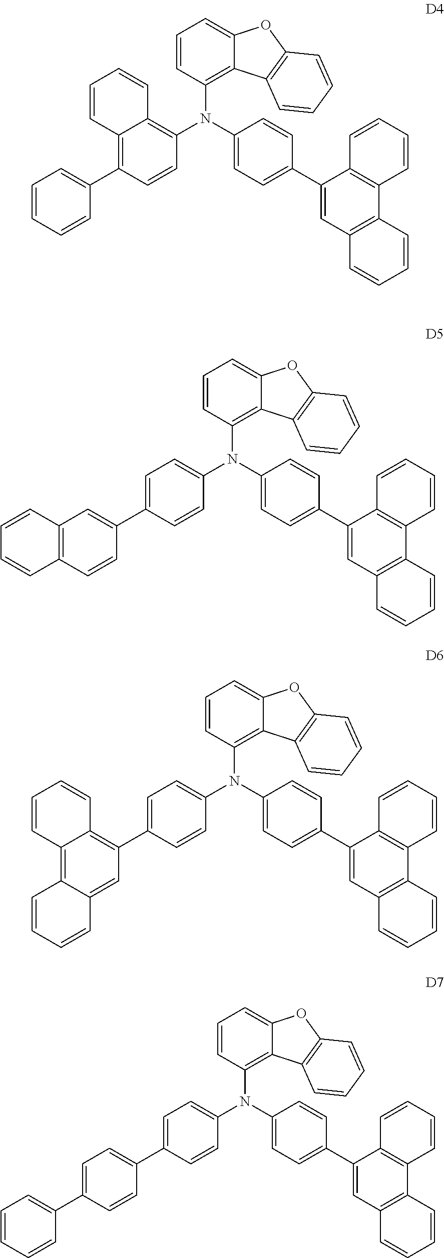 [SMILES: c1ccc(-c2ccc(N(c3ccc(-c4cc5ccccc5c5ccccc45)cc3)c3cccc4oc5ccccc5c34)c3ccccc23)cc1 ; c1ccc2cc(-c3ccc(N(c4ccc(-c5cc6ccccc6c6ccccc56)cc4)c4cccc5oc6ccccc6c45)cc3)ccc2c1 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc(-c2ccc(-c3ccc(N(c4ccc(-c5cc6ccccc6c6ccccc56)cc4)c4cccc5oc6ccccc6c45)cc3)cc2)cc1]

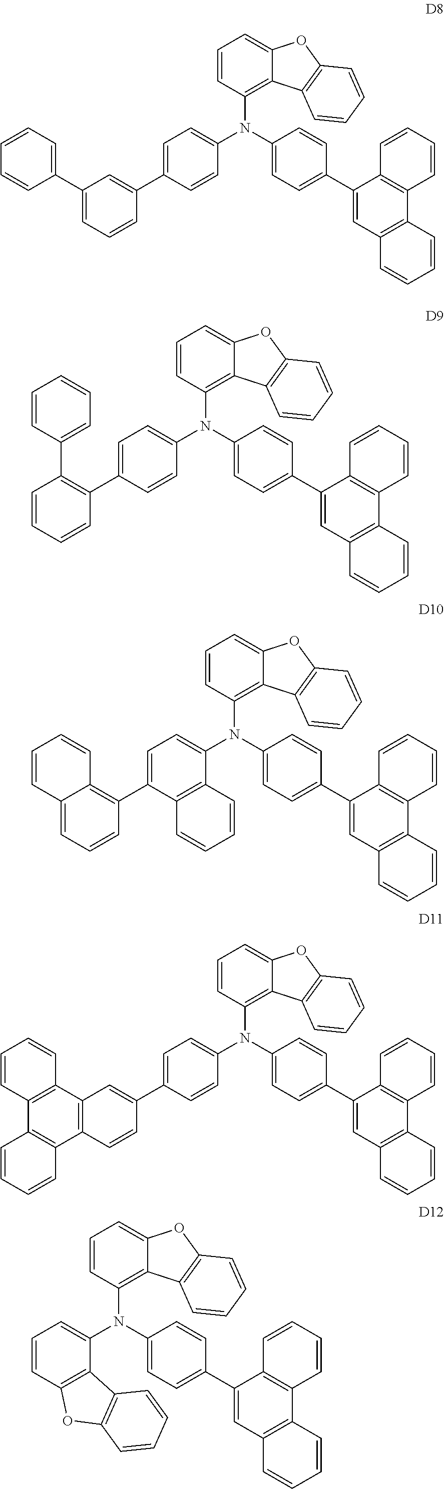 [SMILES: c1ccc(-c2cccc(-c3ccc(N(c4ccc(-c5cc6ccccc6c6ccccc56)cc4)c4cccc5oc6ccccc6c45)cc3)c2)cc1 ; c1ccc(-c2ccccc2-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)c4ccccc34)cccc2c1 ; c1ccc2c(c1)cc(-c1ccc(N(c3ccc(-c4ccc5c6ccccc6c6ccccc6c5c4)cc3)c3cccc4oc5ccccc5c34)cc1)c1ccccc12 ; c1ccc2c(c1)cc(-c1ccc(N(c3cccc4oc5ccccc5c34)c3cccc4oc5ccccc5c34)cc1)c1ccccc12]

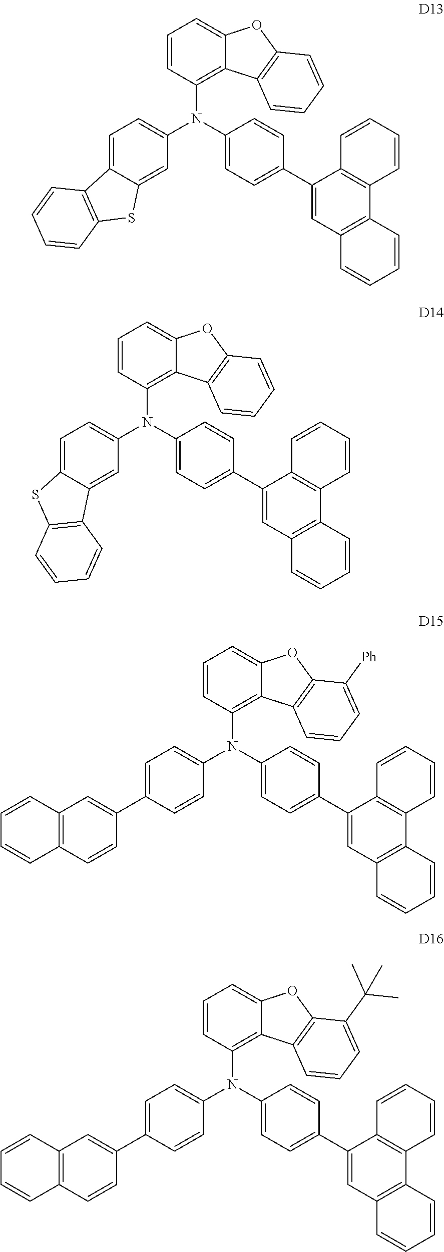 [SMILES: c1ccc2c(c1)cc(-c1ccc(N(c3ccc4c(c3)sc3ccccc34)c3cccc4oc5ccccc5c34)cc1)c1ccccc12 ; c1ccc2c(c1)cc(-c1ccc(N(c3ccc4sc5ccccc5c4c3)c3cccc4oc5ccccc5c34)cc1)c1ccccc12 ; c1ccc(-c2cccc3c2oc2cccc(N(c4ccc(-c5ccc6ccccc6c5)cc4)c4ccc(-c5cc6ccccc6c6ccccc56)cc4)c23)cc1 ; CC(C)(C)c1cccc2c1oc1cccc(N(c3ccc(-c4ccc5ccccc5c4)cc3)c3ccc(-c4cc5ccccc5c5ccccc45)cc3)c12]

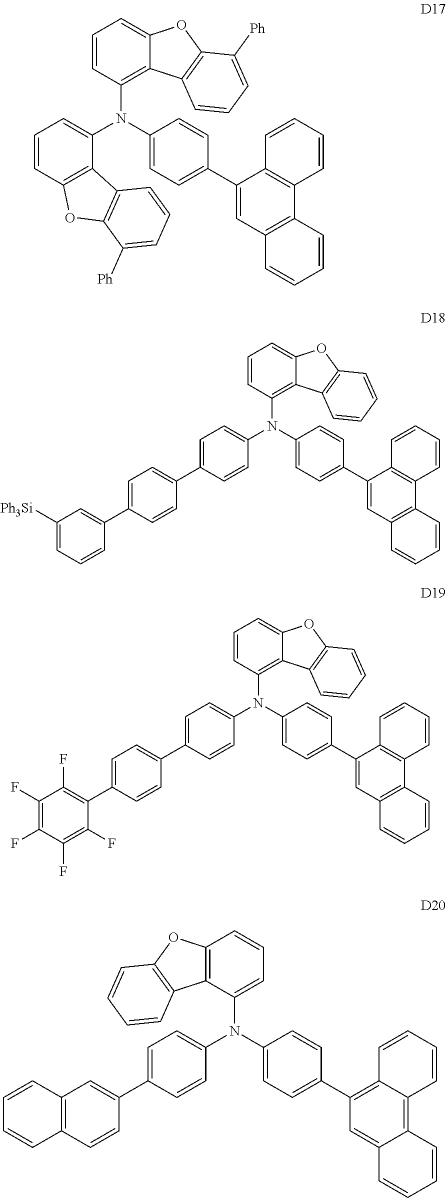

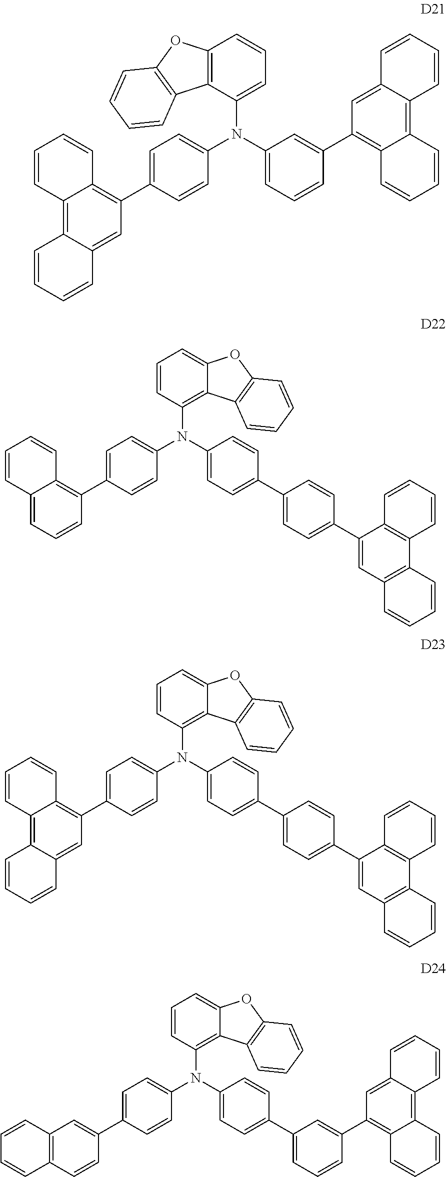 [SMILES: c1cc(-c2cc3ccccc3c3ccccc23)cc(N(c2ccc(-c3cc4ccccc4c4ccccc34)cc2)c2cccc3oc4ccccc4c23)c1 ; c1ccc2c(-c3ccc(N(c4ccc(-c5ccc(-c6cc7ccccc7c7ccccc67)cc5)cc4)c4cccc5oc6ccccc6c45)cc3)cccc2c1 ; c1ccc2c(c1)cc(-c1ccc(-c3ccc(N(c4ccc(-c5cc6ccccc6c6ccccc56)cc4)c4cccc5oc6ccccc6c45)cc3)cc1)c1ccccc12 ; c1cc(-c2ccc(N(c3ccc(-c4ccc5ccccc5c4)cc3)c3cccc4oc5ccccc5c34)cc2)cc(-c2cc3ccccc3c3ccccc23)c1]

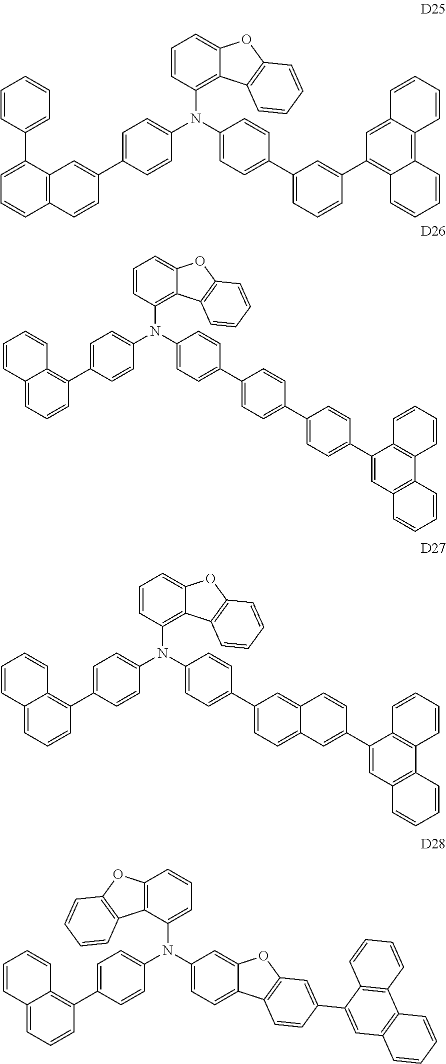 [SMILES: c1ccc(-c2cccc3ccc(-c4ccc(N(c5ccc(-c6cccc(-c7cc8ccccc8c8ccccc78)c6)cc5)c5cccc6oc7ccccc7c56)cc4)cc23)cc1 ; c1ccc2c(-c3ccc(N(c4ccc(-c5ccc(-c6ccc(-c7cc8ccccc8c8ccccc78)cc6)cc5)cc4)c4cccc5oc6ccccc6c45)cc3)cccc2c1 ; c1ccc2c(-c3ccc(N(c4ccc(-c5ccc6cc(-c7cc8ccccc8c8ccccc78)ccc6c5)cc4)c4cccc5oc6ccccc6c45)cc3)cccc2c1 ; c1ccc2c(-c3ccc(N(c4ccc5c(c4)oc4cc(-c6cc7ccccc7c7ccccc67)ccc45)c4cccc5oc6ccccc6c45)cc3)cccc2c1]

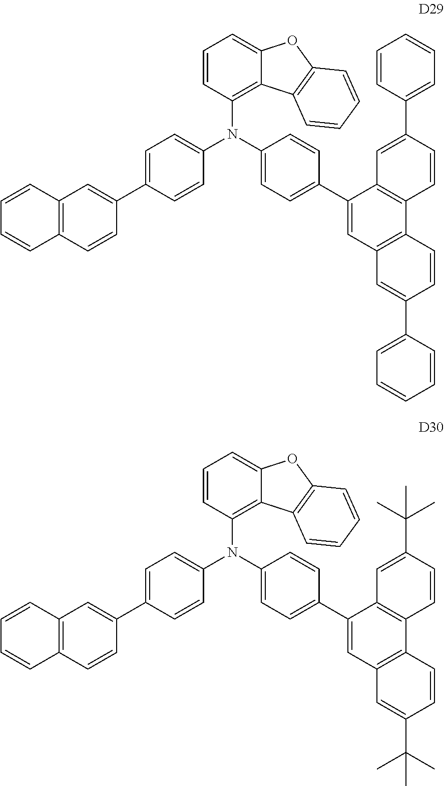 [SMILES: c1ccc(-c2ccc3c(c2)cc(-c2ccc(N(c4ccc(-c5ccc6ccccc6c5)cc4)c4cccc5oc6ccccc6c45)cc2)c2cc(-c4ccccc4)ccc23)cc1 ; CC(C)(C)c1ccc2c(c1)cc(-c1ccc(N(c3ccc(-c4ccc5ccccc5c4)cc3)c3cccc4oc5ccccc5c34)cc1)c1cc(C(C)(C)C)ccc12]

12. An amine compound of the following Formula 1 for use in a luminescence device: 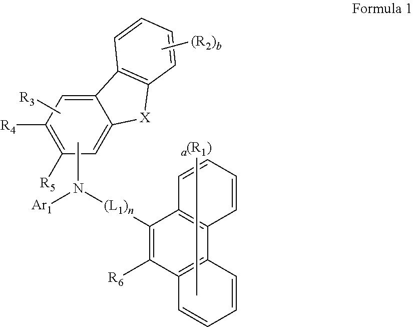
in Formula 1,
X is O or S;
R₁ to R₅ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
R₆ is a hydrogen atom- or a deuterium atom;
L₁ is a substituted or unsubstituted arylene group of 6 to 40 carbon atoms for forming a ring, or a substituted or unsubstituted heteroarylene group of 2 to 40 carbon atoms for forming a ring;
Ar₁ is a substituted or unsubstituted aryl group of 6 to 40 carbon atoms for forming a ring, or a substituted or unsubstituted heteroaryl group of 2 to 12 carbon atoms for forming a ring;
if X is O, and Ar₁ is a substituted or unsubstituted aryl group with 16 to 40 carbon atoms forming a ring;
a is an integer of 0 to 8;
b is an integer of 0 to 4; and
n is an integer of 1 to 3.

13. The amine compound of claim 12, wherein Ar₁ is a group of the following Formula 2: [symbol]
ₚ(Ar₁₁)—Ar₁₂ Formula 2
in Formula 2,
Ar₁₁ is a substituted or unsubstituted arylene group of 6 to 40 carbon atoms for forming a ring, or a substituted or unsubstituted heteroarylene group of 2 to 12 carbon atoms for forming a ring;
Ar₁₂ is a substituted or unsubstituted aryl group of 6 to 40 carbon atoms for forming a ring, or a substituted or unsubstituted heteroaryl group of 2 to 12 carbon atoms for forming a ring; and
p is 0 or 1.

14. The amine compound of claim 12, wherein Ar₁ is a group of Formulae 1-1 to 1-10: 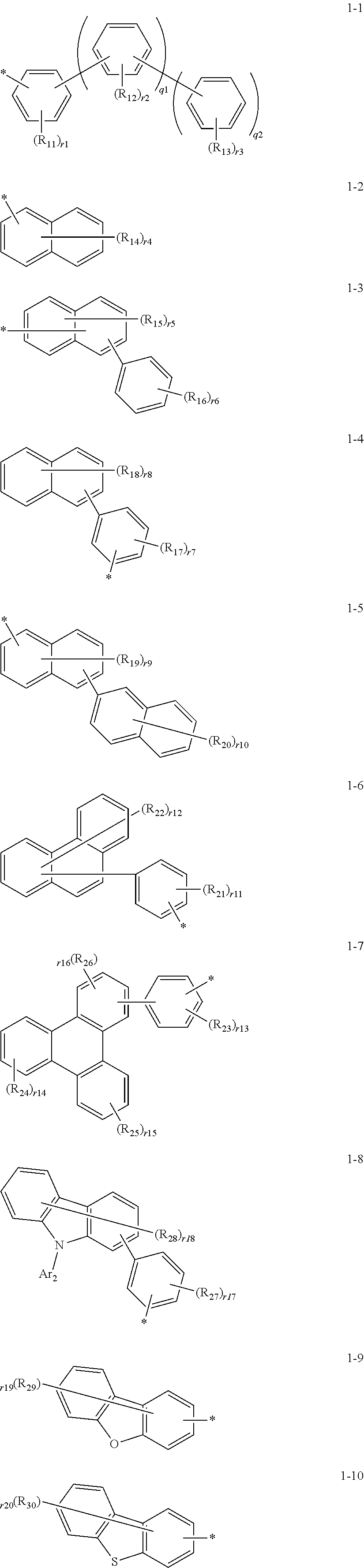
in Formulae 1-1 to 1-10,
R₁₁ to R₃₀ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
Ar₂ is a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
q1 and q2 are each, independently from one another, 0 or 1;
r1, r7, r11, and r13 to r17 are each, independently from one another, an integer of 0 to 4;
r2, r3, and r6 are each, independently from one another, an integer of 0 to 5;
r4, r8, r10, and r18 to r20 are each, independently from one another, an integer of 0 to 7;
r5, and r9 are each, independently from one another, an integer of 0 to 6; and
r12 is an integer of 0 to 9.

15. The amine compound of claim 12, wherein Ar₁ is represented by a group of Formulae 1-11 to 1-20: 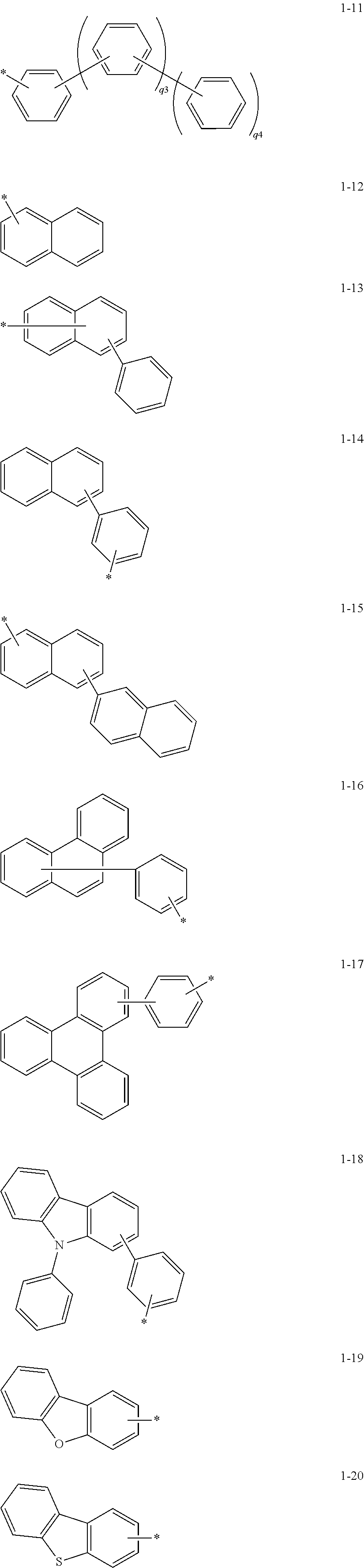
in 1-11,
q3 and q4 are each, independently from one another, 0 or 1.

16. The amine compound of claim 12, wherein L₁ is a group of Formulae 2-1 to 2-4: 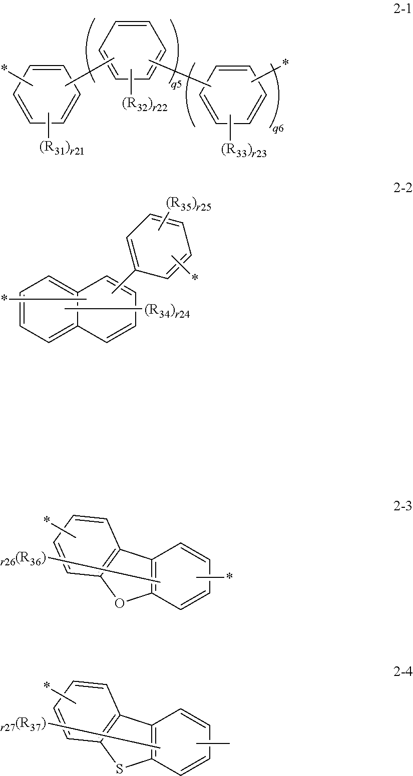
in Formulae 2-1 to 2-4,
R₃₁ to R₃₇ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
q5 and q6 are each, independently from one another, 0 or 1;
r21 to r23, and r25 are each, independently from one another, an integer of 0 to 4; and
r24, r26, and r27 are each, independently from one another, an integer of 0 to 6.

17. The amine compound of claim 12, wherein L₁ is a group of Formulae 2-11 to 2-14: 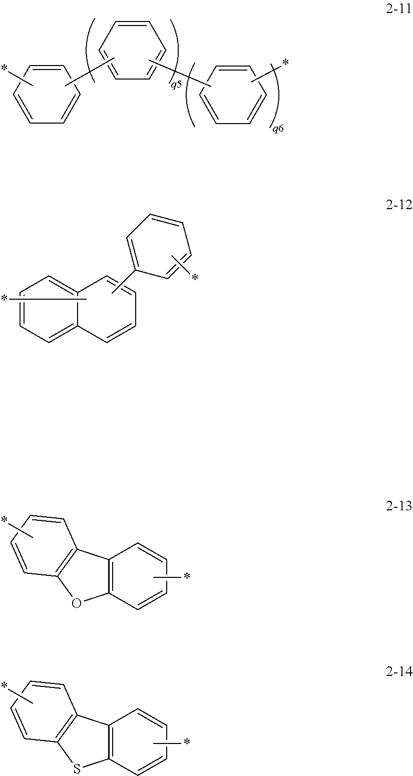
in Formulae 2-11,
q5 and q6 are each, independently from one another, 0 or 1.

18. The amine compound of claim 12, wherein the amine compound of Formula 1 is a compound of the following Formula 1-1: 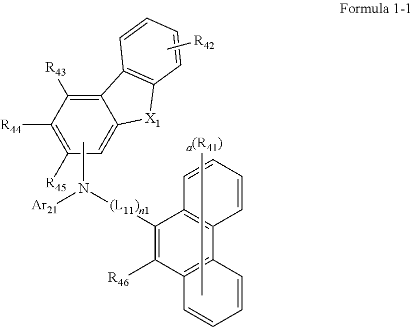
in Formula 1-1,
R₄₁ and R₄₂ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl group of 6 to 40 carbon atoms for forming a ring, or a substituted or unsubstituted heteroaryl group of 2 to 40 carbon atoms for forming a ring;
R₄₃ to R₄₆ are each, independently from one another, a hydrogen atom, or a deuterium atom;
L₁₁ is a substituted or unsubstituted arylene group of 6 to 40 carbon atoms for forming a ring, or a substituted or unsubstituted heteroarylene group of 2 to 40 carbon atoms for forming a ring;
Ar₂₁ is a substituted or unsubstituted aryl group of 6 to 40 carbon atoms for forming a ring, or a substituted or unsubstituted heteroaryl group of 2 to 12 carbon atoms for forming a ring;
if X₁ is O, and Ar₂₁ is a substituted or unsubstituted aryl group with 16 to 40 carbon atoms forming a ring;
a1 is an integer of 0 to 8; and
n1 is an integer of 1 to 3.

19. The amine compound of claim 12, wherein the amine compound of Formula 1 is a compound from the following Formula 1-2: 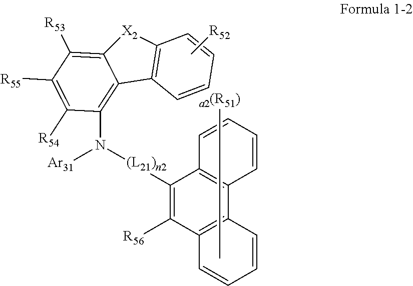
in Formula 1-2,
R₅₁ and R₅₂ are each, independently from one another, a hydrogen atom, a deuterium atom, a halogen atom, a substituted or unsubstituted silyl group, a substituted or unsubstituted alkyl group of 1 to 20 carbon atoms, a substituted or unsubstituted aryl amine group of 6 to 40 carbon atoms for forming a ring, or a substituted or unsubstituted heteroaryl group of 2 to 40 carbon atoms for forming a ring;
R₅₃ to R₅₆ are each, independently from one another, a hydrogen atom, or a deuterium atom;
L₂₁ is a substituted or unsubstituted arylene group of 6 to 40 carbon atoms for forming a ring, or a substituted or unsubstituted heteroarylene group of 2 to 40 carbon atoms for forming a ring;
Ar₃₁ is a substituted or unsubstituted aryl group of 6 to 40 carbon atoms for forming a ring, or a substituted or unsubstituted heteroaryl group of 2 to 12 carbon atoms for forming a ring;
if X₂ is O, and Ar₃₁ is a substituted or unsubstituted aryl group with 16 to 40 carbon atoms forming the ring;
a2 is an integer of 0 to 8; and
n2 is an integer of 1 to 3.

20. The amine compound of claim 12, wherein the amine compound of Formula 1 comprises at least one compound from Compound Groups A-D: 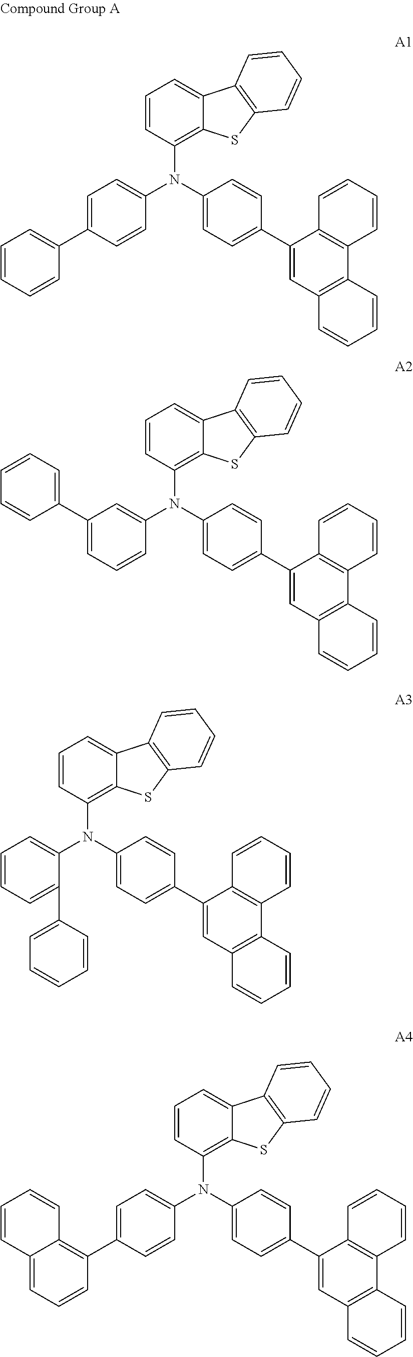 [SMILES: c1ccc(-c2ccc(N(c3ccc(-c4cc5ccccc5c5ccccc45)cc3)c3cccc4c3sc3ccccc34)cc2)cc1 ; c1ccc(-c2cccc(N(c3ccc(-c4cc5ccccc5c5ccccc45)cc3)c3cccc4c3sc3ccccc34)c2)cc1 ; c1ccc(-c2ccccc2N(c2ccc(-c3cc4ccccc4c4ccccc34)cc2)c2cccc3c2sc2ccccc23)cc1 ; c1ccc2c(-c3ccc(N(c4ccc(-c5cc6ccccc6c6ccccc56)cc4)c4cccc5c4sc4ccccc45)cc3)cccc2c1] 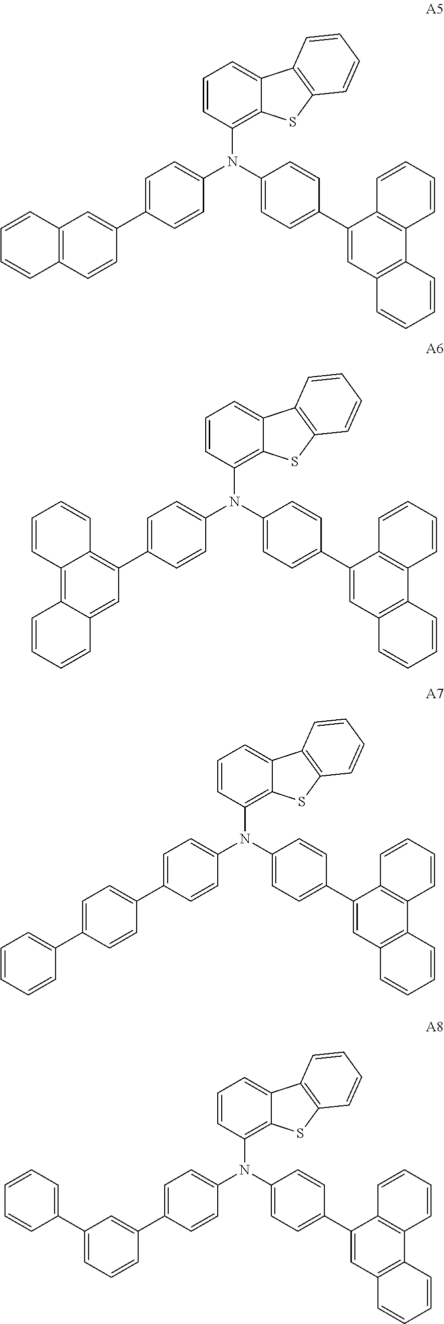 [SMILES: c1ccc2cc(-c3ccc(N(c4ccc(-c5cc6ccccc6c6ccccc56)cc4)c4cccc5c4sc4ccccc45)cc3)ccc2c1 ; c1ccc2c(c1)cc(-c1ccc(N(c3ccc(-c4cc5ccccc5c5ccccc45)cc3)c3cccc4c3sc3ccccc34)cc1)c1ccccc12 ; c1ccc(-c2ccc(-c3ccc(N(c4ccc(-c5cc6ccccc6c6ccccc56)cc4)c4cccc5c4sc4ccccc45)cc3)cc2)cc1 ; c1ccc(-c2cccc(-c3ccc(N(c4ccc(-c5cc6ccccc6c6ccccc56)cc4)c4cccc5c4sc4ccccc45)cc3)c2)cc1] 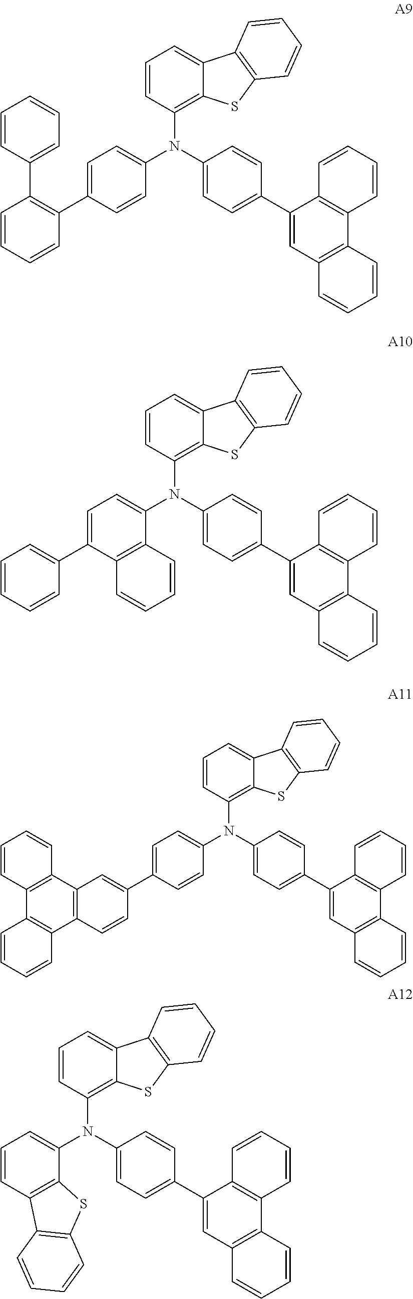 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)c3ccccc23)cc1 ; c1ccc2c(c1)cc(-c1ccc(N(c3ccc(-c4ccc5c6ccccc6c6ccccc6c5c4)cc3)c3cccc4c3sc3ccccc34)cc1)c1ccccc12 ; c1ccc2c(c1)cc(-c1ccc(N(c3cccc4c3sc3ccccc34)c3cccc4c3sc3ccccc34)cc1)c1ccccc12] 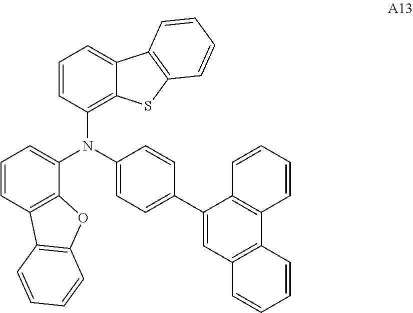 [SMILES: c1ccc2c(c1)cc(-c1ccc(N(c3cccc4c3oc3ccccc34)c3cccc4c3sc3ccccc34)cc1)c1ccccc12] 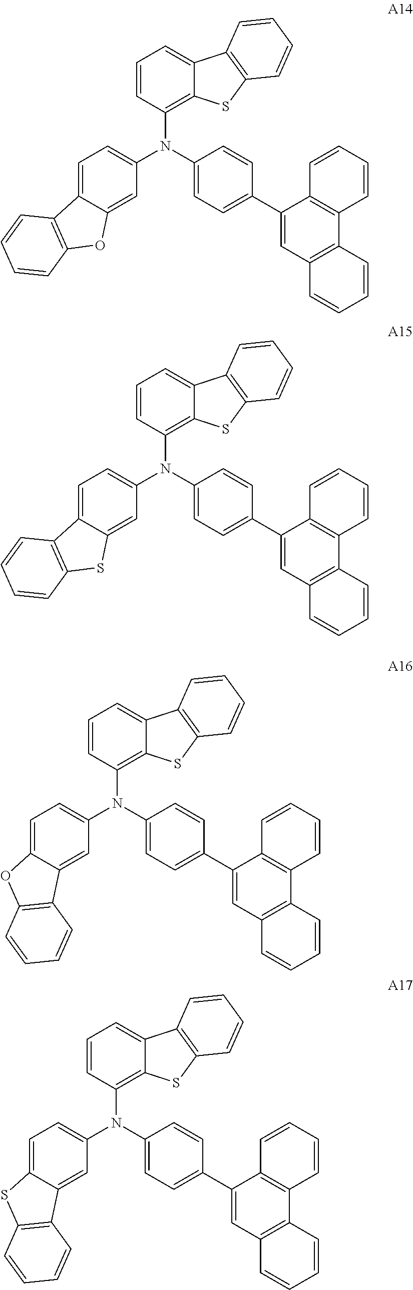 [SMILES: c1ccc2c(c1)cc(-c1ccc(N(c3ccc4c(c3)oc3ccccc34)c3cccc4c3sc3ccccc34)cc1)c1ccccc12 ; c1ccc2c(c1)cc(-c1ccc(N(c3ccc4c(c3)sc3ccccc34)c3cccc4c3sc3ccccc34)cc1)c1ccccc12 ; c1ccc2c(c1)cc(-c1ccc(N(c3ccc4oc5ccccc5c4c3)c3cccc4c3sc3ccccc34)cc1)c1ccccc12 ; c1ccc2c(c1)cc(-c1ccc(N(c3ccc4sc5ccccc5c4c3)c3cccc4c3sc3ccccc34)cc1)c1ccccc12] 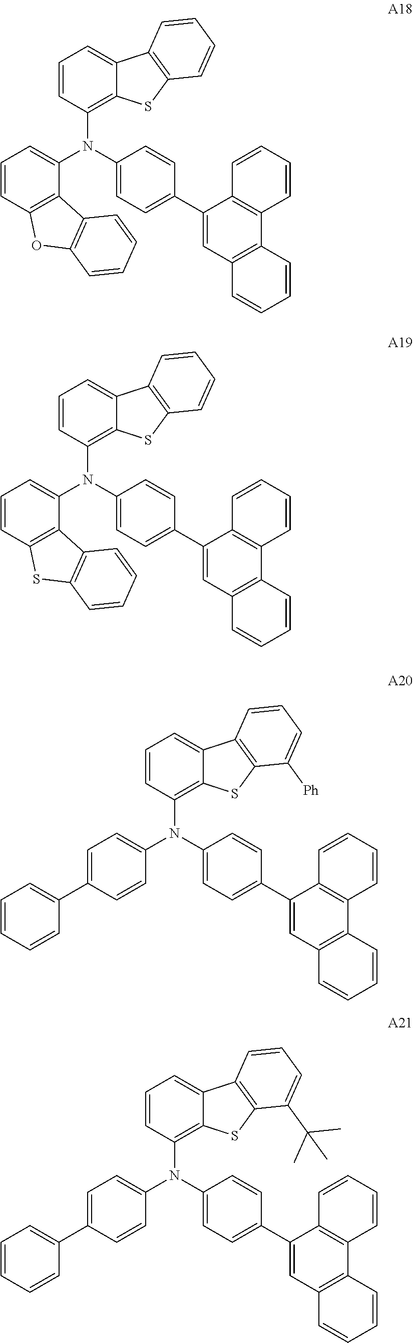 [SMILES: c1ccc2c(c1)cc(-c1ccc(N(c3cccc4c3sc3ccccc34)c3cccc4oc5ccccc5c34)cc1)c1ccccc12 ; c1ccc2c(c1)cc(-c1ccc(N(c3cccc4c3sc3ccccc34)c3cccc4sc5ccccc5c34)cc1)c1ccccc12 ; c1ccc(-c2ccc(N(c3ccc(-c4cc5ccccc5c5ccccc45)cc3)c3cccc4c3sc3c(-c5ccccc5)cccc34)cc2)cc1 ; CC(C)(C)c1cccc2c1sc1c(N(c3ccc(-c4ccccc4)cc3)c3ccc(-c4cc5ccccc5c5ccccc45)cc3)cccc12] 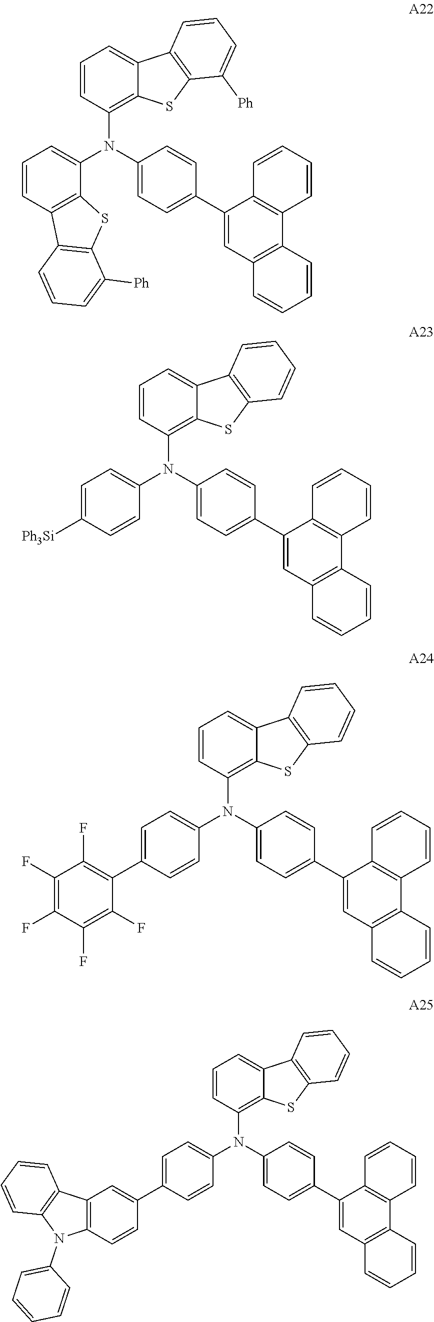 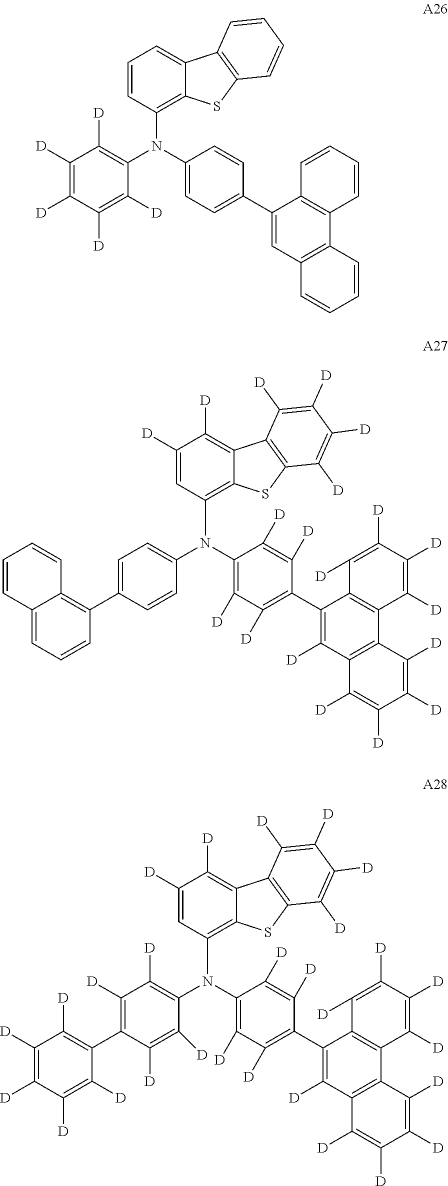 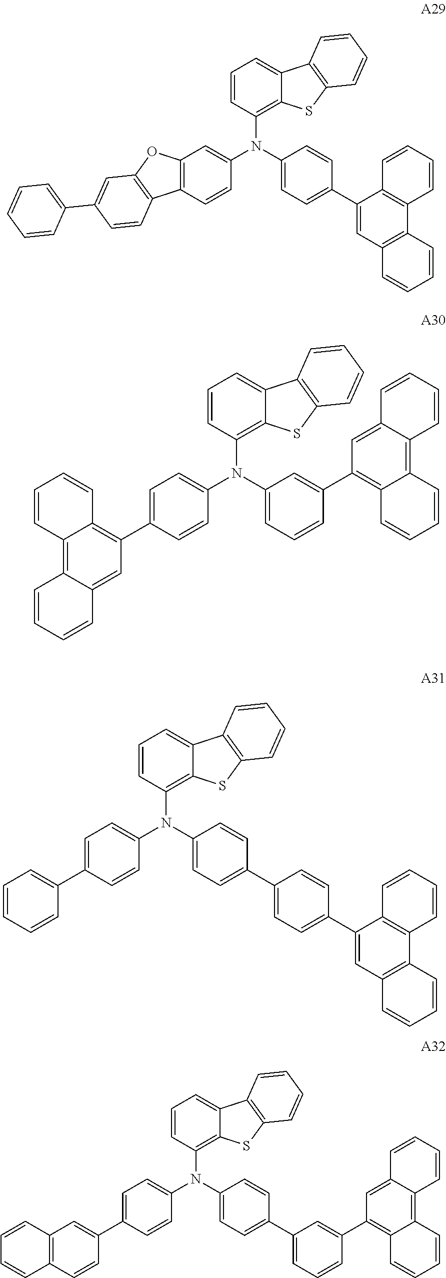 [SMILES: c1ccc(-c2ccc3c(c2)oc2cc(N(c4ccc(-c5cc6ccccc6c6ccccc56)cc4)c4cccc5c4sc4ccccc45)ccc23)cc1 ; c1cc(-c2cc3ccccc3c3ccccc23)cc(N(c2ccc(-c3cc4ccccc4c4ccccc34)cc2)c2cccc3c2sc2ccccc23)c1 ; c1ccc(-c2ccc(N(c3ccc(-c4ccc(-c5cc6ccccc6c6ccccc56)cc4)cc3)c3cccc4c3sc3ccccc34)cc2)cc1 ; c1cc(-c2ccc(N(c3ccc(-c4ccc5ccccc5c4)cc3)c3cccc4c3sc3ccccc34)cc2)cc(-c2cc3ccccc3c3ccccc23)c1] 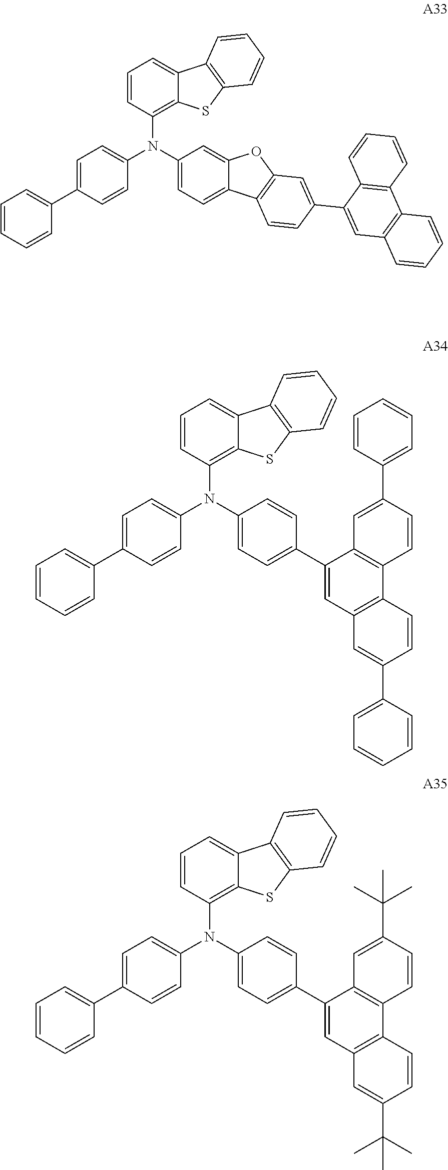 [SMILES: c1ccc(-c2ccc(N(c3ccc4c(c3)oc3cc(-c5cc6ccccc6c6ccccc56)ccc34)c3cccc4c3sc3ccccc34)cc2)cc1 ; c1ccc(-c2ccc(N(c3ccc(-c4cc5cc(-c6ccccc6)ccc5c5ccc(-c6ccccc6)cc45)cc3)c3cccc4c3sc3ccccc34)cc2)cc1 ; CC(C)(C)c1ccc2c(c1)cc(-c1ccc(N(c3ccc(-c4ccccc4)cc3)c3cccc4c3sc3ccccc34)cc1)c1cc(C(C)(C)C)ccc12] 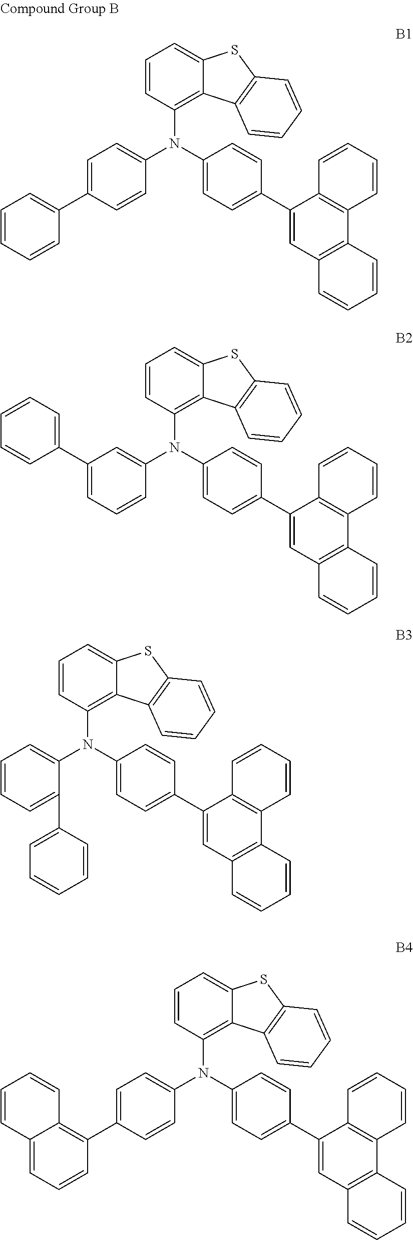 [SMILES: c1ccc(-c2ccc(N(c3ccc(-c4cc5ccccc5c5ccccc45)cc3)c3cccc4sc5ccccc5c34)cc2)cc1 ; c1ccc(-c2cccc(N(c3ccc(-c4cc5ccccc5c5ccccc45)cc3)c3cccc4sc5ccccc5c34)c2)cc1 ; c1ccc(-c2ccccc2N(c2ccc(-c3cc4ccccc4c4ccccc34)cc2)c2cccc3sc4ccccc4c23)cc1 ; c1ccc2c(-c3ccc(N(c4ccc(-c5cc6ccccc6c6ccccc56)cc4)c4cccc5sc6ccccc6c45)cc3)cccc2c1] 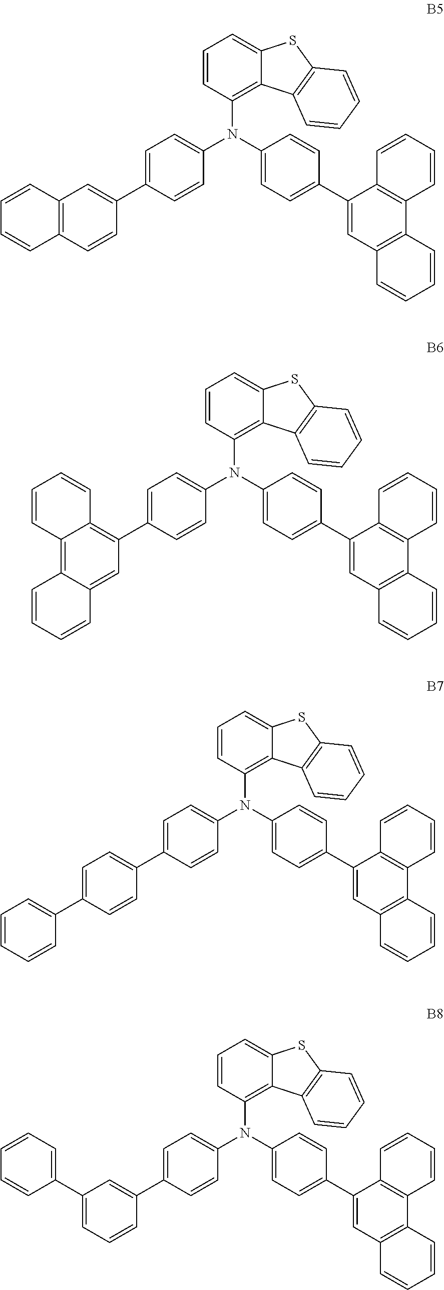 [SMILES: c1ccc2cc(-c3ccc(N(c4ccc(-c5cc6ccccc6c6ccccc56)cc4)c4cccc5sc6ccccc6c45)cc3)ccc2c1 ; c1ccc2c(c1)cc(-c1ccc(N(c3ccc(-c4cc5ccccc5c5ccccc45)cc3)c3cccc4sc5ccccc5c34)cc1)c1ccccc12 ; c1ccc(-c2ccc(-c3ccc(N(c4ccc(-c5cc6ccccc6c6ccccc56)cc4)c4cccc5sc6ccccc6c45)cc3)cc2)cc1 ; c1ccc(-c2cccc(-c3ccc(N(c4ccc(-c5cc6ccccc6c6ccccc56)cc4)c4cccc5sc6ccccc6c45)cc3)c2)cc1] 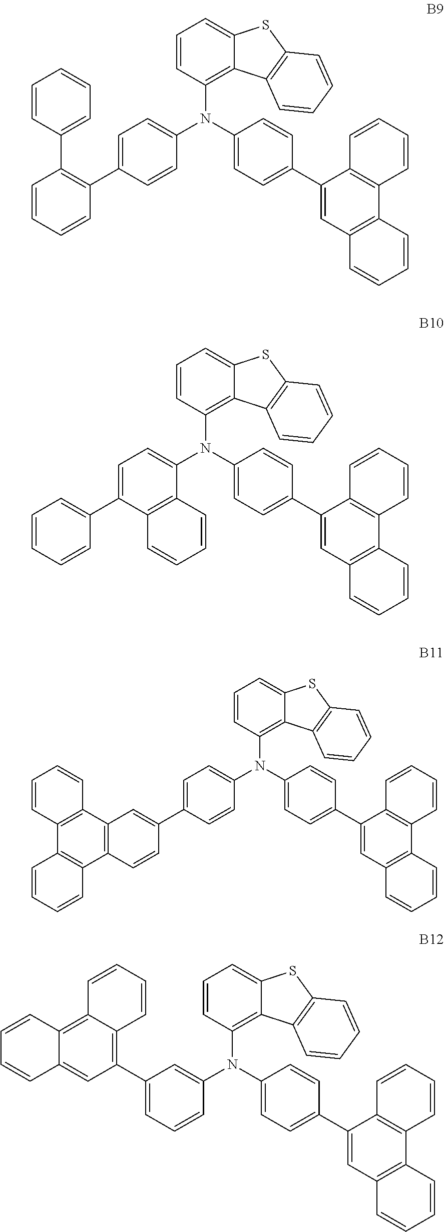 [SMILES: c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)c3ccccc23)cc1 ; c1ccc2c(c1)cc(-c1ccc(N(c3ccc(-c4ccc5c6ccccc6c6ccccc6c5c4)cc3)c3cccc4sc5ccccc5c34)cc1)c1ccccc12 ; c1cc(-c2cc3ccccc3c3ccccc23)cc(N(c2ccc(-c3cc4ccccc4c4ccccc34)cc2)c2cccc3sc4ccccc4c23)c1] 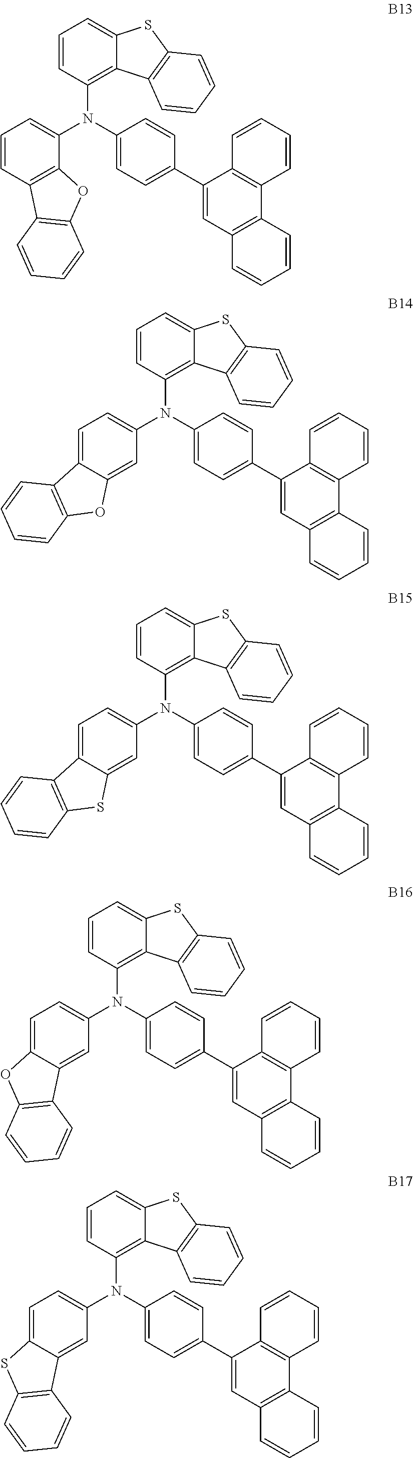 [SMILES: c1ccc2c(c1)cc(-c1ccc(N(c3cccc4c3oc3ccccc34)c3cccc4sc5ccccc5c34)cc1)c1ccccc12 ; c1ccc2c(c1)cc(-c1ccc(N(c3ccc4c(c3)oc3ccccc34)c3cccc4sc5ccccc5c34)cc1)c1ccccc12 ; c1ccc2c(c1)cc(-c1ccc(N(c3ccc4c(c3)sc3ccccc34)c3cccc4sc5ccccc5c34)cc1)c1ccccc12 ; c1ccc2c(c1)cc(-c1ccc(N(c3ccc4oc5ccccc5c4c3)c3cccc4sc5ccccc5c34)cc1)c1ccccc12 ; c1ccc2c(c1)cc(-c1ccc(N(c3ccc4sc5ccccc5c4c3)c3cccc4sc5ccccc5c34)cc1)c1ccccc12] 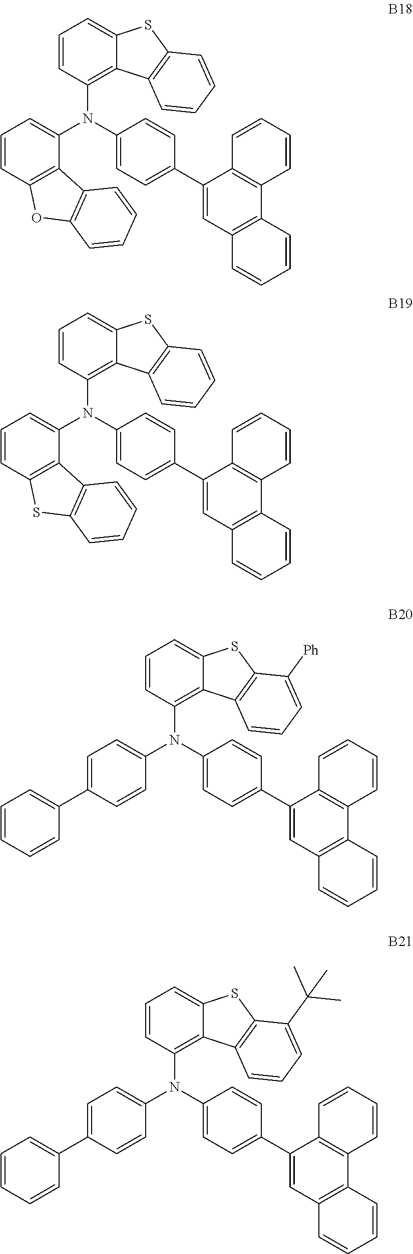 [SMILES: c1ccc2c(c1)cc(-c1ccc(N(c3cccc4oc5ccccc5c34)c3cccc4sc5ccccc5c34)cc1)c1ccccc12 ; c1ccc2c(c1)cc(-c1ccc(N(c3cccc4sc5ccccc5c34)c3cccc4sc5ccccc5c34)cc1)c1ccccc12 ; c1ccc(-c2ccc(N(c3ccc(-c4cc5ccccc5c5ccccc45)cc3)c3cccc4sc5c(-c6ccccc6)cccc5c34)cc2)cc1 ; CC(C)(C)c1cccc2c1sc1cccc(N(c3ccc(-c4ccccc4)cc3)c3ccc(-c4cc5ccccc5c5ccccc45)cc3)c12] 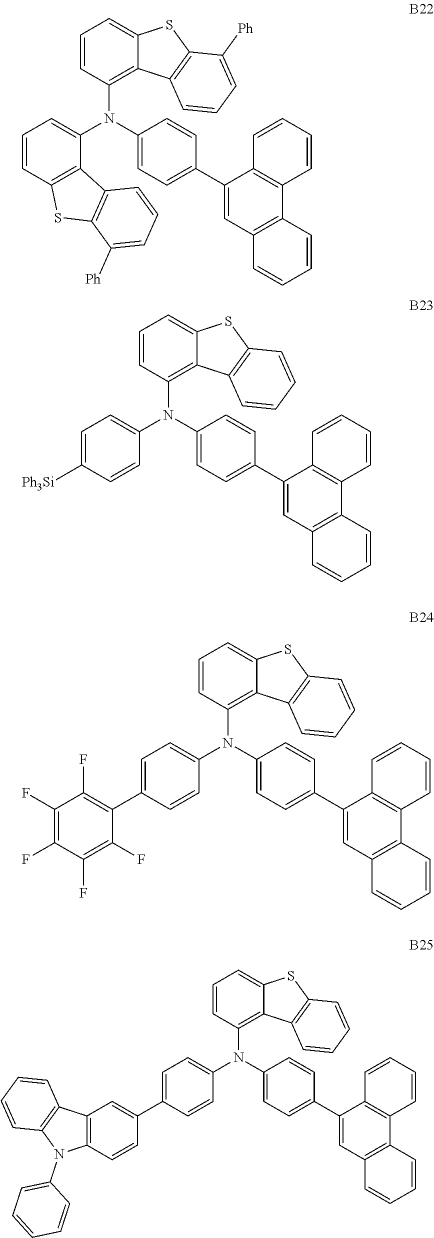 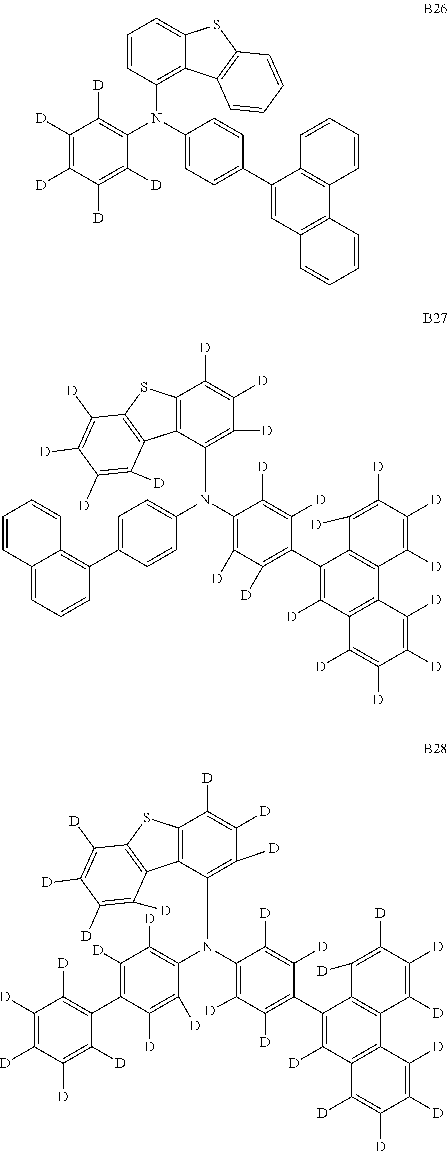 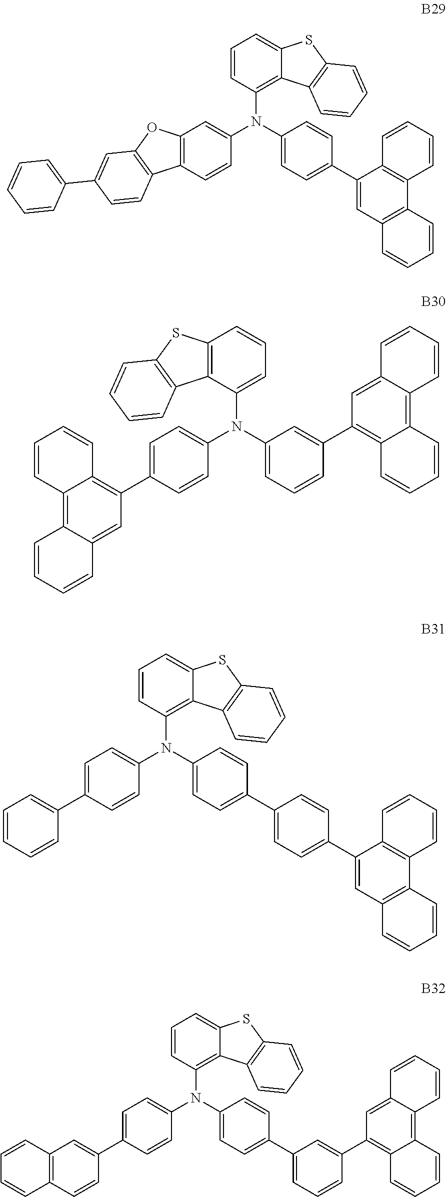 [SMILES: c1ccc(-c2ccc3c(c2)oc2cc(N(c4ccc(-c5cc6ccccc6c6ccccc56)cc4)c4cccc5sc6ccccc6c45)ccc23)cc1 ; c1cc(-c2cc3ccccc3c3ccccc23)cc(N(c2ccc(-c3cc4ccccc4c4ccccc34)cc2)c2cccc3sc4ccccc4c23)c1 ; c1ccc(-c2ccc(N(c3ccc(-c4ccc(-c5cc6ccccc6c6ccccc56)cc4)cc3)c3cccc4sc5ccccc5c34)cc2)cc1 ; c1cc(-c2ccc(N(c3ccc(-c4ccc5ccccc5c4)cc3)c3cccc4sc5ccccc5c34)cc2)cc(-c2cc3ccccc3c3ccccc23)c1] 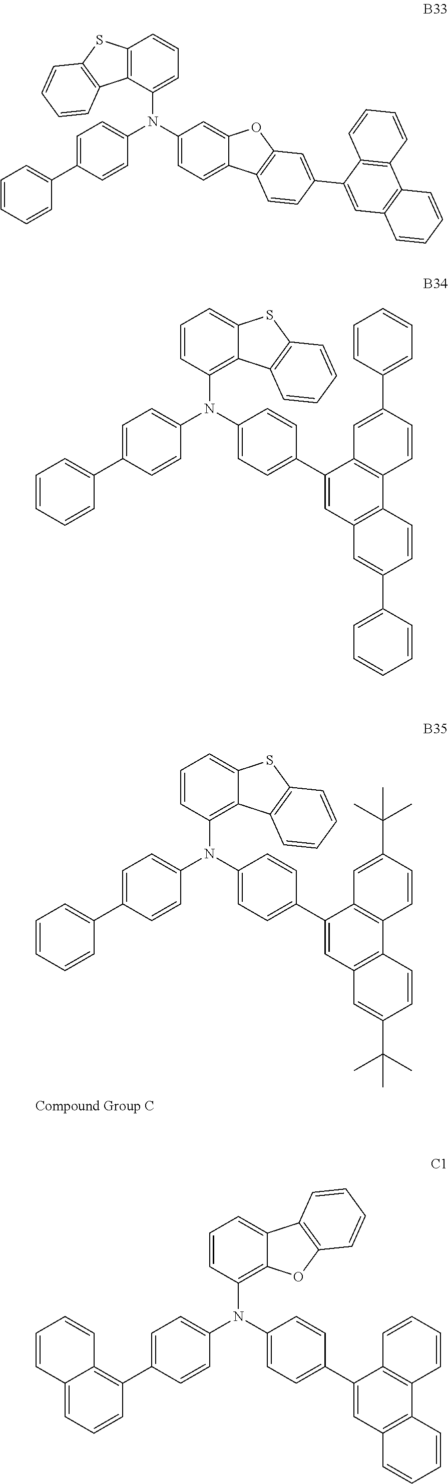 [SMILES: c1ccc(-c2ccc(N(c3ccc4c(c3)oc3cc(-c5cc6ccccc6c6ccccc56)ccc34)c3cccc4sc5ccccc5c34)cc2)cc1 ; c1ccc(-c2ccc(N(c3ccc(-c4cc5cc(-c6ccccc6)ccc5c5ccc(-c6ccccc6)cc45)cc3)c3cccc4sc5ccccc5c34)cc2)cc1 ; CC(C)(C)c1ccc2c(c1)cc(-c1ccc(N(c3ccc(-c4ccccc4)cc3)c3cccc4sc5ccccc5c34)cc1)c1cc(C(C)(C)C)ccc12 ; c1ccc2c(-c3ccc(N(c4ccc(-c5cc6ccccc6c6ccccc56)cc4)c4cccc5c4oc4ccccc45)cc3)cccc2c1] 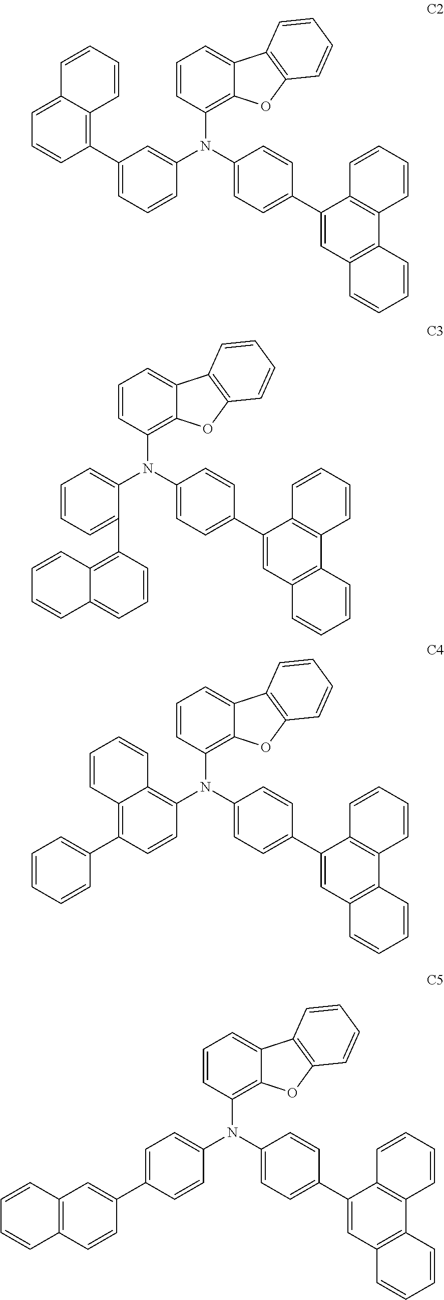 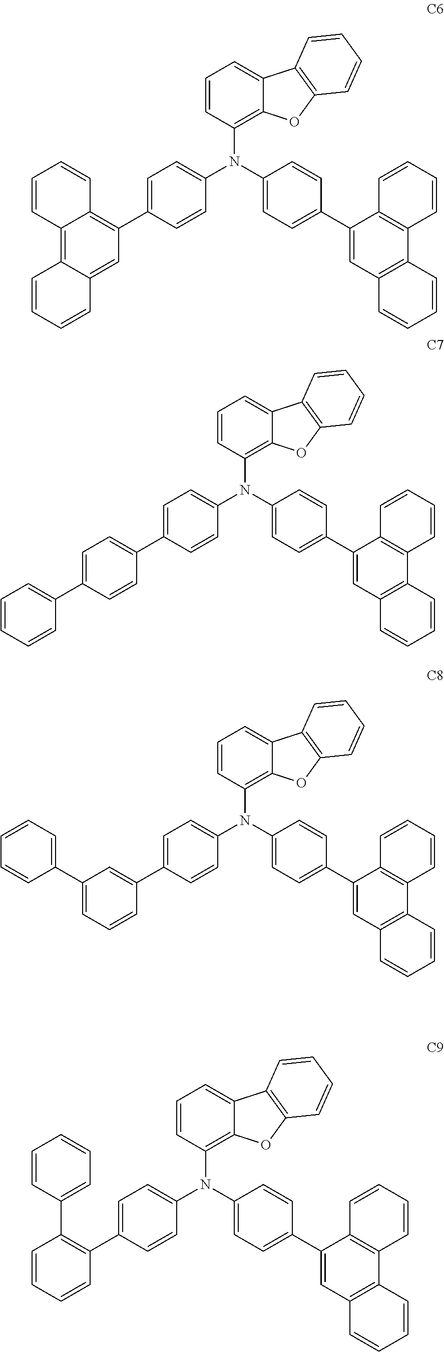 [SMILES: c1ccc2c(c1)cc(-c1ccc(N(c3ccc(-c4cc5ccccc5c5ccccc45)cc3)c3cccc4c3oc3ccccc34)cc1)c1ccccc12 ; c1ccc(-c2ccc(-c3ccc(N(c4ccc(-c5cc6ccccc6c6ccccc56)cc4)c4cccc5c4oc4ccccc45)cc3)cc2)cc1 ; c1ccc(-c2cccc(-c3ccc(N(c4ccc(-c5cc6ccccc6c6ccccc56)cc4)c4cccc5c4oc4ccccc45)cc3)c2)cc1 ; c1ccc(-c2ccccc2-c2ccc(N(c3ccc(-c4cc5ccccc5c5ccccc45)cc3)c3cccc4c3oc3ccccc34)cc2)cc1] 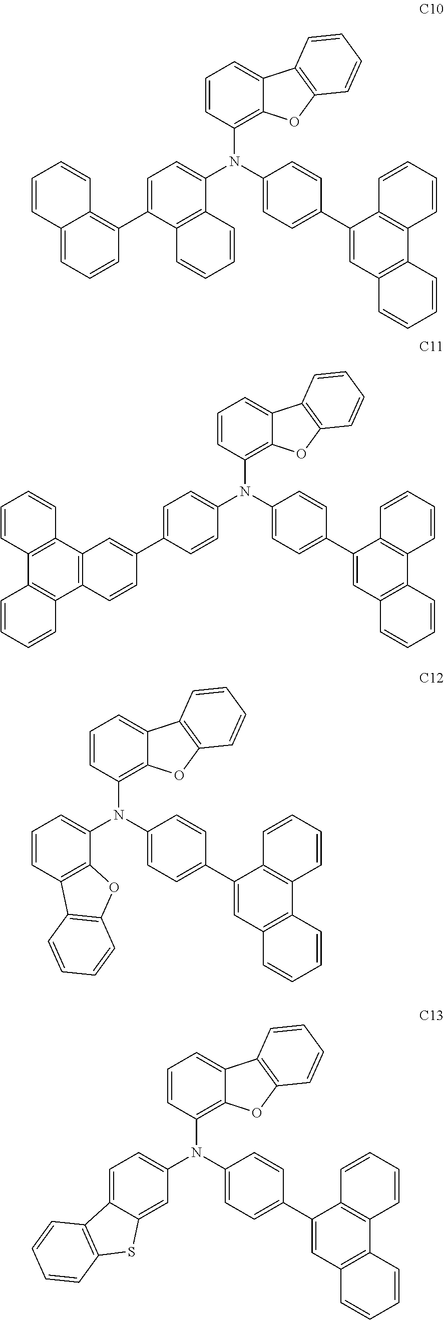 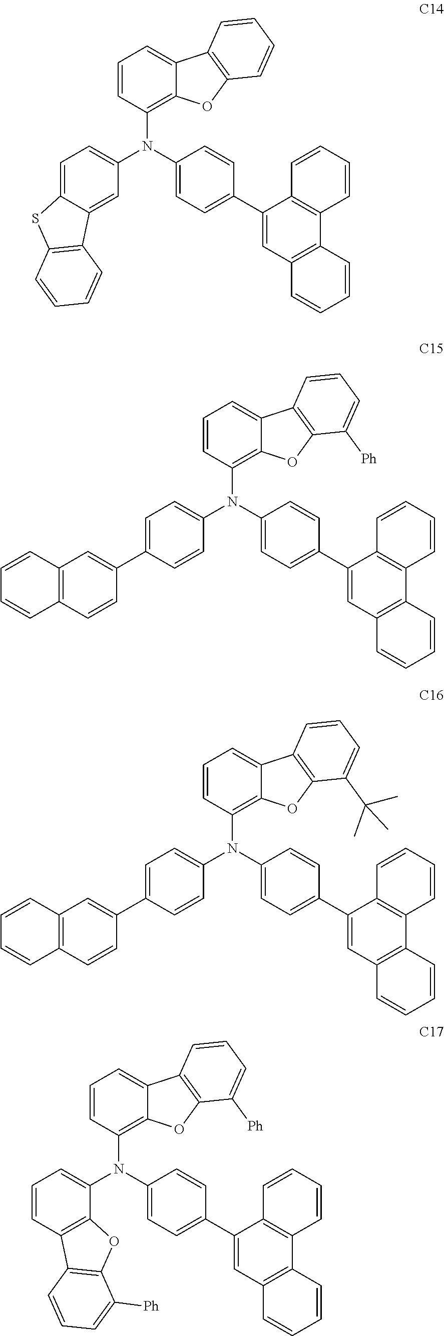 [SMILES: c1ccc2c(c1)cc(-c1ccc(N(c3ccc4sc5ccccc5c4c3)c3cccc4c3oc3ccccc34)cc1)c1ccccc12 ; c1ccc(-c2cccc3c2oc2c(N(c4ccc(-c5ccc6ccccc6c5)cc4)c4ccc(-c5cc6ccccc6c6ccccc56)cc4)cccc23)cc1 ; CC(C)(C)c1cccc2c1oc1c(N(c3ccc(-c4ccc5ccccc5c4)cc3)c3ccc(-c4cc5ccccc5c5ccccc45)cc3)cccc12 ; c1ccc(-c2cccc3c2oc2c(N(c4ccc(-c5cc6ccccc6c6ccccc56)cc4)c4cccc5c4oc4c(-c6ccccc6)cccc45)cccc23)cc1] 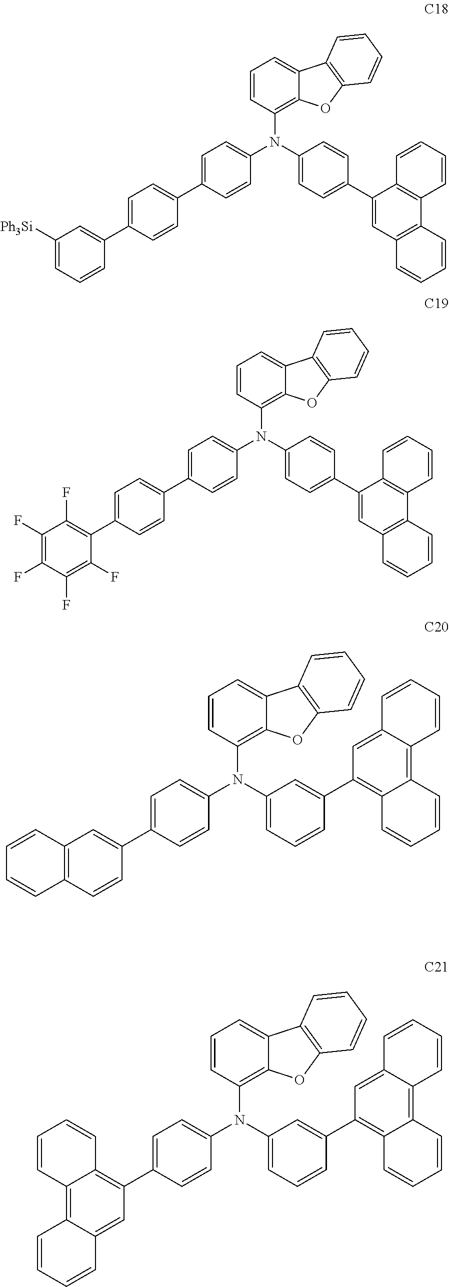 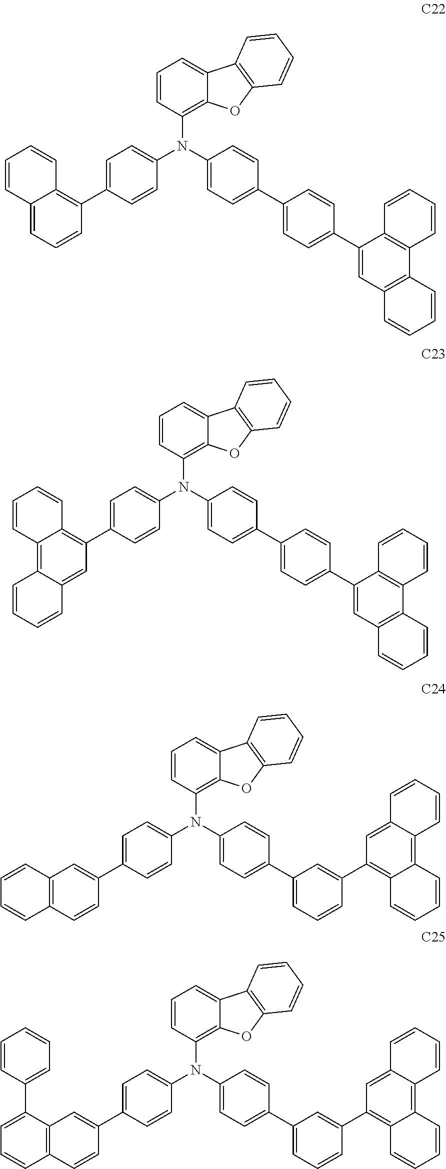 [SMILES: c1ccc2c(-c3ccc(N(c4ccc(-c5ccc(-c6cc7ccccc7c7ccccc67)cc5)cc4)c4cccc5c4oc4ccccc45)cc3)cccc2c1 ; c1ccc2c(c1)cc(-c1ccc(-c3ccc(N(c4ccc(-c5cc6ccccc6c6ccccc56)cc4)c4cccc5c4oc4ccccc45)cc3)cc1)c1ccccc12 ; c1cc(-c2ccc(N(c3ccc(-c4ccc5ccccc5c4)cc3)c3cccc4c3oc3ccccc34)cc2)cc(-c2cc3ccccc3c3ccccc23)c1 ; c1ccc(-c2cccc3ccc(-c4ccc(N(c5ccc(-c6cccc(-c7cc8ccccc8c8ccccc78)c6)cc5)c5cccc6c5oc5ccccc56)cc4)cc23)cc1] 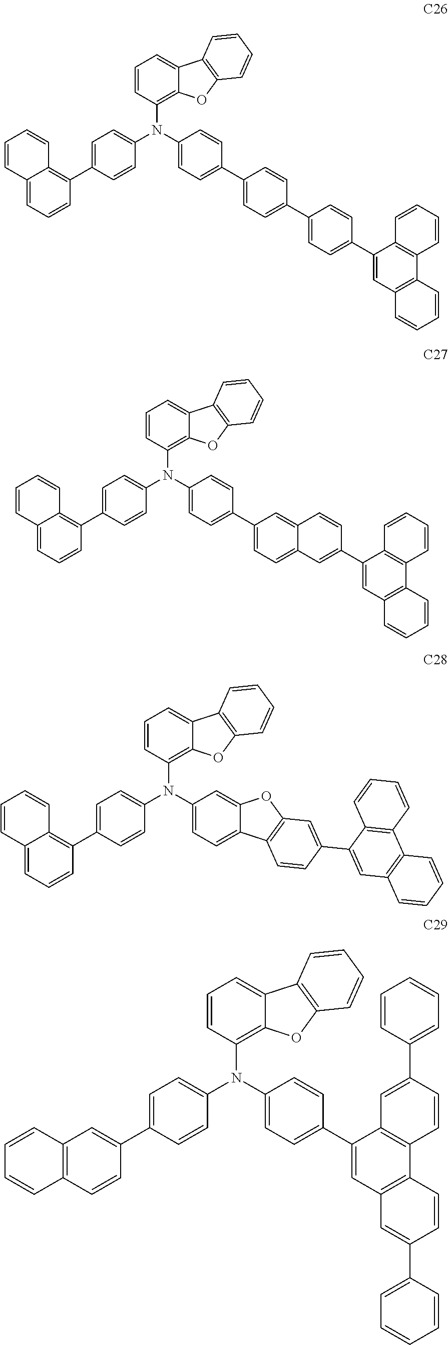 [SMILES: c1ccc2c(-c3ccc(N(c4ccc(-c5ccc(-c6ccc(-c7cc8ccccc8c8ccccc78)cc6)cc5)cc4)c4cccc5c4oc4ccccc45)cc3)cccc2c1 ; c1ccc2c(-c3ccc(N(c4ccc(-c5ccc6cc(-c7cc8ccccc8c8ccccc78)ccc6c5)cc4)c4cccc5c4oc4ccccc45)cc3)cccc2c1 ; c1ccc2c(-c3ccc(N(c4ccc5c(c4)oc4cc(-c6cc7ccccc7c7ccccc67)ccc45)c4cccc5c4oc4ccccc45)cc3)cccc2c1 ; c1ccc(-c2ccc3c(c2)cc(-c2ccc(N(c4ccc(-c5ccc6ccccc6c5)cc4)c4cccc5c4oc4ccccc45)cc2)c2cc(-c4ccccc4)ccc23)cc1] 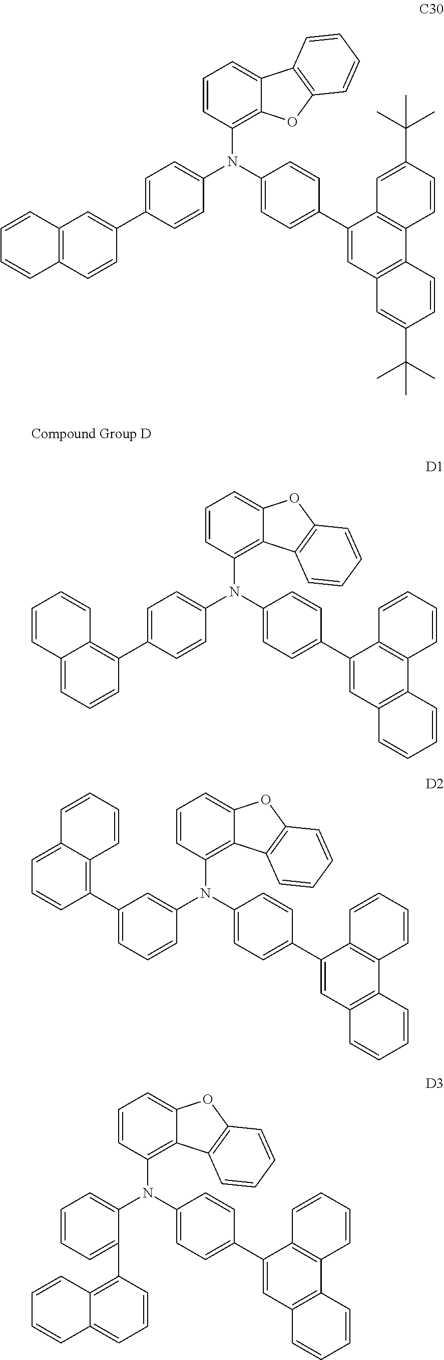 [SMILES: CC(C)(C)c1ccc2c(c1)cc(-c1ccc(N(c3ccc(-c4ccc5ccccc5c4)cc3)c3cccc4c3oc3ccccc34)cc1)c1cc(C(C)(C)C)ccc12 ; c1ccc2c(-c3ccc(N(c4ccc(-c5cc6ccccc6c6ccccc56)cc4)c4cccc5oc6ccccc6c45)cc3)cccc2c1 ; c1cc(-c2cccc3ccccc23)cc(N(c2ccc(-c3cc4ccccc4c4ccccc34)cc2)c2cccc3oc4ccccc4c23)c1 ; c1ccc(N(c2ccc(-c3cc4ccccc4c4ccccc34)cc2)c2cccc3oc4ccccc4c23)c(-c2cccc3ccccc23)c1] 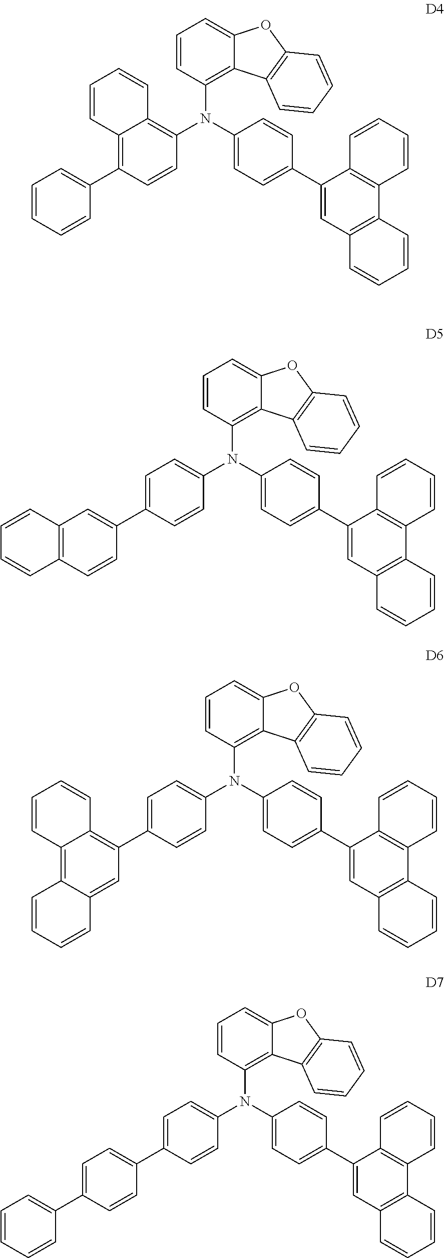 [SMILES: c1ccc(-c2ccc(N(c3ccc(-c4cc5ccccc5c5ccccc45)cc3)c3cccc4oc5ccccc5c34)c3ccccc23)cc1 ; c1ccc2cc(-c3ccc(N(c4ccc(-c5cc6ccccc6c6ccccc56)cc4)c4cccc5oc6ccccc6c45)cc3)ccc2c1 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc(-c2ccc(-c3ccc(N(c4ccc(-c5cc6ccccc6c6ccccc56)cc4)c4cccc5oc6ccccc6c45)cc3)cc2)cc1] 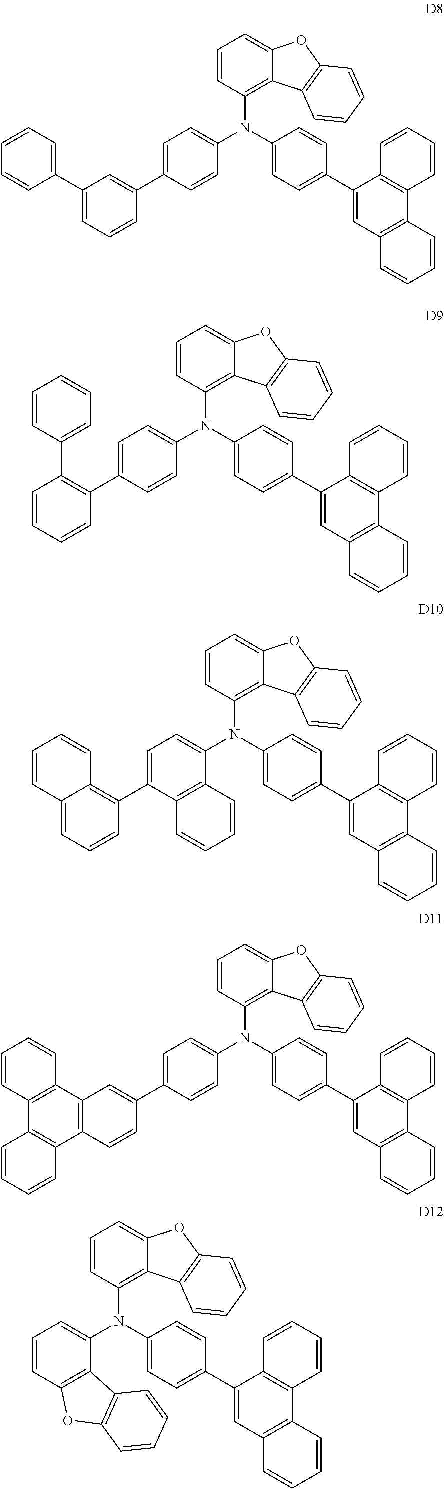 [SMILES: c1ccc(-c2cccc(-c3ccc(N(c4ccc(-c5cc6ccccc6c6ccccc56)cc4)c4cccc5oc6ccccc6c45)cc3)c2)cc1 ; c1ccc(-c2ccccc2-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)c4ccccc34)cccc2c1 ; c1ccc2c(c1)cc(-c1ccc(N(c3ccc(-c4ccc5c6ccccc6c6ccccc6c5c4)cc3)c3cccc4oc5ccccc5c34)cc1)c1ccccc12 ; c1ccc2c(c1)cc(-c1ccc(N(c3cccc4oc5ccccc5c34)c3cccc4oc5ccccc5c34)cc1)c1ccccc12] 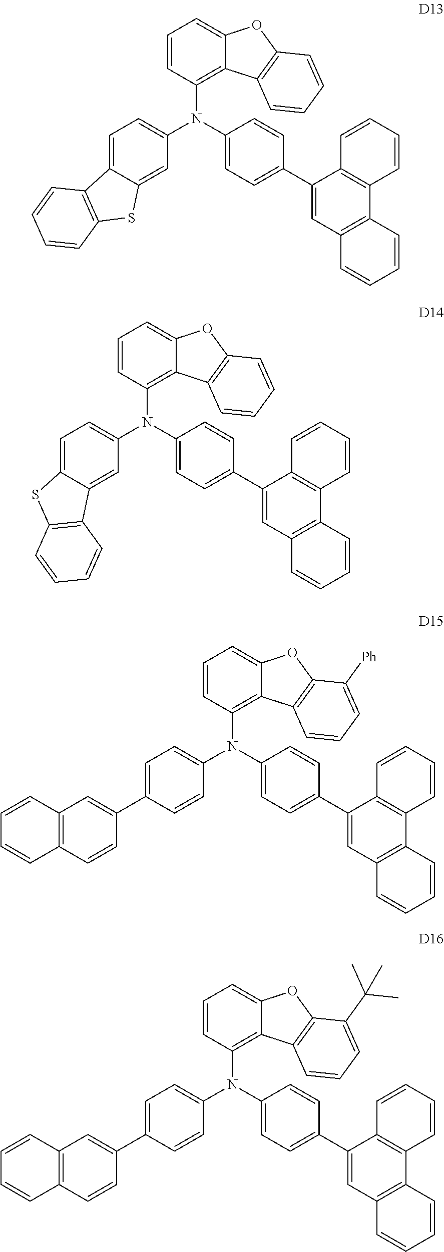 [SMILES: c1ccc2c(c1)cc(-c1ccc(N(c3ccc4c(c3)sc3ccccc34)c3cccc4oc5ccccc5c34)cc1)c1ccccc12 ; c1ccc2c(c1)cc(-c1ccc(N(c3ccc4sc5ccccc5c4c3)c3cccc4oc5ccccc5c34)cc1)c1ccccc12 ; c1ccc(-c2cccc3c2oc2cccc(N(c4ccc(-c5ccc6ccccc6c5)cc4)c4ccc(-c5cc6ccccc6c6ccccc56)cc4)c23)cc1 ; CC(C)(C)c1cccc2c1oc1cccc(N(c3ccc(-c4ccc5ccccc5c4)cc3)c3ccc(-c4cc5ccccc5c5ccccc45)cc3)c12] 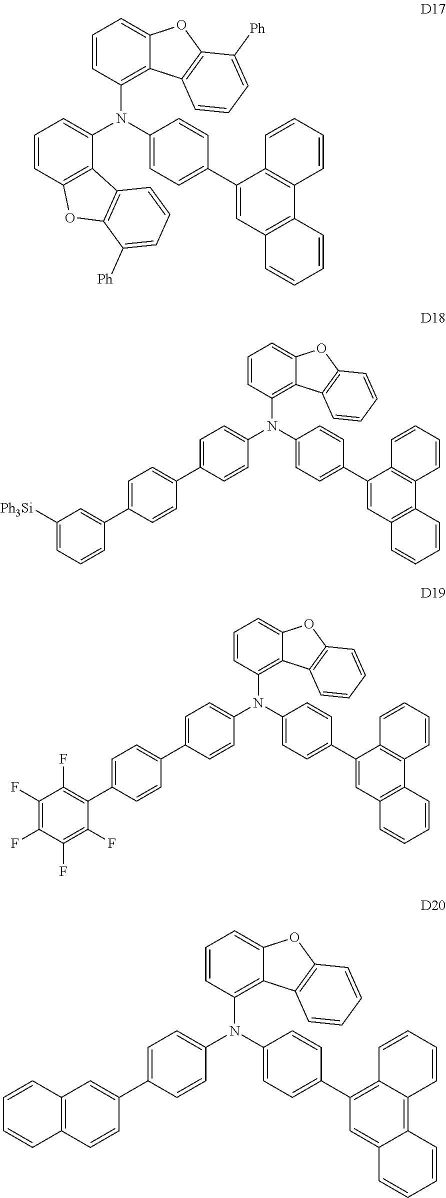 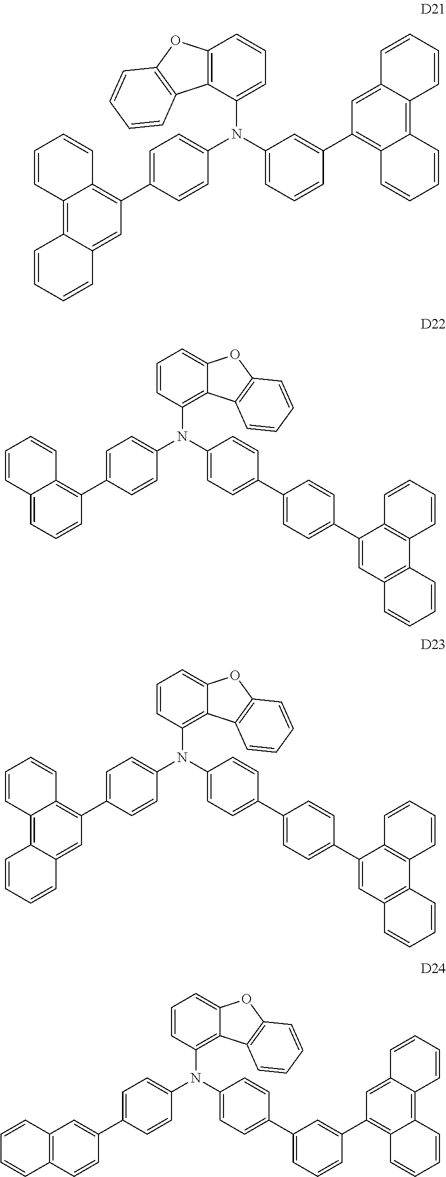 [SMILES: c1cc(-c2cc3ccccc3c3ccccc23)cc(N(c2ccc(-c3cc4ccccc4c4ccccc34)cc2)c2cccc3oc4ccccc4c23)c1 ; c1ccc2c(-c3ccc(N(c4ccc(-c5ccc(-c6cc7ccccc7c7ccccc67)cc5)cc4)c4cccc5oc6ccccc6c45)cc3)cccc2c1 ; c1ccc2c(c1)cc(-c1ccc(-c3ccc(N(c4ccc(-c5cc6ccccc6c6ccccc56)cc4)c4cccc5oc6ccccc6c45)cc3)cc1)c1ccccc12 ; c1cc(-c2ccc(N(c3ccc(-c4ccc5ccccc5c4)cc3)c3cccc4oc5ccccc5c34)cc2)cc(-c2cc3ccccc3c3ccccc23)c1] 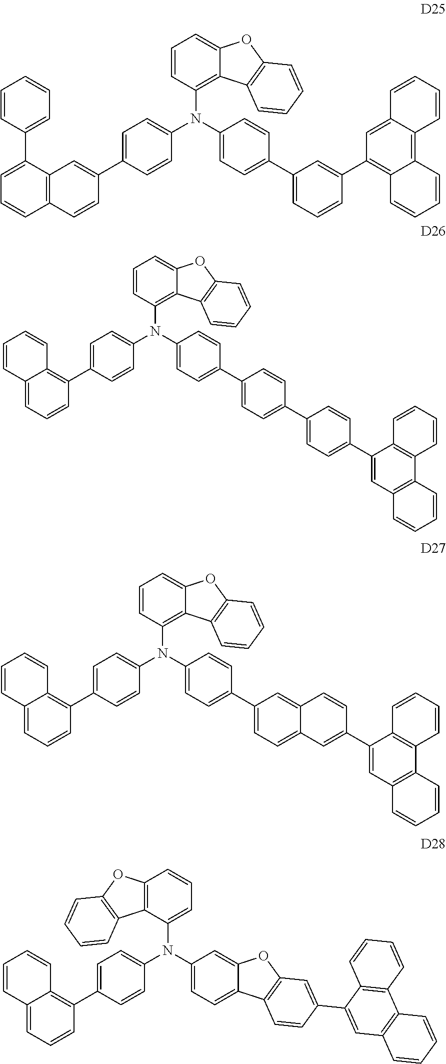 [SMILES: c1ccc(-c2cccc3ccc(-c4ccc(N(c5ccc(-c6cccc(-c7cc8ccccc8c8ccccc78)c6)cc5)c5cccc6oc7ccccc7c56)cc4)cc23)cc1 ; c1ccc2c(-c3ccc(N(c4ccc(-c5ccc(-c6ccc(-c7cc8ccccc8c8ccccc78)cc6)cc5)cc4)c4cccc5oc6ccccc6c45)cc3)cccc2c1 ; c1ccc2c(-c3ccc(N(c4ccc(-c5ccc6cc(-c7cc8ccccc8c8ccccc78)ccc6c5)cc4)c4cccc5oc6ccccc6c45)cc3)cccc2c1 ; c1ccc2c(-c3ccc(N(c4ccc5c(c4)oc4cc(-c6cc7ccccc7c7ccccc67)ccc45)c4cccc5oc6ccccc6c45)cc3)cccc2c1] 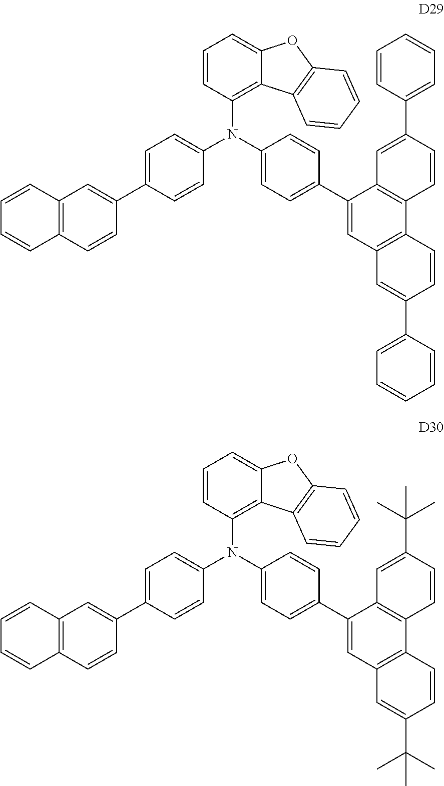 [SMILES: c1ccc(-c2ccc3c(c2)cc(-c2ccc(N(c4ccc(-c5ccc6ccccc6c5)cc4)c4cccc5oc6ccccc6c45)cc2)c2cc(-c4ccccc4)ccc23)cc1 ; CC(C)(C)c1ccc2c(c1)cc(-c1ccc(N(c3ccc(-c4ccc5ccccc5c4)cc3)c3cccc4oc5ccccc5c34)cc1)c1cc(C(C)(C)C)ccc12]
